(19) United States
(12) Patent Application Publication
(10) Pub. No.: US 2026/0053934 A1
(43) Pub. Date: Feb. 26, 2026

(54) DUAL ACTION ONLY PROTEOLYSIS TARGETING CHIMERAS AND USES THEREOF

(71) Applicant: UNIVERSITY OF MASSACHUSETTS, Westborough, MA (US)

(72) Inventors: Sankaran Thayumanavan, Westborough, MA (US); Ranit Dutta, Westborough, MA (US)

(21) Appl. No.: 19/237,137

(22) Filed: Jun. 13, 2025

Related U.S. Application Data
(60) Provisional application No. 63/684,976, filed on Aug. 20, 2024.

(51) Int. Cl. A61K 47/55; A61P 35/00

(52) U.S. Cl. CPC A61K 47/55; A61P 35/00

(57) ABSTRACT

A proteolysis targeting chimera is provided of Formula I:

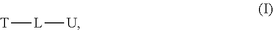

wherein: T includes a target binding moiety capable of binding to a target protein; L includes a linker moiety; and U includes a ubiquitin ligase-recruiting moiety capable of binding a ubiquitin ligase; wherein any of T, L, or U further includes a first stimulus-reactive moiety S1, wherein S1 is reactive to a first stimulus; and wherein any of T, L, or U further includes a second stimulus-reactive moiety S2, wherein S2 is reactive to a second stimulus, wherein the second stimulus is different than the first stimulus.

### STATEMENT REGARDING FEDERALLY SPONSORED RESEARCH OR DEVELOPMENT

This application was made with government support under Grant No. GM-136395 awarded by the National Institutes of Health. The Government has certain rights in the invention.

### BACKGROUND

Targeted protein degraders like PROTACs (Proteolysis Targeting Chimeras) are gaining attention for their ability to degrade specific proteins, but off-target effects pose challenges. Improving selectivity, especially against tumors, is crucial. Prodrug-based strategies show promise, but single-stimulus pro-PROTACs can still cause unintended toxicity.

Therefore, balancing specificity while avoiding off-target effects remains a key challenge in developing effective targeted protein degradation therapies. This disclosure addresses this as well as other needs.

### SUMMARY

The present disclosure provides compounds, compositions, and methods of making and using the disclosed compounds and compositions. More particularly, the present disclosure provides proteolysis targeting chimeras (PROTACs), pharmaceutical compositions comprising said PROTACs, and methods of treating medical disorders with said PROTACs.

In one aspect, a proteolysis targeting chimera (PROTAC) is provided. In some aspects, the PROTAC is of Formula I:

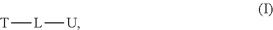

or a pharmaceutically acceptable salt or derivative thereof.

T as found in Formula I can include a target binding moiety. The target binding moiety can be capable of binding to a target protein. L as found in Formula I can include a linker moiety. U as found in Formula I can include a ubiquitin ligase-recruiting moiety. The ubiquitin ligase-recruiting moiety can be capable of binding a ubiquitin ligase.

Any of T, L, or U as found in Formula I can further include a first stimulus-reactive moiety S1. S1 can be reactive to a first stimulus. Any of T, L, or U as found in Formula I can further include a second stimulus-reactive moiety S2. S2 can be reactive to a second stimulus. The second stimulus can be different than the first stimulus.

Upon exposure to only the first stimulus, only the second stimulus, or to neither of the first stimulus or second stimulus, the PROTAC of Formula I does not substantially recruit the target protein for ubiquitination and degradation by a proteasome.

Upon exposure to both the first stimulus and the second stimulus, the PROTAC of Formula I forms an active compound capable of recruiting the target protein for ubiquitination and degradation by a proteasome.

In another aspect, a pharmaceutical composition is provided. The pharmaceutical composition can include a PROTAC as described herein, or a pharmaceutically acceptable salt or derivative thereof, and a pharmaceutically acceptable carrier or excipient.

In another aspect, a method of treating a medical disorder in a subject in need thereof is provided. The medical disorder can be mediated by a target protein. The method can include administering a therapeutically effective amount of a PROTAC as described herein, or a pharmaceutically acceptable salt or derivative thereof, or a pharmaceutical composition as described herein. The target protein is degraded or downregulated by the PROTAC upon administration to the subject.

The details of one or more aspects of the disclosure are set forth in the accompanying drawings and the description below. Other features, objects, and advantages of the disclosure will be apparent from the description, the drawings, and the claims.

### DESCRIPTION OF DRAWINGS

FIG. 1 provides a schematic illustration of a representative dual triggered, viz. hypoxia and Cath-L-responsive activation of DAO-PROTAC to generate a functional PROTAC, prompting proteasomal degradation of the POI. Presence of single stimulus does not result in POI degradation indicating the requirement of an AND-gated stimuli.

FIG. 2 depicts a scheme regarding the mechanisms of hypoxia sensitive reduction and self-immolative cleavage of 4-nitrobenzyl functionality and Cath-L triggered unmasking of ε-acetyl-N-Boc-lysine functionality in DAO-dBET1 as described in the examples. Prior to Cath-L based cleavage of N-Boc-lysine, histone deacetylases (HDACs) intracellularly deacetylate ε-acetyl moiety to release Cath-L responsive substrate.

FIGS. 3A and 3B provide data regarding BRD4 protein degradation using DAO-dBET1 in (FIG. 3A) presence and (FIG. 3B) absence of hypoxia and Cath-L activity in MDA-MB-231 cells as described in the examples. FIG. 3C provides data regarding % Cell viability vs log [Concentration (nM)] of dBET1 and DAO-dBET1 in MDA-MB-231 cells in the presence and absence of hypoxia and Cath-L activity as described in the examples. FIG. 3D provides data regarding a cell apoptosis assay in MDA-MB-231 cells through annexin V and PI staining using flow cytometry post 72 h treatment of (I) DMSO [Cath-L(+); Hypoxia(+)], (II) dBET1 [Cath-L(+); Hypoxia(+)], (III) DAO-dBET1 [Cath-L(+); Hypoxia(+)], (IV) DMSO [Cath-L(−); Hypoxia(−)], (V) DAO-dBET1 [Cath-L(−); Hypoxia(−)] as described in the examples. Green and grey sections collectively indicate total apoptosis, a measure of cytotoxicity.

FIG. 4A provides data regarding BRD4 protein degradation using DAO-dBET1 in normoxic/Cath-L active and hypoxic/Cath-L inhibited conditions in MDA-MB-231 cells as described in the examples. FIG. 4B provides data regarding % Cell viability vs log [Concentration (nM)] of DAO-dBET1 in absence of hypoxia and Cath-L activity, and in the presence of either hypoxia or Cath-L in MDA-MB-231 cells as described in the examples. FIGS. 4C and 4D provide data regarding a cell apoptosis assay through (FIG. 4C) annexin V and PI staining using flow cytometry and (FIG. 4D) Caspase-Glo 3/7 activity post 72 h treatment of (I) DMSO [Cath-L(−); Hypoxia(+)], (II) DAO-dBET1 [Cath-L(−); Hypoxia(+)], (III) DMSO [Cath-L(+); Hypoxia(−)], (IV) DAO-dBET1 [Cath-L(+); Hypoxia(−)] in MDA-MB-231 cells as described in the examples. Green and grey sections collectively indicate total apoptosis, a measure of cytotoxicity. Statistical significance calculated by two-way ANOVA. *p<0.1, **p<0.01, ***p<0.001, ****p<0.0001.

FIG. 5A provides a schematic illustration of Hyp-dBET1 (left) and Cath-L-dBET1 (right). FIG. 5B provides data regarding BRD4 protein degradation using Hyp-dBET1 in normoxic/Cath-L active and Cath-L dBET1 in hypoxic/Cath-L inhibited conditions in MDA-MB-231 cells as described in the examples. FIG. 5C provides data regarding the change in ZFP-91 protein expression using Hyp-dBET1 in normoxic/Cath-L active and thalidomide-O—COOH (TOC) and c-Myc protein expression using Cath-L dBET1 in hypoxic/Cath-L inhibited condition and JQ1 in MDA-MB-231 cells as described in the examples. FIG. 5D provides data regarding the change in ZFP-91 protein expression and c-Myc protein expression using DAO-dBET1 in normoxic/Cath-L inhibited conditions in MDA-MB-231 cells as described in the examples.

FIG. 6A provides data regarding BRD4 protein degradation using DAO-dBET1 in hypoxic/Cath-L active conditions in HeLa cells as described in the examples. FIG. 6B provides data regarding BRD4 degradation using DAO-dBET1 in normoxic/Cath-L inhibited conditions in HeLa cells. FIG. 6C provides data regarding cell viability vs log [Concentration (nM)] of dBET1 and DAO-dBET1 in absence of hypoxia and Cath-L activity as described in the examples. FIG. 6D provides data regarding a cell apoptosis assay through annexin V and PI staining using flow cytometry post 72 h treatment of (I) DMSO [Cath-L(+); Hypoxia(+)], (II) dBET1 [Cath-L(+); Hypoxia(+)], (III) DAO-dBET1 [Cath-L(+); Hypoxia(+)], (IV) DMSO [Cath-L(−); Hypoxia(−)], (V) DAO-dBET1 [Cath-L(−); Hypoxia(−)] as described in the examples. FIG. 6E provides data regarding BRD4 protein degradation using DAO-dBET1 in normoxic/Cath-L active and hypoxic/Cath-L inhibited conditions in HeLa cells as described in the examples. FIG. 6F provides data regarding % Cell viability as a function of log [Concentration (nM)] of dBET1 and DAO-dBET1 in absence of hypoxia and Cath-L activity as described in the examples. FIG. 6G provides data regarding cell apoptosis assays in HeLa cells through annexin V and PI staining through flow cytometry post 72 h treatment of (I) DMSO [Cath-L(−); Hypoxia(+)], (II) DAO-dBET1 [Cath-L(−); Hypoxia(+)], (III) DMSO [Cath-L(+); Hypoxia(−)], (IV) DAO-dBET1 [Cath-L(+); Hypoxia(−)] as described in the examples. Green and grey sections collectively indicate total apoptosis, a measure of cytotoxicity.

FIGS. 7A and 7B provide data regarding BRD4 protein degradation using DAO-dBET1 in hypoxic and normoxic conditions in HEK-293 cells as described in the examples. FIG. 7C provides data regarding % Cell viability in HEK-293 cells vs log [Concentration (nM)] of dBET1 and DAO-dBET1 in presence and absence of hypoxia in HEK-293 cells as described in the examples. FIG. 7D provides data regarding cell apoptosis assays through caspase 3/7 activity post 72 h treatment of (I) DMSO [Hypoxia(+)], (II) dBET1 [Hypoxia(+)], (III) DAO-dBET1 [Hypoxia(+)], (IV) DMSO [Hypoxia(−)], (V) DAO-dBET1 [Hypoxia(−)] in HEK-293 cells as described in the examples. Statistical significance calculated by two-way ANOVA. *p<0.1, **p<0.01, ***p<0.001, ****p<0.0001. FIG. 7E provides data regarding the change in c-Myc protein expression using DAO-dBET1 in hypoxic (left) and normoxic (right) conditions in HEK-293 cells as described in the examples. FIG. 7F provides data regarding the change in ZFP-91 protein level using DAO-dBET1 under normoxia in HEK-293 cells as described in the examples.

FIG. 8 depicts and provides data regarding a Western blot analysis of BRD4 degradation in MDA-MB-231 cells by treating dBET1 for 24 h as described in the examples.

FIG. 9 depicts and provides data regarding a Western blot analysis of BRD4 degradation by treating DAO-dBET1 for 24 h in MDA-MB-231 cells after inducing hypoxic condition using 200 μM CoCl₂ solution (Cath-L active) as described in the examples.

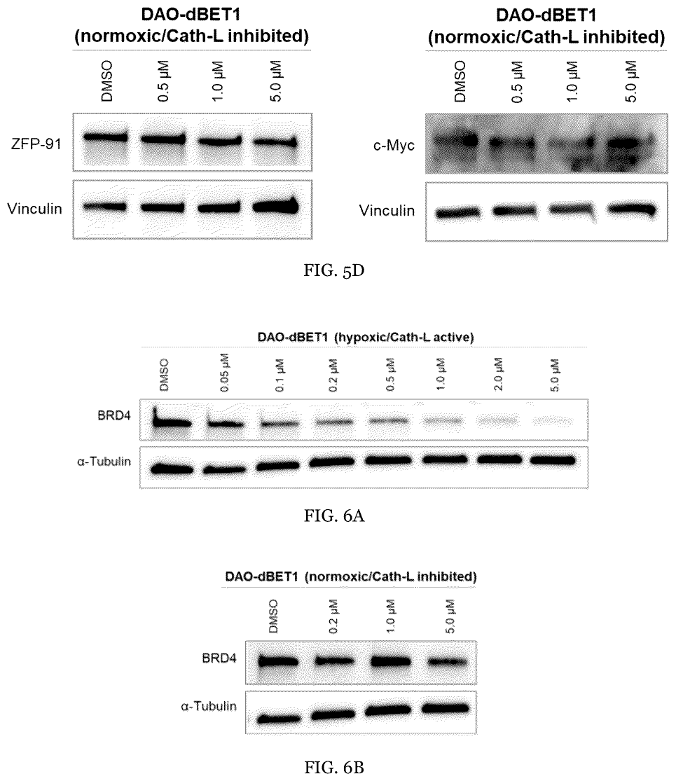

FIG. 10 depicts and provides data regarding a Western blot analysis of BRD4 degradation by treating DAO-dBET1 for 24 h in MDA-MB-231 cells for the following conditions: (i) in presence of either hypoxia or Cath-L activity (left) and (ii) in absence of both the triggers (right) as described in the examples.

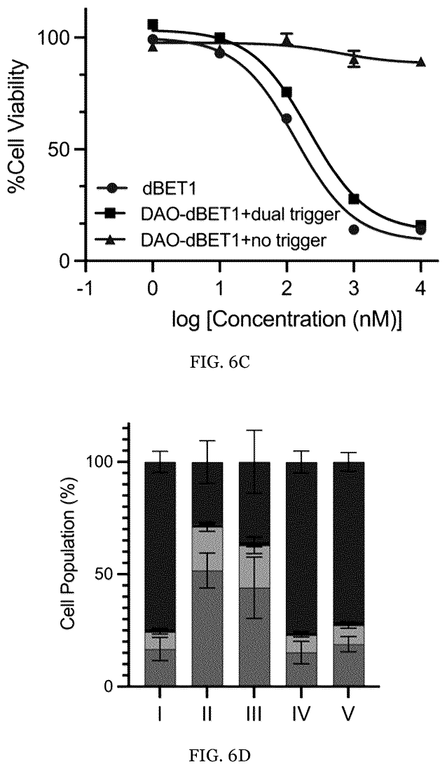

FIGS. 11A to 11C depict quantification of Western blots described above. FIG. 11A provides data regarding the percent BRD4 degradation by treating DAO-dBET1 for 24 h in MDA-MB-231 cells after inducing hypoxic condition using 200 μM CoCl₂ solution (Cath-L active) and comparison with BRD4 degradation by dBET1 treatment in the same conditions as described in the examples. FIG. 11B provides data regarding the percent BRD4 degradation by treating DAO-dBET1 for 24 h in MDA-MB-231 cells in presence of either hypoxia or Cath-L activity as described in the examples. FIG. 11C provides data regarding the percent BRD4 degradation by treating DAO-dBET1 for 24 h in MDA-MB-231 cells in absence of both the triggers as described in the examples.

FIG. 12 depicts and provides data regarding a Western blot analysis of BRD4 degradation in HeLa cells by treating dBET1 for 24 h as described in the examples.

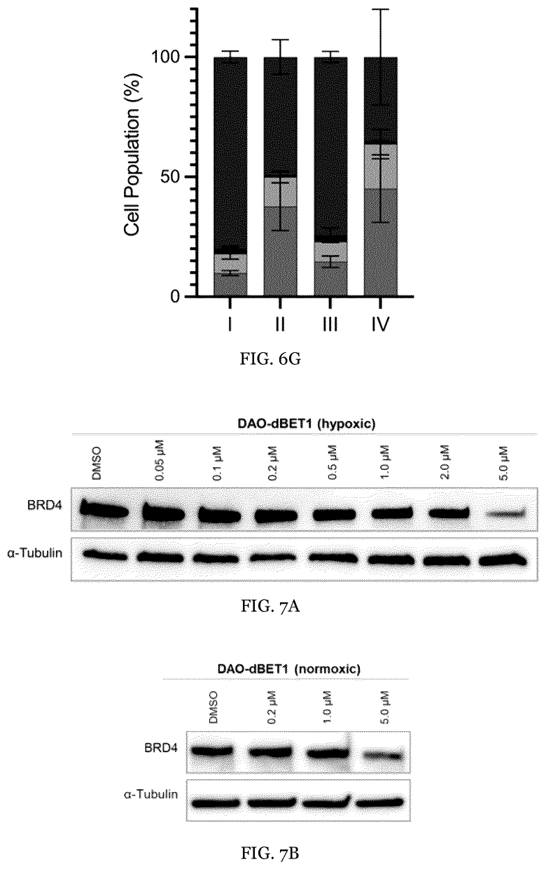

FIG. 13 depicts and provides data regarding a Western blot analysis of BRD4 degradation by treating DAO-dBET1 for 24 h in HeLa cells after inducing hypoxic condition using 200 μM CoCl₂ solution (Cath-L active) as described in the examples.

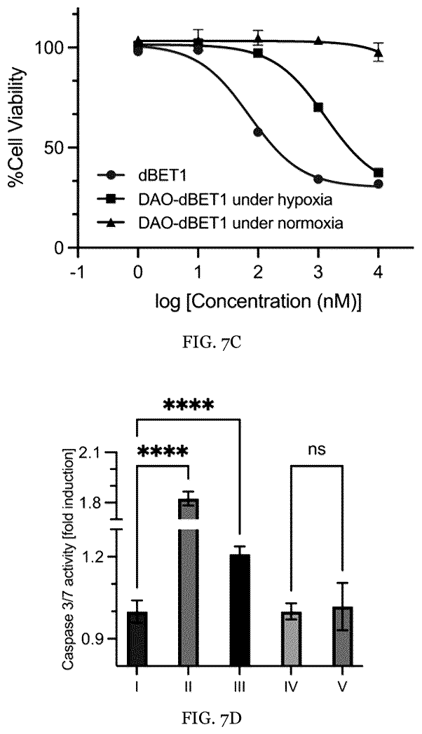

FIG. 14 depicts and provides data regarding a Western blot analysis of BRD4 degradation by treating DAO-dBET1 for 24 h in HeLa cells for the following conditions: (i) in presence of either hypoxia or Cath-L activity (left) and (ii) in absence of both the triggers (right) as described in the examples.

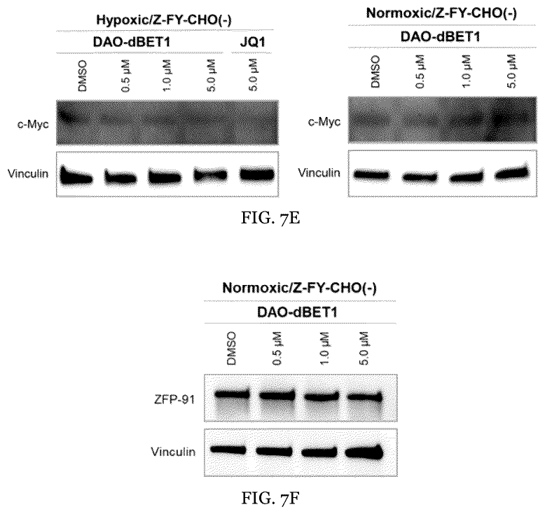

FIGS. 15A to 15C depict quantification of Western blots described above. FIG. 15A provides data regarding the percent BRD4 degradation by treating DAO-dBET1 for 24 h in HeLa cells after inducing hypoxic condition using 200 μM CoCl₂ solution (Cath-L active) and comparison with BRD4 degradation by dBET1 treatment in the same conditions as described in the examples. FIG. 15B provides data regarding the percent BRD4 degradation by treating DAO-dBET1 for 24 h in HeLa cells in presence of either hypoxia or Cath-L activity as described in the examples. FIG. 15C provides data regarding the percent BRD4 degradation by treating DAO-dBET1 for 24 h in HeLa cells in absence of both the triggers as described in the examples.

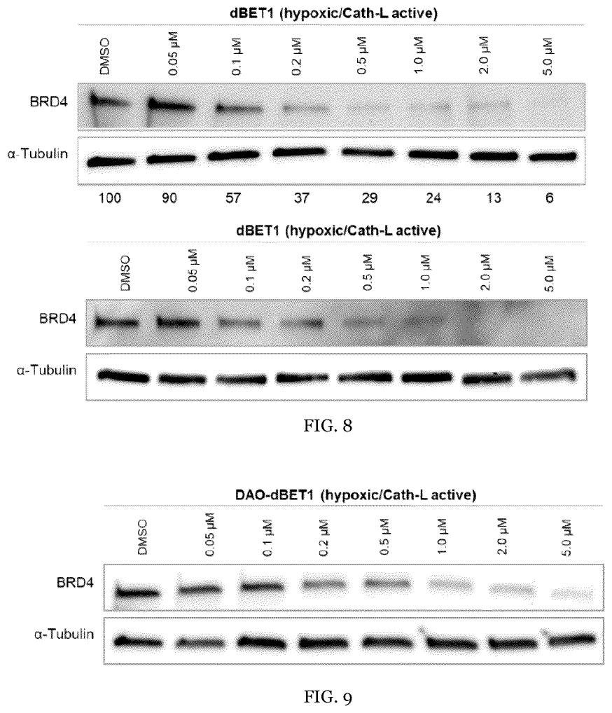

FIG. 16 depicts and provides data regarding a Western blot analysis of BRD4 degradation in HEK-293 cells by treating dBET1 for 24 h as described in the examples.

FIG. 17 depicts and provides data regarding a Western blot analysis of BRD4 degradation by treating DAO-dBET1 for 24 h in HEK-293 cells in presence (left) and absence (right) of 200 μM CoCl₂ solution as described in the examples.

FIGS. 18A and 18B depict quantification of Western blots described above. FIG. 18A provides data regarding the percent BRD4 degradation by treating DAO-dBET1 for 24 h in HEK-293 cells after inducing hypoxic condition using 200 μM CoCl₂ solution and comparison with BRD4 degradation by dBET1 treatment in the same conditions as described in the examples. FIG. 18B provides data regarding the percent BRD4 degradation by treating DAO-dBET1 for 24 h in HEK-293 cells in presence and absence of hypoxia as described in the examples.

FIG. 19 depicts and provides data regarding a Western blot analysis of BRD4 degradation by treating Hyp-dBET1 (left) and Cath-L-dBET1 (right) for 24 h in MDA-MB-231 cells in presence of 200 μM CoCl₂ solution (Cath-L active) as described in the examples.

FIG. 20 depicts and provides data regarding a Western blot analysis of BRD4 degradation by treating Hyp-dBET1(left) and Cath-L-dBET1 (right) for 24 h in MDA-MB-231 cells in absence of hypoxia and Cath-L activity, respectively, as described in the examples.

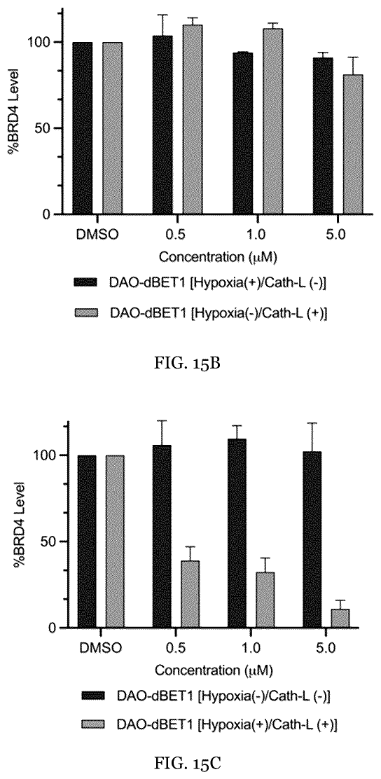

FIG. 21 provides data regarding the change in carbonic anhydrase-9 (CA-9) protein expression using Cath-L-dBET1 and JQ1 in hypoxic/Cath-L inhibited condition in MDA-MB-231 cells as described in the examples.

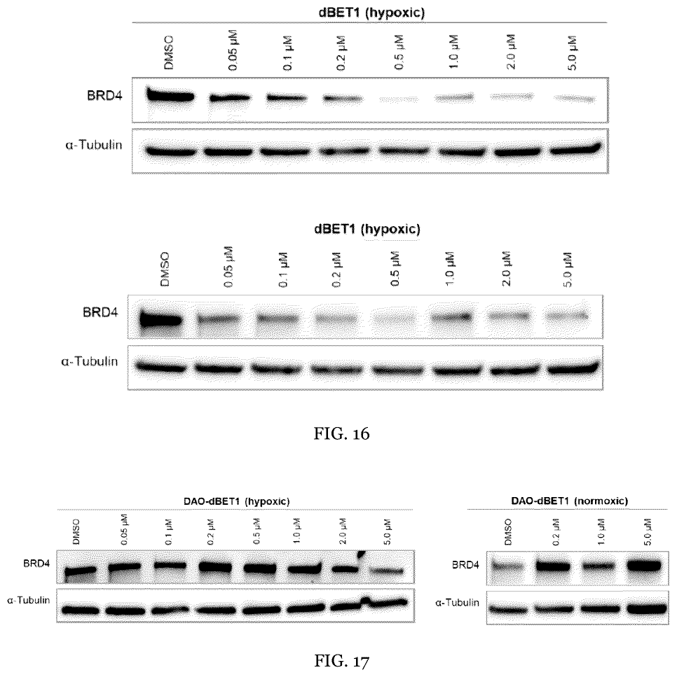

FIG. 22 depicts and provides data regarding Western blot analyses of HIF-1α (left) and Cath-L (right) in HEK-293, MDA-MB-231 and HeLa cell lines as described in the examples.

FIG. 23 provides data regarding a cell viability assay of PROTAC, pro-PROTACs (Hyp-dBET1, Cath-L-dBET1) treatments in MDA-MB-231 cell line after 72 h in presence (left) and absence (right) of corresponding stimuli as described in the examples.

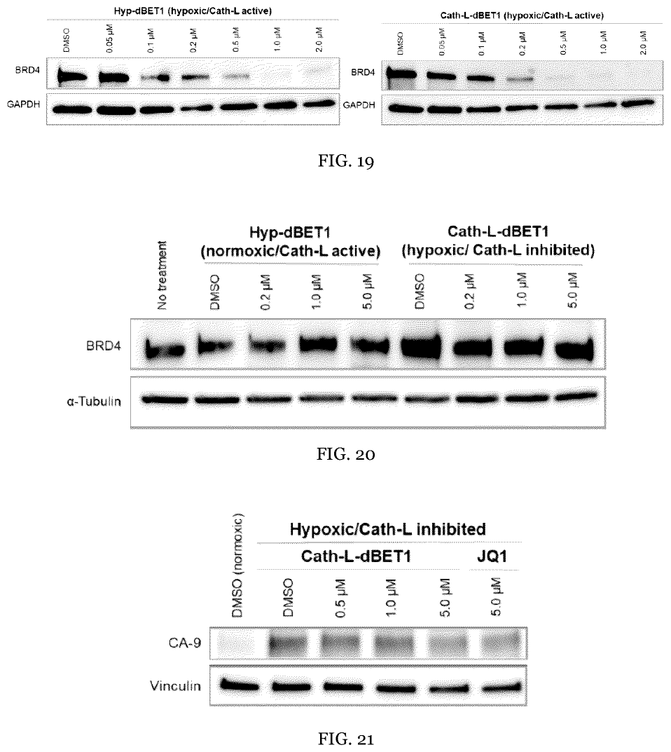

FIG. 24 provides data regarding a cell viability assay of JQ1 and thalidomide-O—COOH treatments in MDA-MB-231 cell line for 72 h in presence of stimuli as described in the examples.

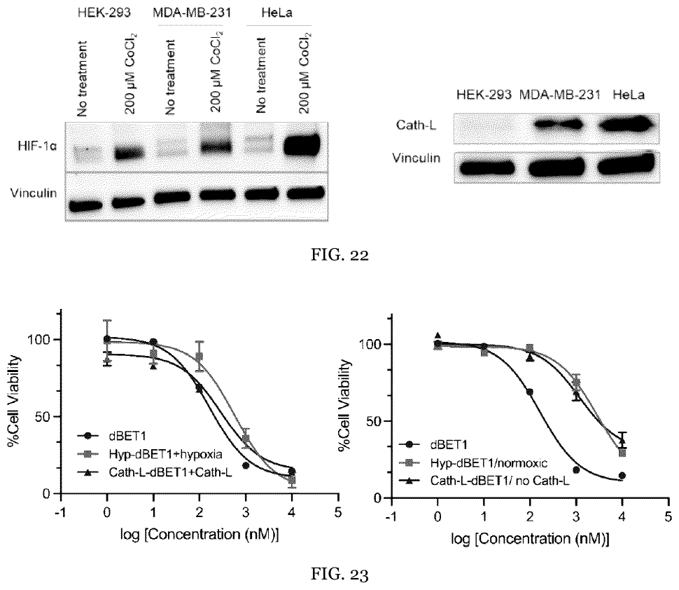

FIG. 25 provides data regarding the investigation of cellular apoptosis using annexin V and propidium iodide (PI) staining assay after treating dBET1 and DAO-dBET1 at 1 μM concentrations in different conditions for 72 h in MDA-MB-231 cells as described in the examples. Lower left quadrant: live cells, lower right quadrant: necrotic cells, upper right quadrant: cells at terminal stage of apoptosis or cell death, upper left quadrant: early apoptotic cells.

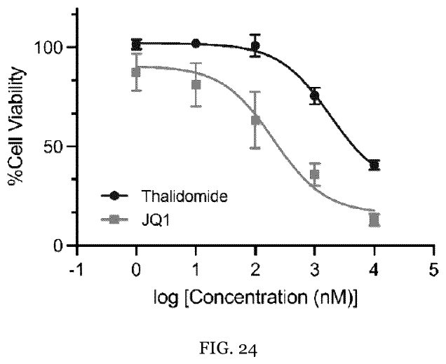

FIG. 26 provides data regarding the investigation of cellular apoptosis using annexin V and PI staining assay after treating dBET1 and DAO-dBET1 at 1 μM concentrations in different conditions for 72 h in HeLa cells as described in the examples. Lower left quadrant: live cells, lower right quadrant: necrotic cells, upper right quadrant: cells at terminal stage of apoptosis or cell death, upper left quadrant: early apoptotic cells.

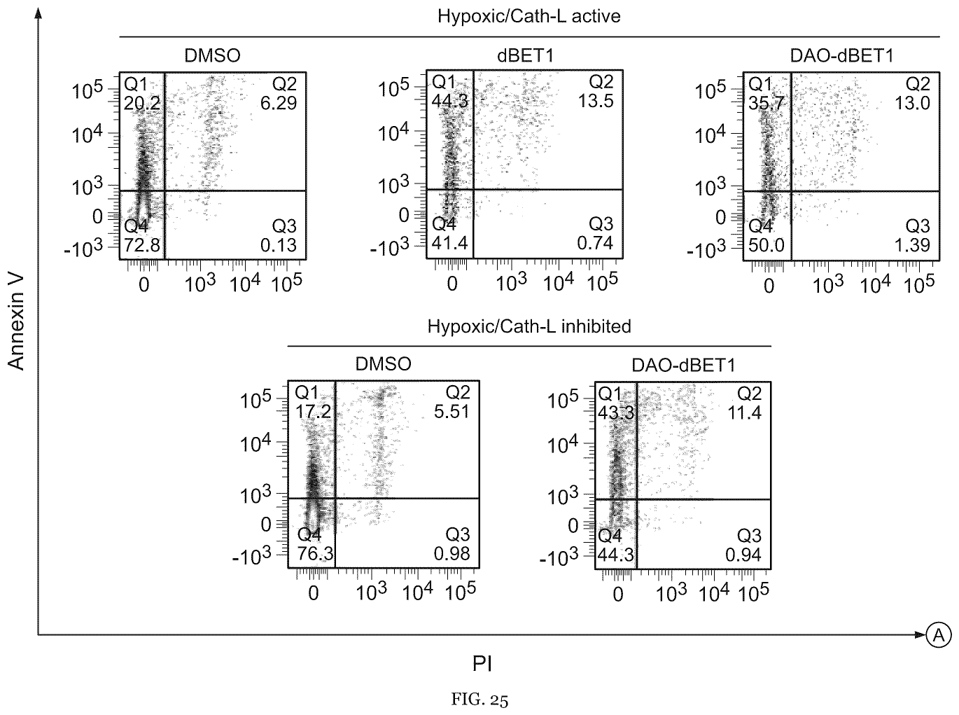

FIG. 27 provides data regarding cell apoptosis assays through caspase 3/7 activity post 72 h treatment of (I) DMSO [Cath-L(+); Hypoxia(+)], (II) dBET1 [Cath-L(+); Hypoxia(+)], (III) DAO-dBET1 [Cath-L(+); Hypoxia(+)], (IV) DMSO [Cath-L(−); Hypoxia(−)], (V) DAO-dBET1 [Cath-L(−); Hypoxia(−)] in MDA-MB-231 cells as described in the examples. Statistical significance calculated by one-way ANOVA. *p<0.1, **p<0.01, ***p<0.001, ****p<0.0001.

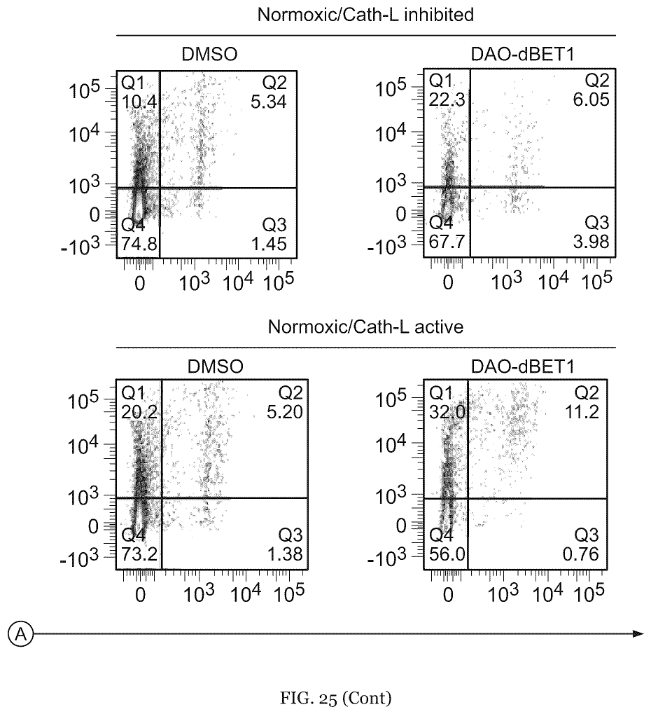

FIG. 28 provides data regarding cell apoptosis assays through caspase 3/7 activity post 72 h treatment of (I) DMSO [Cath-L(+); Hypoxia(+)], (II) dBET1 [Cath-L(+); Hypoxia(+)], (III) DAO-dBET1 [Cath-L(+); Hypoxia(+)], (IV) DMSO [Cath-L(−); Hypoxia(−)], (V) DAO-dBET1 [Cath-L(−); Hypoxia(−)] in HeLa cells as described in the examples. Statistical significance calculated by one-way ANOVA. *p<0.1, **p<0.01, ***p<0.001, ****p<0.0001.

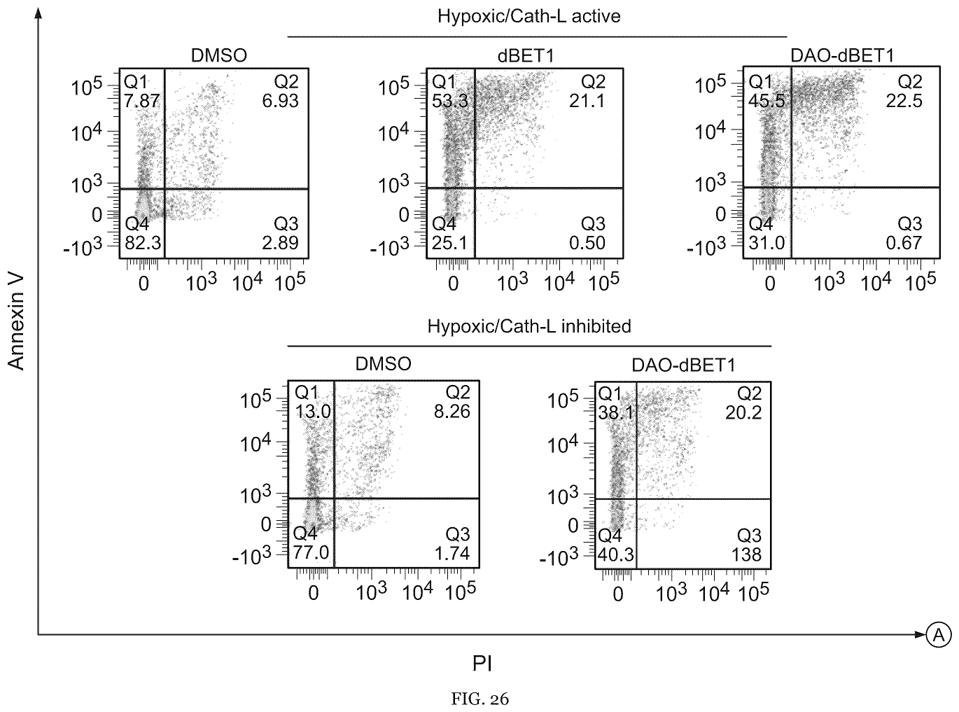

FIG. 29 provides data regarding cell apoptosis assays through caspase 3/7 activity post 72 h treatment of (I) DMSO [Cath-L(−); Hypoxia(+)], (II) DAO-dBET1 [Cath-L(−); Hypoxia(+)], (III) DMSO [Cath-L(+); Hypoxia(−)], (IV) DAO-dBET1 [Cath-L(+); Hypoxia(−)] in HeLa cells as described in the examples. Statistical significance calculated by one-way ANOVA. *p<0.1, **p<0.01, ***p<0.001, ****p<0.0001.

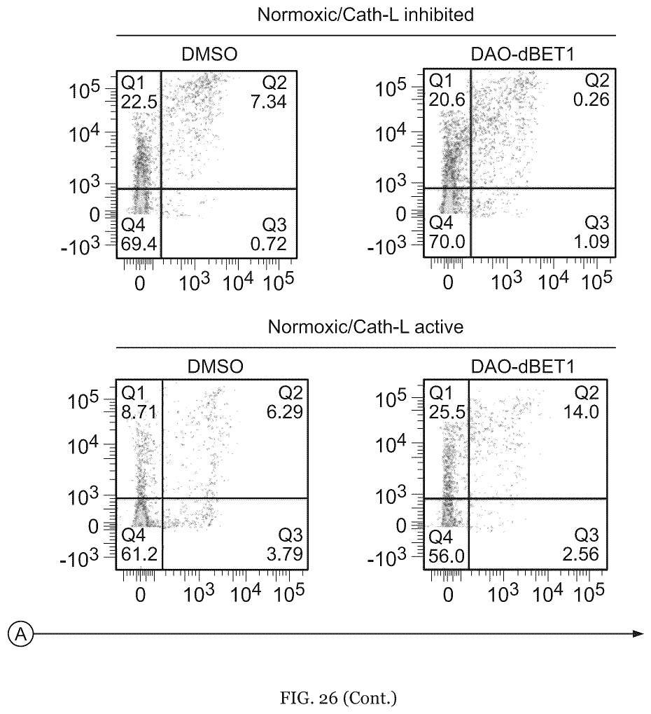

FIGS. 30A-30C depict and provide data regarding cell viability assays for Cath-L-dBET1 (FIG. 30A) in HEK-293 (FIG. 30B) and HeLa (FIG. 30C) cells. Cells were treated with the described molecule for 72 h in the presence and absence of stimuli. The IC50 in HEK-293 cells for dBET1 and Catl-L-dBET1 was 136 nM and 1072 nM, respectively. The IC50 in HeLa cells for dBET1, Cath-L-dBET1 with Cath-L, and Cath-L-dBET1 with Cath-L inhibited was 136 nM, 248 nM, and 426 nM, respectively.

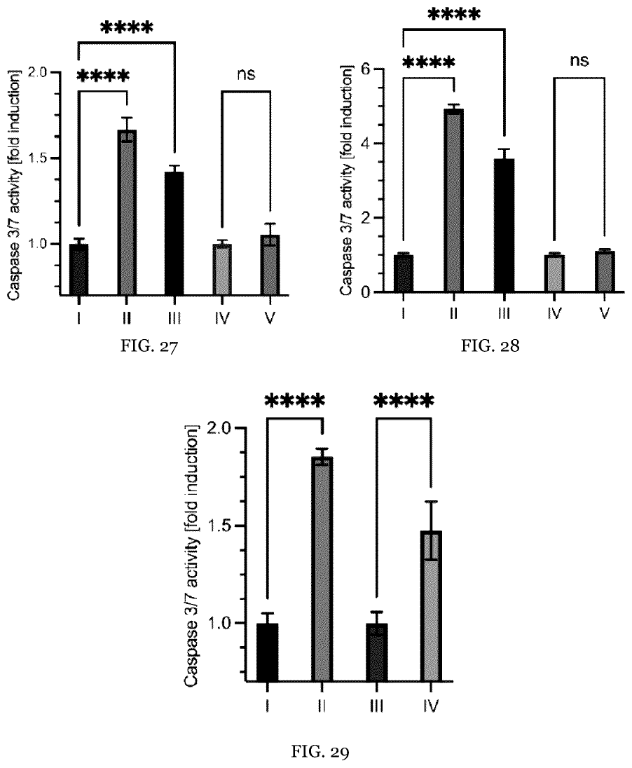

FIGS. 31A-31C depict and provide data regarding cell viability assays for Hyp-dBET1 (FIG. 31A) in HEK-293 (FIG. 31B) and HeLa (FIG. 31C) cells. Cells were treated with the described molecule for 72 h in the presence and absence of stimuli. The IC50 in HEK-293 cells for dBET1, Hyp-dBET1 under hypoxia, and Hyb-dBET1 under normoxia was 72 nM, 153 nM, and 2870 nM, respectively. The IC50 in HeLa cells for dBET1, Hyp-dBET1 under hypoxia, and Hyb-dBET1 under normoxia with Cath-L inhibited was 136 nM, 172 nM, and 3815 nM, respectively.

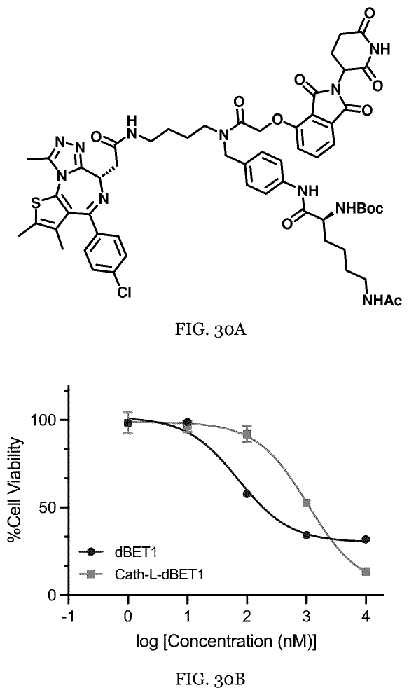

FIGS. 32A-32B depict and provide data regarding the change in c-Myc protein expression using DAO-dBET1 in (FIG. 32A) presence and (FIG. 32B) absence of CoCl₂ (200 μM) in MDA-MB-231 cells after 24 h treatment. For both conditions, cells are cotreated with Z-FY-CHO (100 μM) and the downregulating effect of DAO-dBET1 treatment is compared with JQ1 treatment.

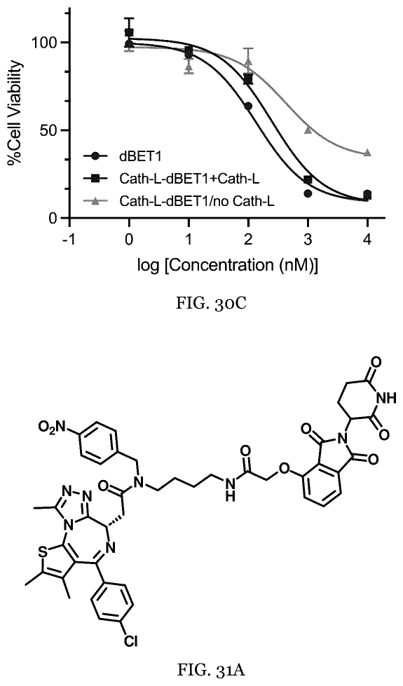

FIGS. 33A-33B depict and provide data regarding the change in c-Myc protein expression using DAO-dBET1 in (FIG. 33A) presence and (FIG. 33B) absence of CoCl₂ (200 μM) in HEK-293 cells after 24 h treatment. The downregulating effect of DAO-dBET1 treatment is compared with JQ1 treatment.

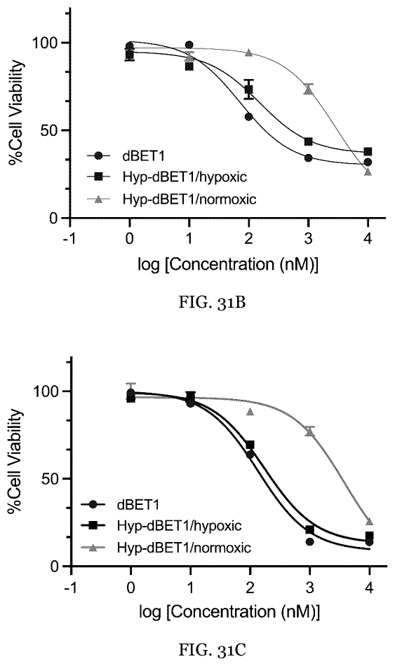

FIG. 34 provides a scheme depicting the synthesis of DAO-THAL-SNS-032.

FIG. 35 depicts Western blot analysis of CDK9 degradation in MDA-MB-231 cells treated with THAL-SNS-032 for 48 h.

FIG. 36 depicts Western blot analysis of CDK9 degradation by treating with DAO-THAL-SNS-032 for 48 h in MDA-MB-231 cells after inducing hypoxic condition using 200 μM CoCl₂ solution (Cathepsin-L active).

FIG. 37 provides data regarding the quantification of Western blots with respect to percent CDK9 degradation by treating DAO-THAL-SNS-032 for 48 h in MDA-MB-231 cells after inducing hypoxic condition using 200 μM CoCl₂ solution (Cath-L active) in comparison with CDK9 degradation by THAL-SNS-032 treatment in the same conditions.

FIGS. 38A-38B depict and provide data regarding (FIG. 38A) Western blot analysis of CDK9 degradation by treating with DAO-THAL-SNS-032 for 48 h in MDA-MB-231 cells in presence of either Cath-L activity or hypoxia and (FIG. 38B) the quantification of Western blots with respect to percent CDK9 degradation by treating with DAO-THAL-SNS-032 for 48 h in MDA-MB-231 cells in presence of either Cath-L activity or hypoxia.

FIGS. 39A-39B depict and provide data regarding (FIG. 39A) Western blot analysis of CDK9 degradation by treating DAO-THAL-SNS-032 for 48 h in MDA-MB-231 cells in absence of both the triggers and (FIG. 39B) the quantification of Western blots with respect to percent CDK9 degradation by treating with DAO-THAL-SNS-032 for 48 h in MDA-MB-231 cells in absence of both the triggers.

FIGS. 40A-40B depict and provide data regarding the change in MCL-1 protein expression using DAO-THAL-SNS-032 in (FIG. 40A) absence and (FIG. 40B) presence of Z-FY-CHO (300 μM) in MDA-MB-231 cells after 48 h treatment. For both conditions, cells are cotreated with CoCl₂ (200 μM) and the downregulating effect of DAO-THAL-SNS-032 treatment is compared with SNS-032 treatment.

FIGS. 41A-41B depict and provide data regarding the change in ZFP-91 protein expression using DAO-THAL-SNS-032 in (FIG. 41A) presence and (FIG. 41B) absence of CoCl₂ (200 μM) in MDA-MB-231 cells after 48 h treatment. For both conditions, cells are cotreated with Z-FY-CHO (300 μM) and the downregulating effect of DAO-THAL-SNS-032 treatment is compared with pomalidomide (Poma) treatment.

FIGS. 42A-42C provide data regarding: (FIGS. 42A-42B) cell viability vs log [Concentration (nM)] of THAL-SNS-032 and DAO-THAL-SNS-032 in presence and absence of hypoxia and Cath-L activity and in the presence of either hypoxia or Cath-L activity in MDA-MB-231 cells (72 h); and (FIG. 42C) Cell viability vs log [Concentration (nM)] of SNS-032 and pomalidomide in presence of hypoxia and Cath-L activity in MDA-MB-231 cells (72 h).

FIGS. 43A-43E depict and provide data regarding an annexin V vs PI Flow Cytometry assay for DAO-THAL-SNS-032 (MDA-MB-231 cells, 72 h, 4 replicates), inducing hypoxia using CoCl₂ (200 μM), inhibiting Cath-L activity using Z-FY-CHO (100 μM) against DMSO controls in similar conditions.

FIGS. 44A-44D depicts and provide data regarding an annexin V vs PI Flow Cytometry assay for DAO-THAL-SNS-032 (MDA-MB-231 cells, 72 h, 4 replicates), inducing hypoxia using CoCl₂ (200 μM), inhibiting Cat-L activity using Z-FY-CHO (100 μM) against DMSO controls in similar conditions.

FIGS. 45A-45B provide data regarding (FIG. 45A) a cell apoptosis assay through annexin V and PI staining using flow cytometry post 72 h treatment of (I) DMSO [Cath-L(+); Hypoxia(+)], (II) THAL-SNS-032 [Cath-L(+); Hypoxia(+)], (III) DAO-THAL-SNS-032 [Cath-L(+); Hypoxia(+)], (IV) DMSO [Cath-L(−); Hypoxia(−)], (V) DAO-THAL-SNS-032 [Cath-L(−); Hypoxia(−)]; and (FIG. 45B) a cell apoptosis assays through annexin V and PI staining through flow cytometry post 72 h treatment of (I) DMSO [Cath-L(+); Hypoxia(−)], (II) DAO-THAL-SNS-032 [Cath-L(+); Hypoxia(−)], (III) DMSO [Cath-L(−); Hypoxia(+)], (IV) DAO-THAL-SNS-032 [Cath-L(−); Hypoxia(+)].

### DETAILED DESCRIPTION

The following description of the disclosure is provided as an enabling teaching of the disclosure in its best, currently known aspects. Many modifications and other aspects disclosed herein will come to mind to one skilled in the art to which the disclosed compositions and methods pertain, benefiting from the teachings presented in the descriptions herein and the associated drawings. Therefore, it is understood that the disclosures are not limited to the specific aspects disclosed and that modifications and other aspects are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As apparent to those of skill in the art upon reading this disclosure, each of the individual aspects described and illustrated herein has discrete components and features that may be readily separated from or combined with the features of any of the other several aspects without departing from the scope or spirit of the present disclosure.

Any recited method can be carried out in the order of events recited or any other order that is logically possible. Unless otherwise expressly stated, it is in no way intended that any method or aspect set forth herein be construed as requiring that its steps be performed in a specific order. Accordingly, where a method claim does not explicitly state in the claims or descriptions that the steps are to be limited to a particular order, it is in no way intended that an order be inferred in any respect. This holds for any possible non-express basis for interpretation, including logic concerning arrangement of steps or operational flow, meaning derived from grammatical organization or punctuation, or the number or type of aspects described in the specification.

All publications mentioned herein are incorporated by reference to disclose and describe the methods or materials in connection with which the publications are cited. The publications discussed herein are provided solely for their disclosure before the filing date of the present application. Further, the dates of publication provided herein can be different from the actual publication dates, which can require independent confirmation.

It is also to be understood that the terminology herein describes particular aspects only and is not intended to be limiting. Unless defined otherwise, all technical and scientific terms herein have the same meaning as commonly understood by one of ordinary skill in the art to which the disclosed compositions and methods belong. It can be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Before describing the various aspects of the present disclosure, the following definitions are provided and should be used unless otherwise indicated. Additional terms may be defined elsewhere in the present disclosure.

### Definitions

As used herein, “comprising” is interpreted as specifying the presence of the stated features, integers, steps, or components but does not preclude the presence or addition of one or more features, integers, steps, components, or groups thereof. Moreover, each of the terms “by,” “comprising,” “comprises,” “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, “consisting essentially of” is intended to include examples encompassed by the term “consisting of.”

As used in the specification and the appended claims, the singular forms “a,” “an,” and “the” include plural referents unless the context dictates otherwise.

Ratios, concentrations, amounts, and other numerical data can be expressed herein in a range format. Further, the endpoints of each of the ranges are significant both in relation to the other endpoint and independently of the other endpoint. There are many values disclosed herein, and each value is also disclosed as “about” that particular value in addition to the value itself. For example, if the value “10” is disclosed, then “about 10” is also disclosed. Ranges can be expressed herein as from “about” one particular value and to “about” another particular value. Similarly, when values are expressed as approximations, using the antecedent “about,” the particular value forms a further aspect. For example, if the value “about 10” is disclosed, then “10” is also disclosed.

When a range is expressed, a further aspect includes from the one particular value and to the other particular value. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g. ‘about x, y, z, or less' and should be interpreted to include the specific ranges of ‘about x,’ ‘about y,’ and ‘about z’ as well as the ranges of ‘less than x,’ ‘less than y.’ and ‘less than z.’ Likewise, the phrase ‘about x, y, z, or greater’ should be interpreted to include the specific ranges of ‘about x,’ ‘about y,’ and ‘about z’ as well as the ranges of ‘greater than x,’ greater than y,’ and ‘greater than z.’ In addition, the phrase “about ‘x’ to ‘y’,” where ‘x’ and ‘y’ are numerical values, includes “about ‘x’ to about ‘y’.”

Such a range format is used for convenience and brevity and thus, should be interpreted flexibly to include not only the numerical values explicitly recited as the limits of the range but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, amounts, sizes, formulations, parameters, and other quantities and characteristics are not and need not be exact but may be approximate, larger or smaller, as desired, reflecting tolerances, conversion factors, rounding, measurement error, and the like, and other factors known to those of skill in the art such that equivalent results or effects are obtained. In some circumstances, the value that provides equivalent results or effects cannot be reasonably determined. In such cases, as used herein, “about” and “at or about” mean the nominal value indicated ±10% variation unless otherwise indicated or inferred. In general, an amount, size, formulation, parameter, or other quantity or characteristic is “about,” “approximate,” or “at or about,” whether or not expressly stated to be such. Where “about,” “approximate,” or “at or about” is used before a quantitative value, the parameter also includes the specific quantitative value itself unless expressly stated otherwise.

As used herein, the term “therapeutically effective amount” refers to an amount sufficient to achieve the desired therapeutic result or to have an effect on undesired symptoms but generally insufficient to cause adverse side effects. The specific therapeutically effective dose level for any particular patient will depend upon a variety of factors, including the disorder being treated and the severity of the disorder; the specific composition employed; the age, body weight, general health, sex, and diet of the patient; the time of administration; the route of administration; the rate of excretion of the specific compound employed; the duration of the treatment; drugs used in combination or coincidental with the particular compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to permanently halt the progression of the disease. This can be monitored by routine diagnostic methods known to one of ordinary skill in the art for any particular disease. The desired response to treatment of the disease or condition can also be delaying the onset or even preventing the onset.

For example, it is well within the skill of the art to start doses of a compound at levels lower than those required to achieve the desired therapeutic effect and to increase the dosage gradually until the desired effect is achieved. If desired, the effective daily dose can be divided into multiple doses for administration. Consequently, single dose compositions can contain such amounts or submultiples thereof to make up the daily dose. The individual physician can adjust the dosage in the event of any contraindications. It is generally preferred that a maximum dose of the pharmacological agents of the disclosure (alone or in combination with other therapeutic agents) be used, that is, the highest safe dose according to sound medical judgment. However, a patient may insist on a lower or tolerable dose for medical reasons, psychological reasons, or virtually any other reason.

A response to a therapeutically effective dose of a disclosed compound or composition can be measured by determining the physiological effects of the treatment or medication, such as the decrease or lack of disease symptoms following the administration of the treatment or pharmacological agent. Other assays will be known to one of ordinary skill in the art and can be employed for measuring the level of the response. The amount of a treatment may be varied, for example, by increasing or decreasing the amount of a disclosed compound or pharmaceutical composition, changing the disclosed compound or pharmaceutical composition administered, changing the route of administration, changing the dosage timing, and so on. Dosage can vary and can be administered in one or more dose administrations daily for one or several days. Guidance can be found in the literature for appropriate dosages for given classes of pharmaceutical products.

As used herein, “optional” or “optionally” means that the subsequently described event or circumstance can or cannot occur. The description includes instances where said event or circumstance occurs and those where it does not.

As used interchangeably herein, “subject,” “individual,” or “patient” can refer to a vertebrate organism, such as a mammal (e.g., human). “Subject” can also refer to a cell, a population of cells, a tissue, an organ, or an organism, preferably to a human and constituents thereof.

As used herein, “treating” and “treatment” generally refer to obtaining a desired pharmacological or physiological effect. The effect can be but does not necessarily have to be prophylactic in preventing or partially preventing a disease, symptom, or condition. The effect can be therapeutic regarding a partial or complete cure of a disease, condition, symptom, or adverse effect attributed to the disease, disorder, or condition. The term “treatment” as used herein can include any treatment of a disorder in a subject, particularly a human. It can include any one or more of the following: (a) preventing the disease from occurring in a subject who may be predisposed to the disease but has not yet been diagnosed as having it; (b) inhibiting the disease, i.e., arresting its development; and (c) relieving the disease, i.e., mitigating or ameliorating the disease or its symptoms or conditions. The term “treatment,” as used herein, can refer to both therapeutic treatment alone, prophylactic treatment alone, or both therapeutic and prophylactic treatment. Those in need of treatment (i.e., subjects in need thereof) can include those already with the disorder or those in which the disorder is to be prevented. As used herein, the term “treating” can include inhibiting the disease, disorder, or condition, e.g., impeding its progress; and relieving the disease, disorder, or condition, e.g., causing regression of the disease, disorder, or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “dose,” “unit dose,” or “dosage” can refer to physically discrete units suitable for use in a subject, each unit containing a predetermined quantity of a disclosed compound or a pharmaceutical composition thereof calculated to produce the desired response or responses in association with its administration.

As used herein, “therapeutic” can refer to treating, healing, or ameliorating a disease, disorder, condition, or side effect, or decreasing the rate of advancement of a disease, disorder, condition, or side effect.

As used herein, the term or phrase “effective,” “effective amount,” or “conditions effective to” refers to such amount or condition that is capable of performing the function or property for which an effective amount or condition is expressed. As will be pointed out below, the exact amount or particular condition required will vary from one aspect to another, depending on recognized variables such as the materials employed and the processing conditions observed. Thus, it is not always possible to specify an exact “effective amount” or “condition effective to.” However, it should be understood that an appropriate effective amount will be readily

It will be understood that, although the terms “first,” “second,” etc., can be used herein to describe various elements, these elements should not be limited by these terms. These terms are only used to distinguish one element from another element. Thus, a first element discussed herein could be termed a second element without departing from the teachings of various aspects.

As used herein, the term “substantially” means that the subsequently described event or circumstance completely occurs or that the subsequently described event or circumstance generally, typically, or approximately occurs.

Still further, the term “substantially” can, in some aspects, refer to at least about 90%, at least about 91%, at least about 92%, at least about 93%, at least about 94%, at least about 95%, at least about 96%, at least about 97%, at least about 98%, at least about 99%, or about 100% of the stated property, component, composition, or other condition for which substantially is used to characterize or otherwise quantify an amount.

### Chemical Definitions

Compounds are described using standard nomenclature. Unless defined otherwise, all technical and scientific terms used herein have the same meaning as is commonly understood by one of skill in the art to which this disclosure belongs.

The compounds described herein include enantiomers, mixtures of enantiomers, diastereomers, tautomers, racemates, and other isomers, such as rotamers, as if each is specifically described unless otherwise indicated or otherwise excluded by context. It is to be understood that the compounds provided herein may contain chiral centers. Such chiral centers may be of either the (R) or (S) configuration. The compounds provided herein may either be enantiomerically pure or be diastereomeric or enantiomeric mixtures. It is to be understood that the chiral centers of the compounds provided herein may undergo epimerization in vivo. As such, one of ordinary skill in the art will recognize that administering a compound in its (R) form is equivalent, for compounds that undergo epimerization in vivo, to administering the compound in its (S) form. Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

Compounds described herein may contain one or more double bonds and, thus, potentially give rise to cis/trans (E/Z) isomers, as well as other conformational isomers unless stated to the contrary, all such possible isomers are contemplated, as well as mixtures of such isomers.

Compounds described herein may also be present as an equilibrium of tautomers. For example, ketones with an α-hydrogen can exist in an equilibrium of the keto form and the enol form. Likewise, amides with an N-hydrogen can exist in an equilibrium of the amide form and the imidic acid form. Unless stated to the contrary, all possible tautomers of the compounds described herein are contemplated.

A dash (“-”) that is not between two letters or symbols indicates a point of attachment for a substituent. For example, —(C═O)NH₂ is attached through the carbon of the keto (C═O) group.

The term “substituted,” as used herein, means that any one or more hydrogens on the designated atom or group are replaced with a moiety selected from the indicated group, provided that the designated atom's normal valence is not exceeded and the resulting compound is stable. For example, when the substituent is oxo (i.e., ═O), two hydrogens on the atom are replaced. For example, a pyridyl group substituted by oxo is a pyridine. Combinations of substituents and/or variables are permissible only if such combinations result in stable compounds or useful synthetic intermediates. A stable active compound refers to a compound that can be isolated and/or can be formulated into a form with a shelf life of at least one month. A stable manufacturing intermediate or precursor to an active compound is stable if it does not degrade within the period needed for reaction or other use. A stable moiety or substituent group is one that does not degrade, react, or fall apart within the period necessary for use. Non-limiting examples of unstable moieties are those that combine heteroatoms in an unstable arrangement, as typically known and identifiable to those of skill in the art.

Any suitable group may be present on a “substituted” or “optionally substituted” position that forms a stable molecule and meets the desired purpose of the disclosure and includes, but is not limited to: halo, nitro, cyano, azido, oxo, C₁-C₆ alkyl, C₁-C₆ haloalkyl, C₂-C₆ alkenyl, C₂-C₆ alkynyl, (C₃-C₇ cycloalkyl)(C₀-C₃ alkyl)-, (3- to 8-membered monocyclic or bicyclic heterocycle)-(C₀-C₆ alkyl)-, (6- to 10-membered monocyclic or bicyclic aryl)-(C₀-C₆ alkyl)-, (5- to 10-membered monocyclic or bicyclic heteroaryl)-(C₀-C₆ alkyl)-, A^{x}O—(C₀-C₆ alkyl)-, A^{x}S—(C₀-C₆ alkyl)-, (A^{x}A^{y}N)—(C₀-C₆ alkyl)-, A^{z}C(O)—(C₀-C₆ alkyl)-, A^{z}C(N)—(C₀-C₆ alkyl)-, and A^{z}S(O)—(C₀-C₆ alkyl)-, and A^{z}S(O)₂—(C₀-C₆ alkyl)-, wherein A^{x} and A^{y} are independently selected at each occurrence from A^{a}, A^{z}C(O)—, A^{z}C(N)—, A^{z}S(O)—, and A^{z}S(O)₂—, each of which may be optionally substituted with one or more B groups as allowed by valency; wherein A^{z} is independently selected at each occurrence from hydrogen, halo, C₁-C₆alkyl, C₁-C₆haloalkyl, C₂-C₆alkenyl, C₂-C₆alkynyl, (C₃-C₇ cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, —OA^{a}, —SA^{a}, and —NA^{a}A^{b}, each of which may be optionally substituted with one or more B groups as allowed by valency; wherein A^{a} and A^{b} are independently selected at each occurrence from hydrogen, C₁-C₆alkyl, C₁-C₆haloalkyl, C₂-C₆alkenyl, C₂-C₆alkynyl, (C₃-C₇ cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, each of which may be optionally substituted by one or more B groups as allowed by valency; and wherein B is independently selected at each occurrence from hydrogen, halo, nitro, cyano, azido, oxo, C₁-C₆ alkyl, C₁-C₆ haloalkyl, C₂-C₆ alkenyl, C₂-C₆ alkynyl, (C₃-C₇ cycloalkyl)(C₀-C₃ alkyl)-, (3- to 8-membered monocyclic or bicyclic heterocycle)-(C₀-C₆ alkyl)-, (6- to 10-membered monocyclic or bicyclic aryl)-(C₀-C₆ alkyl)-, (5- to 10-membered monocyclic or bicyclic heteroaryl)-(C₀-C₆ alkyl)-, A^{p}O—, A^{p}S—, A^{p}A^{q}N—, A^{o}C(O)—, A^{o}C(O)—O—, A^{o}C(O)—NA^{q}-, A^{o}S(O)₂—, A^{o}S(O)₂—O—, and A^{o}S(O)₂—NA^{q}-, wherein A^{o} is independently selected at each occurrence from A^{p}, halo, A^{p}O—, and A^{p}A^{q}N—, and wherein A^{p} and A^{q} are independently selected at each occurrence from hydrogen, C₁-C₆ alkyl, C₁-C₆ haloalkyl, C₂-C₆ alkenyl, C₂-C₆ alkynyl, (C₃-C₇ cycloalkyl)(C₀-C₃ alkyl)-, (3- to 8-membered monocyclic or bicyclic heterocycle)-(C₀-C₆ alkyl)-, (6- to 10-membered monocyclic or bicyclic aryl)-(C₀-C₆ alkyl)-, and (5- to 10-membered monocyclic or bicyclic heteroaryl)-(C₀-C₆ alkyl)-.

The terms for various functional groups as used herein are not intended to be limited to monovalent radicals and may include polyvalent radical groups as appropriate, such as divalent, trivalent, tetravalent, pentavalent, and hexavalent groups, and the like, based on the position and location of such groups in the compounds described herein as would be readily understood by the skilled person in the context in which said functional groups are recited.

As used herein, the symbol

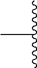

(which hereinafter can be referred to as “a point of attachment bond”) denotes a bond that is a point of attachment between two chemical entities, one of which is depicted as being attached to the point of attachment bond and the other of which is not depicted as being attached to the point of attachment bond. For example,

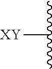

indicates that the chemical entity “XY” is bonded to another chemical entity via the point of attachment bond. Furthermore, the specific point of attachment to the non-depicted chemical entity can be specified by inference. For example, the compound CH₃—R³, wherein R³ is H or

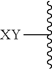

infers that when R³ is “XY”, the point of attachment bond is the same bond as the bond by which R³ is depicted as being bonded to CH₃.

“Halo” or “halogen” independently indicates any fluoro, chloro, bromo, or iodo.

The term “nitro,” as used herein, is represented by the formula —NO₂.

The term “cyano,” as used herein, is represented by the formula —CN

The term “azido,” as used herein, is represented by the formula —N₃.

The term “oxo,” as used herein, is represented by the formula ═O.

“Alkyl” is a straight chain or branched saturated aliphatic hydrocarbon group. In certain aspects, the alkyl is C₁-C₂, C₁-C₃, or C₁-C₆ (i.e., the alkyl chain can be 1, 2, 3, 4, 5, or 6 carbons in length). The specified ranges as used herein indicate an alkyl group with the length of each member of the range described as an independent species. For example, C₁-C₆alkyl, as used herein, indicates an alkyl group having 1, 2, 3, 4, 5, or 6 carbon atoms and is intended to mean that each of these is described as an independent species, and C₁-C₄alkyl, as used herein, indicates an alkyl group having 1, 2, 3, or 4 carbon atoms and is intended to mean that each of these is described as an independent species. When C₀-Cₙalkyl is used herein in conjunction with another group, for example (C₃-C₇ cycloalkyl)C₀-C₄alkyl, or —C₀-C₄(C₃-C₇ cycloalkyl), the indicated group, in this case cycloalkyl, is either directly bound by a single covalent bond (C₀alkyl), or attached by an alkyl chain, in this case 1, 2, 3, or 4 carbon atoms. Alkyls can also be attached via other groups such as heteroatoms, such as —O—C₀-C₄alkyl(C₃-C₇ cycloalkyl). Examples of alkyl include but are not limited to, methyl, ethyl, n-propyl, isopropyl, n-butyl, isobutyl, sec-butyl, t-butyl, n-pentyl, isopentyl, tert-pentyl, neopentyl, n-hexyl, 2-methylpentane, 3-methylpentane, 2,2-dimethylbutane, and 2,3-dimethylbutane. In some aspects, the alkyl group is optionally substituted as described herein.

“Haloalkyl” refers to an alkyl group that is substituted with one or more halo groups, e.g., fluoro, chloro, bromo, iodo, or combinations thereof.

“Cycloalkyl” is a saturated or partially unsaturated mono- or multicyclic hydrocarbon ring system. When composed of two or more rings, the rings may be joined together in a fused or bridged fashion. Non-limiting examples of typical cycloalkyl groups include cyclopropyl, cyclobutyl, cyclopentyl, cyclohexyl, and cycloheptyl. In some aspects, the cycloalkyl group is optionally substituted as described herein.

“Alkenyl” is a straight or branched chain aliphatic hydrocarbon group having one or more carbon-carbon double bonds, each of which is independently either cis or trans, that may occur at a stable point along the chain. Non-limiting examples include C₂-C₄alkenyl and C₂-C₆alkenyl (i.e., having 2, 3, 4, 5, or 6 carbons). The specified ranges as used herein indicate an alkenyl group, with each member of the range described as an independent species, as described above for the alkyl moiety. Examples of alkenyl include, but are not limited to, ethenyl and propenyl. In one aspect, the alkenyl group is optionally substituted as described herein.

“Alkynyl” is a straight or branched chain aliphatic hydrocarbon group having one or more carbon-carbon triple bonds that may occur at any stable point along the chain, for example, C₂-C₄alkynyl or C₂-C₆alkynyl (i.e., having 2, 3, 4, 5, or 6 carbons). The specified ranges as used herein indicate an alkynyl group, with each member of the range described as an independent species, as described above for the alkyl moiety. Examples of alkynyl include, but are not limited to, ethynyl, propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, and 5-hexynyl. In one aspect, the alkynyl group is optionally substituted as described herein.

“Aryl” indicates an aromatic group containing only carbon in the aromatic ring or rings. In one aspect, the aryl group contains 1 to 3 separate or fused rings and is 6 to 14 or 18 ring atoms, without heteroatoms as ring members. When indicated, such aryl groups may be further substituted with carbon or non-carbon atoms or groups. Such substitution may include the fusion to a 4- to 7- or 5- to 7-membered saturated or partially unsaturated cyclic group that optionally contains 1, 2, or 3 heteroatoms independently selected from N, O, B, P, Si, and S, to form, for example, a 3,4-methylenedioxyphenyl group. Aryl groups include, for example, phenyl and naphthyl, including 1-naphthyl and 2-naphthyl. In one aspect, aryl groups are pendant. An example of a pendant ring is a phenyl group substituted with a phenyl group. In one aspect, the aryl group is optionally substituted as described herein.

The term “heterocycle” refers to saturated and partially saturated heteroatom-containing ring radicals, where the heteroatoms may be selected from N, O, and S. The term heterocycle includes monocyclic 3-12 members rings, as well as bicyclic 5-16 membered ring systems (which can include fused, bridged, or spiro bicyclic ring systems). It does not include rings containing —O—O—, —O—S—, and —S—S— portions. Examples of saturated heterocycle groups, including saturated 4- to 7-membered monocyclic groups containing 1 to 4 nitrogen atoms [e.g., pyrrolidinyl, imidazolidinyl, piperidinyl, pyrrolinyl, azetidinyl, piperazinyl, and pyrazolidinyl]; saturated 4- to 6-membered monocyclic groups containing 1 to 2 oxygen atoms and 1 to 3 nitrogen atoms [e.g., morpholinyl]; and saturated 3- to 6-membered heteromonocyclic groups containing 1 to 2 sulfur atoms and 1 to 3 nitrogen atoms [e.g., thiazolidinyl]. Examples of partially saturated heterocycle radicals include, but are not limited to, dihydrothienyl, dihydropyranyl, dihydrofuryl, and dihydrothiazolyl. Examples of partially saturated and saturated heterocycle groups include, but are not limited to, pyrrolidinyl, imidazolidinyl, piperidinyl, pyrrolinyl, pyrazolidinyl, piperazinyl, morpholinyl, tetrahydropyranyl, thiazolidinyl, dihydrothienyl, 2,3-dihydro-benzo[1,4]dioxanyl, indolinyl, isoindolinyl, dihydrobenzothienyl, dihydrobenzofuryl, isochromanyl, chromanyl, 1,2-dihydroquinolyl, 1,2,3,4-tetrahydro-isoquinolyl, 1,2,3,4-tetrahydro-quinolyl, 2,3,4,4a,9,9a-hexahydro-1H-3-aza-fluorenyl, 5,6,7-trihydro-1,2,4-triazolo[3,4-a]isoquinolyl, 3,4-dihydro-2H-benzo[1,4]oxazinyl, benzo[1,4]dioxanyl, 2,3,-dihydro-1H-benzo[d]isothazol-6-yl, dihydropyranyl, dihydrofuryl, and dihydrothiazolyl. Bicyclic heterocycle includes groups wherein the heterocyclic radical is fused with an aryl radical, and the point of attachment is the heterocycle ring. Bicyclic heterocycle also includes heterocyclic radicals that are fused with a carbocyclic radical. Representative examples include but are not limited to, partially unsaturated condensed heterocyclic groups containing 1 to 5 nitrogen atoms, for example, indoline and isoindoline, partially unsaturated condensed heterocyclic groups containing 1 to 2 oxygen atoms and 1 to 3 nitrogen atoms, partially unsaturated condensed heterocyclic groups containing 1 to 2 sulfur atoms and 1 to 3 nitrogen atoms, and saturated condensed heterocyclic groups containing 1 to 2 oxygen or sulfur atoms. In one aspect, the heterocycle group is optionally substituted as described herein.

“Heteroaryl” refers to a stable monocyclic, bicyclic, or multicyclic aromatic ring that contains from 1 to 4, or in some aspects 1, 2, or 3 heteroatoms selected from N, O, S, B, and P (and typically selected from N, O, and S) with remaining ring atoms being carbon, or a stable bicyclic or tricyclic system containing at least one 5, 6, or 7 membered aromatic ring which contains from 1 to 4, or in some aspects from 1 to 3 or from 1 to 2, heteroatoms selected from N, O, S, B, or P, with remaining ring atoms being carbon. In one aspect, the only heteroatom is nitrogen. In one aspect, the only heteroatom is oxygen. In one aspect, the only heteroatom is sulfur. Monocyclic heteroaryl groups typically have from 5 to 6 ring atoms. In some aspects, bicyclic heteroaryl groups are 8- to 10-membered heteroaryl groups, that is groups containing 8 or 10 ring atoms in which one 5-, 6-, or 7-membered aromatic ring which contains from 1 to 4 heteroatoms selected from N, O, S, B, or P is fused to a second aromatic or non-aromatic ring, wherein the point of attachment is an aromatic ring. When the total number of S and O atoms in the heteroaryl ring exceeds 1, these heteroatoms are not adjacent to one another within the ring. In one aspect, the total number of S and O atoms in the heteroaryl ring is not more than 2. In another aspect, the total number of S and O atoms in the heteroaryl ring is not more than 1. Examples of heteroaryl groups include, but are not limited to, pyridinyl, imidazolyl, imidazopyridinyl, pyrimidinyl, pyrazolyl, triazolyl, pyrazinyl, furyl, thienyl, isoxazolyl, thiazolyl, oxadiazolyl, oxazolyl, isothiazolyl, pyrrolyl, quinolinyl, isoquinolinyl, tetrahydroisoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, triazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. In one aspect, the heteroaryl group is optionally substituted as described herein.

A “pharmaceutically acceptable salt” is a derivative of the disclosed compound in which the parent compound is modified by making inorganic and organic, pharmaceutically acceptable, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like) or by reacting free base forms of these compounds with a stoichiometric amount of the appropriate acid. Such reactions are typically carried out in water, an organic solvent, or in a mixture of the two. Generally, non-aqueous media like ether, ethyl acetate, ethanol, isopropanol, or acetonitrile are typical, where practicable. Salts of the present compounds further include solvates of the compounds and of the compound salts. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include salts which are acceptable for human consumption and the quaternary ammonium salts of the parent compound formed, for example, from inorganic or organic salts. Example of such salts include, but are not limited to, those derived from inorganic acids such as hydrochloric, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicyclic, mesylic, esylic, besylic, sulfanilic, 2-acetoxybenzoic, fumaric, toluenesulfonic, methanesulfonic, ethane disulfonic, oxalic, isethionic, HOOC—(CH₂)₁₋₄—COOH, and the like, or using a different acid that produced the same counterion. Suitable counterions found in pharmaceutically acceptable salts described herein include, but are not limited to, cations such as calcium, chloroprocaine, choline, diethanolamine, ethanolamine, ethylenediamine, meglumine, potassium, procaine, sodium, triethylamine, and zinc, and anions such as acetate, aspartate, benzenesulfonate, besylate, bicarbonate, bitartrate, bromide, camsylate, carbonate, chloride, citrate, decanoate, edetate, esylate, fumarate, gluceptate, gluconate, glutamate, glycolate, hexanoate, hydroxynaphthoate, iodide, isethionate, lactate, lactobionate, malate, maleate, mandelate, mesylate, methylsulfate, mucate, napsylate, nitrate, octanoate, oleate, pamoate, pantothenate, phosphate, polygalacturonate, propionate, salicylate, stearate, succinate, sulfate, tartrate, teoclate, and tosylate. Lists of additional suitable salts may be found, e.g., in Remington's Pharmaceutical Sciences, 17^{th} ed., Mack Publishing Company, Easton, PA., p. 1418 (1985).

As used herein, the term “derivative” refers to a compound having a structure derived from the structure of a parent compound (e.g., a compound disclosed herein) and whose structure is sufficiently similar to those disclosed herein and based upon that similarity would be expected by one skilled in the art to exhibit the same or similar activities and utilities as the claimed compounds, or to induce, as a precursor, the same or similar activities and utilities as the claimed compound. Exemplary derivatives include but are not limited to, salts, esters, amides, salts of esters or amides, and N-oxides of a parent compound.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers, such as Sigma-Aldrich (formally MilliporeSigma, Burlington, MA) or Thermo Fisher Scientific Inc. (Waltham, MA), or are prepared by methods known to those skilled in the art following procedures set forth in references such as Fieser and Fieser's Reagents for Organic Synthesis (John Wiley and Sons, 2007); Organic Reactions (John Wiley and Sons, 2004); March's Advanced Organic Chemistry, (John Wiley and Sons, 8^{th} Edition); and Larock's Comprehensive Organic Transformations (John Wiley and Sons, 3^{rd} edition, 2017).

The present disclosure also includes compounds described herein with at least one desired isotopic substitution of an atom at an amount above the natural abundance of the isotope, i.e., enriched.

Examples of isotopes that can be incorporated into compounds of the present disclosure include isotopes of hydrogen, carbon, nitrogen, oxygen, phosphorous, fluorine, and chlorine, such as ²H, ³H, ¹¹C, ¹³C, ¹⁵N, ¹⁷O, ¹⁸O, ¹⁸F, ³¹P, ³²P, ³⁵S, ³⁶Cl, and ¹²⁵I, respectively. In one aspect, isotopically labeled compounds can be used in metabolic studies (with ¹⁴C), reaction kinetic studies (with, for example, ²H or ³H), detection or imaging techniques, such as positron emission tomography (PET) or single-photon emission computed tomography (SPECT) including drug and substrate tissue distribution assays, or in radioactive treatment of patients. In particular, an ¹⁸F-labeled compound may be particularly desirable for PET or SPECT studies. Isotopically labeled compounds of this disclosure and prodrugs thereof can generally be prepared by carrying out the procedures disclosed herein by substituting a readily available isotopically labeled reagent for a non-isotopically labeled reagent.

Byway of general example and without limitation, isotopes of hydrogen, for example, deuterium (²H) and tritium (³H), may optionally be used anywhere in described structures that achieve the desired result. Alternatively, or in addition, isotopes of carbon, e.g., ¹³C and ¹⁴C, may be used. In one aspect, the isotopic substitution is replacing hydrogen with deuterium at one or more locations on the molecule to improve the performance of the molecule as a drug, for example, the pharmacodynamics, pharmacokinetics, biodistribution, half-life, stability, AUC, Tₘₐₓ, Cₘₐₓ, etc. For example, the deuterium can be bound to carbon in the allocation of bond breakage during metabolism (an alpha-deuterium kinetic isotope effect) or next to or near the site of bond breakage (a beta-deuterium kinetic isotope effect).

Isotopic substitutions, for example, deuterium substitutions, can be partial or complete. Partial deuterium substitution means that at least one hydrogen is substituted with deuterium. In certain aspects, the isotope is 80, 85, 90, 95, or 99% or more enriched in an isotope at any location of interest. In some aspects, deuterium is 80, 85, 90, 95, or 99% enriched at a desired location. Unless otherwise stated, enrichment at any point is above natural abundance and, in an aspect, is enough to alter a detectable property of the compounds as a drug in a human.

The compounds of the present disclosure may form a solvate with solvents (including water). Therefore, in one aspect, the disclosure includes a solvated form of the active compound. The term “solvate” refers to a molecular complex of a compound of the present disclosure (including a salt thereof) with one or more solvent molecules. Non-limiting examples of solvents are water, ethanol, dimethyl sulfoxide, acetone, and other common organic solvents. The term “hydrate” refers to a molecular complex comprising a disclosed compound and water. Pharmaceutically acceptable solvates in accordance with the disclosure include those wherein the solvent of crystallization may be isotopically substituted, e.g., D₂O, d₆-acetone, or d₆-DMSO. A solvate can be in a liquid or solid form.

It is known that chemical substances may form solids present in different states of order term polymorphic forms or modifications. The different forms of a polymorphic substance can differ greatly in their physical properties. The compounds disclosed herein can be present in different polymorphic forms, with it possible for particular forms to be metastable. Unless stated to the contrary, the present disclosure includes all such polymorphic forms.

### Proteolysis Targeting Chimeras (PROTACs)

The present disclosure provides proteolysis targeting chimeras (PROTACs) only capable of degradation of target proteins when exposed to the concurrent present of at least two stimuli. The presently disclosed PROTACs only become active under conditions in vivo where both stimuli are present (e.g., in pathological cells). The presently disclosed PROTACs would otherwise remain inactive with respect to degradation of target proteins under conditions where only one or no stimulus is present (e.g., in healthy cells). The PROTACs disclosed herein provide advantages over previously described PROTACS in terms of limiting off-target activity in healthy cells, as while one of the two stimuli might be present with some activity at another off-target location, the likelihood that two or more different stimuli would be concurrently present at the same off-target location becomes substantially lower.

In one aspect, a proteolysis targeting chimera (PROTAC) is provided of Formula I

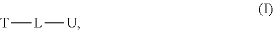

or a pharmaceutically acceptable salt or derivative thereof.

In some aspects of Formula I, T includes a target binding moiety. In some aspects, the target binding moiety is capable of binding to a target protein.

In some aspects of Formula I, L includes a linker moiety.

In some aspects of Formula I, U includes a ubiquitin ligase-recruiting moiety. In some aspects, the ubiquitin ligase-recruiting moiety is capable of binding a ubiquitin ligase.

In some aspects of Formula I, any of T, L, or U can further include a first stimulus reactive moiety Si. In some aspects, the first stimulus reactive moiety is reactive to a first stimulus. In some aspects of Formula I, T further includes the first stimulus reactive moiety. In some aspects of Formula I, L further includes the first stimulus reactive moiety. In some aspects of Formula I, U further includes the first stimulus reactive moiety.

In some aspects of Formula I, any of T, L, or U can further include a second stimulus reactive moiety S2. In some aspects, the second stimulus reactive moiety is reactive to a second stimulus. In some aspects, the second stimulus is different than the first stimulus. In some aspects of Formula I, T further includes the second stimulus reactive moiety. In some aspects of Formula I, L further includes the second stimulus reactive moiety. In some aspects of Formula I, U further includes the second stimulus reactive moiety.

In some aspects, upon exposure to only the first stimulus, only the second stimulus, or to neither of the first stimulus or second stimulus, the PROTAC of Formula I does not substantially recruit the target protein for ubiquitination and degradation by a proteasome. Upon exposure to only the first stimulus, the PROTAC of Formula I may form a reaction product resulting from a reaction of the first stimulus reactive moiety (via the first stimulus), where the reaction product does not substantially recruit the target protein for ubiquitination and degradation by a proteasome. Upon exposure to only the second stimulus, the PROTAC of Formula I may form a reaction product resulting from a reaction of the second stimulus reactive moiety (via the second stimulus), where the reaction product does not substantially recruit the target protein for ubiquitination and degradation by a proteasome.

In some aspects, upon exposure to both the first stimulus and the second stimulus, the PROTAC of Formula I forms an active compound capable of recruiting the target protein for ubiquitination and degradation by a proteasome. In such aspects, the active compound comprises a reaction product resulting from both a reaction of the first stimulus reactive moiety (via the first stimulus) and a reaction of the second stimulus reactive moiety (via the second stimulus). In some aspects, the PROTAC of Formula I only forms an active compound capable of recruiting the target protein for ubiquitination and degradation by a proteasome. Exposure to both the first stimulus and the second stimulus may occur either simultaneously or may alternatively occur sequentially (i.e., via a first exposure to the first stimulus followed by a second exposure to the second stimulus, or via a first exposure to the second stimulus followed by a second exposure to the first stimulus).

### Stimulus Reactive Moieties and Stimuli

The PROTACS described herein can include two or more stimulus reactive moieties as described herein. A stimulus reactive moiety refers to a group which, upon exposure to a stimulus, undergoes a chemical reaction. In some aspects, exposure of the stimulus reactive moiety to the stimulus results in degradation or alteration of the stimulus reactive moiety in the PROTAC. In some aspects, exposure of the stimulus reactive moiety to the stimulus may result in degradation of the stimulus reactive moiety (e.g., the breaking of covalent bonds between the stimulus reactive moiety and the remainder of the PROTAC), leading to exposure of a further functionality or moiety in the PROTAC which may have been directly attached (e.g., covalently linked) to the stimulus reactive moiety. In some aspects, exposure of the stimulus reactive moiety to the stimulus may result in alteration (e.g., chemical alteration) of the stimulus reactive moiety into a different moiety.

In some aspects, the PROTACs described herein include a first stimulus reactive moiety S¹ reactive to a first stimulus and a second stimulus reactive moiety S² reactive to a second stimulus. In some aspects, the first stimulus and the second stimulus are different.

In some aspects, the first stimulus reactive moiety and the second stimulus reactive moiety are not directly covalently bound to each other, i.e., while the first stimulus reactive moiety and the second stimulus reactive moiety can be both bound to the PROTACs described herein in various positions, the first stimulus reactive moiety and the second stimulus reactive moiety are never directly connected by only a covalent bond.

In some aspects, the first stimulus can include a stimulus present within a cell. In some aspects, the first stimulus can include a stimulus delivered exogenously to a cell.

In some aspects, the first stimulus can include hypoxia. In some aspects, the first stimulus reactive moiety can be reactive under conditions of hypoxia in a cell.

In some aspects, the first stimulus can include the presence or the elevation in the level of one or more reactive oxygen species (e.g., hydroxyl radical, superoxide, hydrogen peroxide, peroxynitrite, or singlet oxygen). In some aspects, the first stimulus reactive moiety can be reactive under conditions including the presence or elevation of the level of reactive oxygen species within a cell.

In some aspects, the first stimulus can include the presence or the elevation in the level of glutathione. In some aspects, the first stimulus reactive moiety can be reactive under conditions including the presence or the elevation in the level of glutathione within a cell.

In some aspects, the first stimulus can include the presence or the elevation in the level of one or more enzymes. In some aspects, the first stimulus reactive moiety can be reactive under conditions including the presence or the elevation in the level of one or more enzymes within a cell. In some aspects, the one or more enzymes can include a protease (e.g., a cathepsin such as cathepsin L or cathepsin B). In some aspects, the one or more enzymes can include penicillin G acylase (PGA). In some aspects, the one or more enzymes include NAD(P)H quinone dehydrogenase 1 (NQO1). In some aspects, the one or more enzymes include nitroreductase (NTR). In some aspects, the one or more enzymes include a histone deacetylase (HDAC).

In some aspects, the first stimulus can include exposure to light (e.g., visible or ultraviolet light or x-rays). In some aspects, the first stimulus reactive moiety can be reactive under conditions including exposure to light.

In some aspects, the first stimulus can include exposure to radiation. In some aspects, the first stimulus reactive moiety can be reactive under conditions including exposure to radiation.

Representative examples of first stimulus reactive moieties can include, but are not

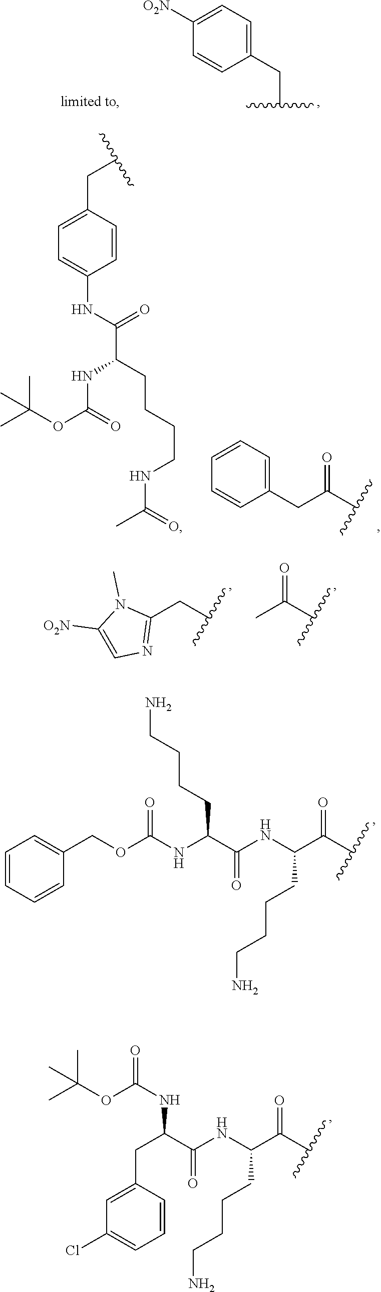

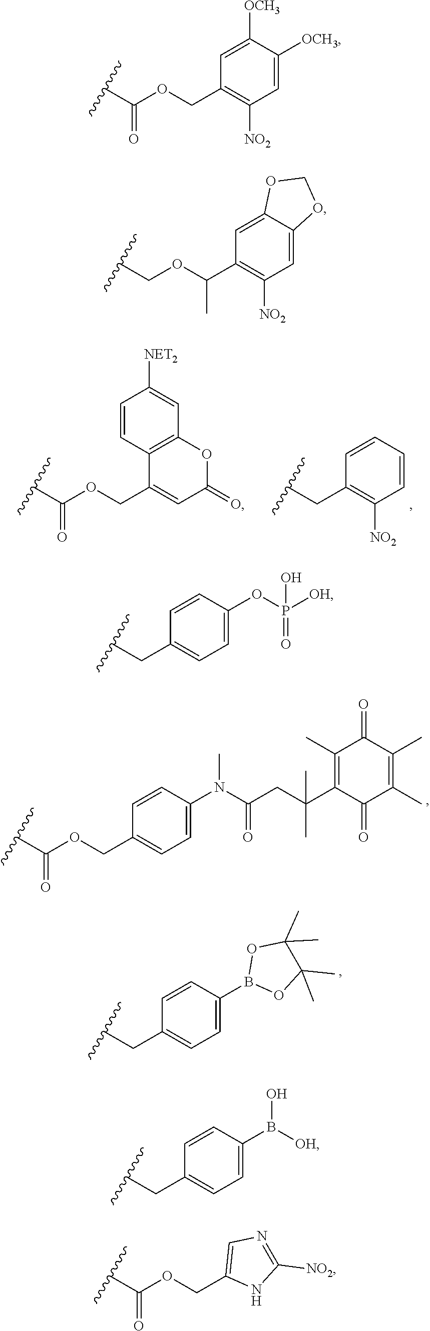

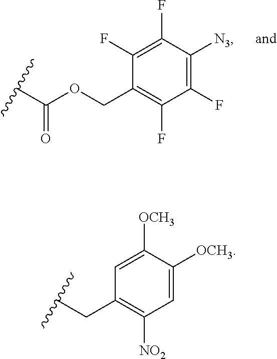 [SMILES: *C(=O)OCc1c(F)c(F)c(N=[N+]=[N-])c(F)c1F ; "and" ; *Cc1cc(OC)c(OC)cc1[N+](=O)[O-]]

In some aspects, the second stimulus can include a stimulus present within a cell. In some aspects, the second stimulus can include a stimulus delivered exogenously to a cell.

In some aspects, the second stimulus can include hypoxia. In some aspects, the second stimulus reactive moiety can be reactive under conditions of hypoxia in a cell.

In some aspects, the second stimulus can include the presence or the elevation in the level of one or more reactive oxygen species (e.g., hydroxyl radical, superoxide, hydrogen peroxide, peroxynitrite, or singlet oxygen). In some aspects, the second stimulus reactive moiety can be reactive under conditions including the presence or elevation of the level of reactive oxygen species within a cell.

In some aspects, the second stimulus can include the presence or the elevation in the level of glutathione. In some aspects, the second stimulus reactive moiety can be reactive under conditions including the presence or the elevation in the level of glutathione within a cell.

In some aspects, the second stimulus can include the presence or the elevation in the level of one or more enzymes. In some aspects, the second stimulus reactive moiety can be reactive under conditions including the presence or the elevation in the level of one or more enzymes within a cell. In some aspects, the one or more enzymes can include a protease (e.g., a cathepsin such as cathepsin L or cathepsin B). In some aspects, the one or more enzymes can include penicillin G acylase (PGA). In some aspects, the one or more enzymes include NAD(P)H quinone dehydrogenase 1 (NQO1). In some aspects, the one or more enzymes include nitroreductase (NTR). In some aspects, the one or more enzymes include a histone deacetylase (HDAC).

In some aspects, the second stimulus can include exposure to light (e.g., visible or ultraviolet light or x-rays). In some aspects, the second stimulus reactive moiety can be reactive under conditions including exposure to light.

In some aspects, the second stimulus can include exposure to radiation. In some aspects, the second stimulus reactive moiety can be reactive under conditions including exposure to radiation.

Representative examples of second stimulus reactive moieties can include, but are not limited to,

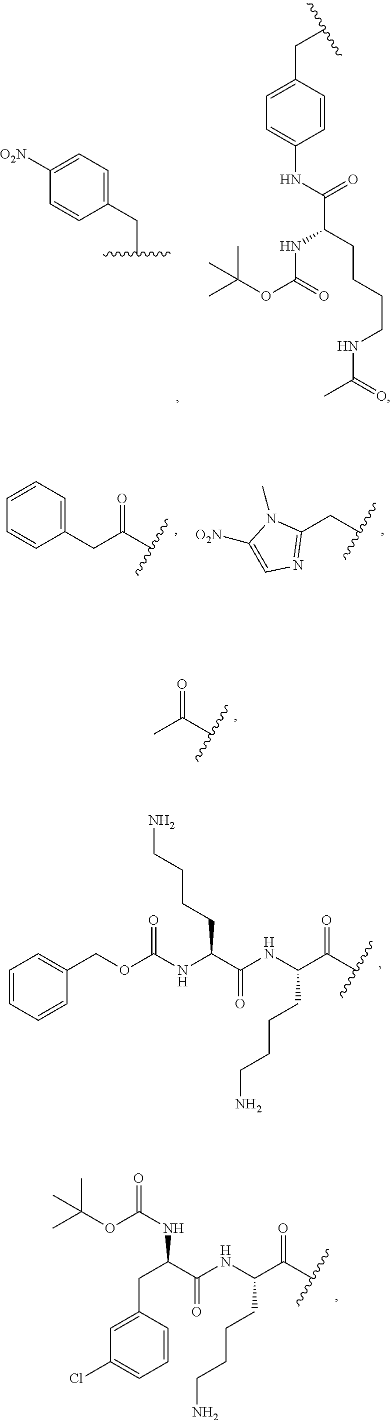

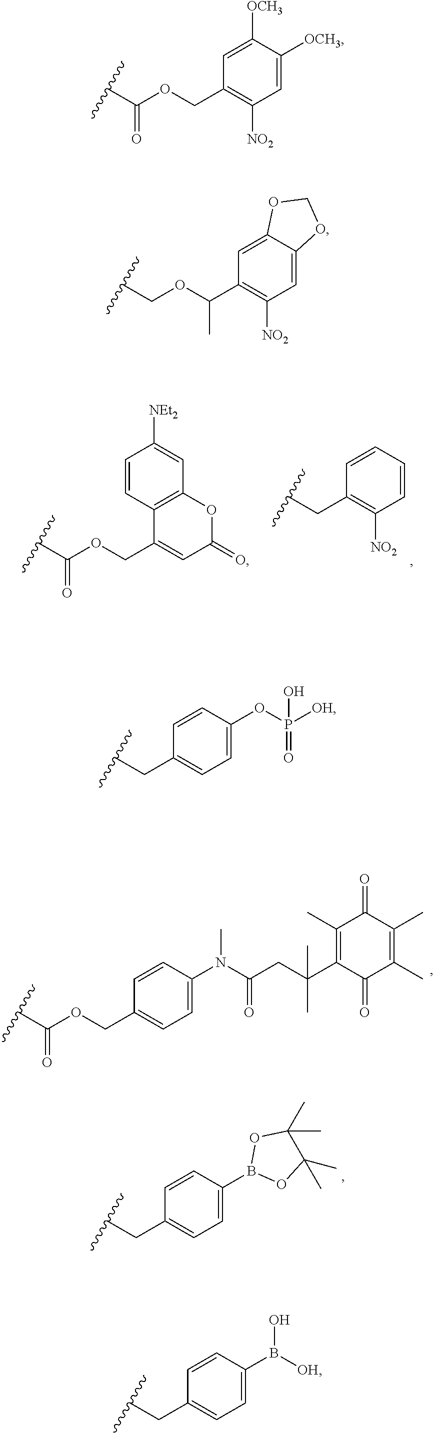

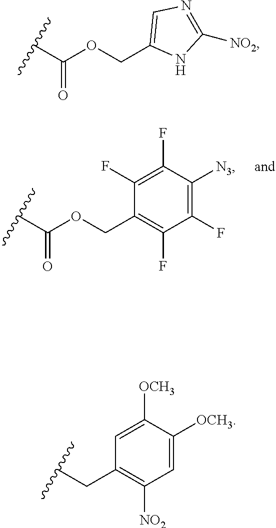 [SMILES: *C(=O)OCc1cnc([N+](=O)[O-])[nH]1 ; *C(=O)OCc1c(F)c(F)c(N=[N+]=[N-])c(F)c1F ; *Cc1cc(OC)c(OC)cc1[N+](=O)[O-]]

### Target Binding Moieties and Target Proteins

The PROTACs described herein further comprise one or more target-binding moieties. The target binding moiety is capable of binding to a target protein.

A “target protein” is used herein to describe a protein or polypeptide that is the target for binding to the target binding moiety according to the present disclosure. Target proteins may include any protein or peptide that may be bound by the target binding moiety, including fragments thereof, analogs thereof, and/or homologs thereof. Target proteins include proteins or peptides having any biological functional or activity, including structural, regulatory, hormonal, enzymatic, genetic, immunological, contractile, storage, transportation, and signal transduction. The target protein may include, in some aspects, structural proteins, receptors, enzymes, cell surface proteins, proteins pertinent to the integrated function of a cell, including proteins involved in catalytic activity, aromatase activity, motor activity, helicase activity, metabolic processes (anabolism and catabolism), antioxidant activity, proteolysis, biosynthesis, proteins with kinase activity, oxidoreductase activity, transferase activity, hydrolase activity, lyase activity, isomerase activity, ligase activity, enzyme regulatory activity, signal transducer activity, structural molecule activity, binding activity (for protein, lipid, or carbohydrate), receptor activity, cell motility, membrane fusion, cell communication, regulation of biological processes, development, cell differentiation, response to stimulus, behavioral proteins, cell adhesion proteins, proteins involved in cell death, proteins involved in transport including protein transporter activity, nuclear transport, iron transporter activity, channel transporter activity, carrier activity, permease activity, secretion activity, electron transporter activity, pathogenesis, chaperone regulator activity, nucleic acid binding activity, transcription regulator activity, extracellular organization and biogenesis activity, or translation regulator activity. Target proteins can include proteins from eukaryotes and prokaryotes, including microbes, viruses, fungi and parasites, including humans, microbes, viruses, fungi and parasites, among numerous others, including other animals, including domesticated animals, microbes, plants, and viruses.

The target protein may be endogenous or non-endogenous to the cell. In some aspects, the target protein is an endogenous protein. In some aspects, the target protein is an endogenous protein that mediates a disorder. The endogenous protein can be the normal form of the protein or an aberrant form. In some aspects, the target protein may be a mutant form of an endogenous protein associated with a specific disorder or condition, for example, a cancer, which may be, for example, a partial or full gain-of-function or loss-of-function mutant encoded by nucleotide polymorphisms. In some aspects, the modified ligand specifically targets an aberrant form of the target protein and not a normal form.

In some aspects, the target protein can be a non-endogenous protein, such as from a pathogen or toxin. In some aspects, the target protein can be a non-endogenous protein from a virus, for example, HIV, HBV, HCV, RSV, HPV, CMV, flavivirus, pestivirus, coronavirus, norovirus, etc. In some aspects, the target protein can be a non-endogenous protein from a bacteria, for example, a gram-positive or gram-negative bacteria, or mycobacteria. In some aspects, the target protein can be a non-endogenous protein from a fungus. In some aspects, the target protein can be a non-endogenous protein from a prion. In some aspects, the target protein can be a non-endogenous protein derived from a eukaryotic pathogen, such as a protist, helminth, etc.

Representative examples of target proteins include, but are not limited to, retinoid X receptor (RXR), dihydrofolate reductase (DHFR), heat shock protein 90 (HSP90), tyrosine kinase, aurora kinase, ATM, ATR, BPTF, ALK, ABL, JAK2, MET, mTORC1, mTORC2, Mast/stem cell growth factor receptor (SCFR), IGF1R, HDM2, MDM2, HDAC, RAF receptor, androgen receptor, estrogen receptor, thyroid hormone receptor, HIV protease, HIV integrase, AP1, AP2, MCL-1, DNA-PK, elF4E, IDH1, RAS, RASK, MERTK, MER, EGFR, FLT3, SMARCA2, CDK9, CDK12, CDK13, glucocorticoid receptor, RasG12C, Her3, Bcl-2, Bel-XL, PPAR-gamma, BCR-ABL, BRAF, LRRK2, PDGFRα, RET, fatty acid binding protein, FLAP, Kringle Domain V 4BVV, lactoylglutathione lyase, mPGES-1, Factor Xa, Kallikrein 7, Cathepsin K, Cathepsin L, Cathepsin S, MTH1, MDM4, PARP1, PARP2, PARP3, PARP14, PARP15, PDZ domain, phospholipase A2 domain, protein S100-A7 2WOS, NRASQ61K, NRASQ61R, TEAD1, TEAD2, TEAD3, TEAD4, Saposin-B, Sec7, pp60 SrC, Tanki, Ubc9 SUMO E2 ligase SF6D, Src, Src-AS1, Src-AS2, JAK3, MEK1, KIT, KSR1, CTNNB1, BCL6, PAK1, PAK4, TNIK, MEN1, ERK1, IDO1, CBP, ASH1L, ATAD2, YAP, BAZ2A, BAZ2B, BDRT, BDR9, SMARCA4, PB1, TRIM24, TIF1a, BRPF1, CECR2, CREBBP, PCAF, PHIP, TAF1, HDAC2, HDAC4, HDAC6, HDAC7, HDAC8, KAT2B, WWTR1, A2aR, alpha-subunit of FTase and/or GGTase, ARG1, B-TrCP, CBX7, Cdc7/ASK, Cdc7-Dbf4, KAT2A, HAT1, ATF2, KAT5, KDM1A, DOT1L, EHMT1, ceacam-1, CENP-E, clAP1/2, DKC1, DMT3A, DNA replication/repair protein, DNA2, DNMT3B, E2F1, EFHD2/SWIPROSIN, Eg5, EMI1, ERCCD1/XPF, EWS-FLI, FoxA1, GATA3, FOXP1, GCN2, GNAQ, GNAii, SETD2, SETD5, SETD8, SETDB1, SMYD2, SMYD3, SUV4-20H1, ErbB2 receptor, ErbB4 receptor, VEGFR1 receptor, VEGFR2 receptor, VEGFR3 receptor, PDGFRβ receptor, Lyn receptor, Hck receptor, c-MET receptor, TrkB receptor, Axl receptor, YES receptor, HER2, PNET receptor, RCC receptor, RAMP receptor, SEGA receptor, PDGFR receptors, ErbB2 receptor, HK2, HSP70, IAPs, IQGAP1, LSF, MCT1, MCT4, MEF2B, MMP3, MMP14, MUC1, MyB, Myd88, FGFR1 receptor, FGFR2 receptor, FGFR3 receptor, FGFR4 receptor, PDGRF receptor, DDR1 receptor, PDGRa receptor, PDGRP receptor, CDK4 receptor, CDK6 receptor, Fms receptor, T3151 VEGFR receptor, FGFR receptor, Flt 3 receptor, Eph2A receptor, JAK1 receptor, FKBP12 receptor, mTOR receptor, CDK 8 receptor, CDF-1R receptor, MEK2 receptor, Brk receptor, PI3Ka receptor, GCN5 receptor, G9a, EHMT2, EZH2, EED, PRMT3, PRMT4, PRMT5, PRMT6, NR2F6, NSD1, P70S6K, PIN1, SERCA, SF3B1, Sirtuin 2, Skp2, SMAD3, SPOP, Tall, KDM1, KDM4, KDM5, KDM6, L3MBTL3, Menin, HDAC6, HDAC7, PTP1B, SHP2, TBK1, Trib2, TRIF, TS, XPO1, RASN, ARIF1B Scavenger mRNA-decapping enzyme DcpS, ALK, BTK, NTRK1, NTRK2, NTRK3, IDO, ERK2, ABL1, ABL2, ATK1, ATK2, BMX, CSK, EPHA3, EPHA4, EPHA7, EPHB4, FES, FYN, GSG2, ISNR, HBV, CBL-B, ERK, WDR5, NSP3, IRAK4, NRAS, ADAR, ASCL1, PAX8, TP63, SARM1, Ataxin-2, KSR2, CSCR4, HDAC10, NSD2, WHSC1, RIT1, WRN, BAP1, EPAS1, HIF2a, GRB2, KMT2D, MLL2, MLL4, MLLT1, ENL, NSD3, PPM1D, WIP1, SOS1, TBXT, Brachyury, USP7, BKV, JCV, CK1α, GSPT1, ERF3, IFZV, TAU, CYP17A1, SALL4, FAM38, CYP20A1, HTT, NRF2, NFE2L2, P300, PIK3CA, SARM1, SNCA, MAPT, TCPTP, STAT3, MyD88, PTP4A3, SF3B1, ARID1B, and ARID2.

In some aspects, the target protein can include a tyrosine kinase (e.g., AATK, ABL, ABL2, ALK, AXL, BLK, BMX, BTK, CSF1R, CSK, DDR1, DDR2, EGFR, EPHA1, EPHA2, EPHA3, EPHA4, EPHA5, EPHA6, EPHA7, EPHA8, EPHA10, EPHB1, EPHB2, EPHB3, EPHB4, EPHB6, ERBB2, ERBB3, ERBB4, FER, FES<FGFR1, FGFR2, FGFR3, FGFR4, JAK1, JAK2, JAK3, KDR, KIT, KSR1, LCK1, LMTK2, LMTK3, LTK, LYN, MATK, MERTK, MET, MLTK, MST1R, MUSK, NKR1, NTRK1, NTRK2, NTRK3, PDGFRA, PDGFRB, PLK4, PTK2, PTK2B, PTK6, PTK7, RET, ROR1, ROR2, ROS1, RYK, SGK493, SRC, SRMS, STYK1, SYK, TEC, TEK, TEX14, TIE1, TNK1, TINK2, TNNI3K, TXK, TYK2, TYRO3, YES1, or ZAP70).

In some aspects, the target protein can include a serine/threonine kinase (e.g., casein kinase 2, protein kinase A, protein kinase B, protein kinase C, Raf kinases, CaM kinases, AKT1, AKT2, AKT3, ALK1, ALK2, ALK3, ALK4, Aurora A, Aurora B, Aurora C, CHK1, CHK2, CLK1, CLk2, CLK3, DAPK1, DAP2, DAPK3, DMPK, ERK1, ERK2, ERK5, GCK, GSK3, HIPK, KHS1, LKB1, LOK, MAPKAPK2, MAPKAPK, MNK1, MSSK1, MST1, MST2, MST4, NDR, NEK2, NEK3, NEK6, NEK7, NEK9, NEK11, PAK1, PAK2, PAK3, PAK4, PAK5, PAK6, PIM1, PIM2, PLK1, RIP2, RIPS, RSK1, RSK2, SGK2, SGK3, SIK1, STK33, TA01, TA02, TGF-beta, TLK2, TSSK1, TSSK2, ULK1, or ULK2).

In some aspects, the target protein can include a cyclin dependent kinase, for example, CDK1, CDK2, CDK3, CDK4, CDK5, CDK6, CDK7, CDK8, CDK9, CDK10, CDK11, CDK12, or CDK13.

In some aspects, the target protein can include a leucine-rich repeat kinase (e.g., LRRK2).

In some aspects, the target protein target can include a lipid kinase (e.g., PIK3CA, PIK3CB) or a sphingosine kinase (e.g., S1P).

In some aspects, the target protein can include a nuclear protein, for example BRD2, BRD3, BRD4, antennapedia homeodomain protein, BRCA1, BRCA2, CCAAT-Enhanced-Binding proteins, histones, polycomb-group proteins, high mobility group proteins, telomere binding proteins, FANCA, FANCD2, FANCE, FANCF, hepatocyte nuclear factors, Mad2, NF-kappa B, nuclear receptor coactivators, CREB-binding protein, p55, p107, p130, Rb proteins, p53, c-fos, c-jun, c-mdm2, c-myc, and c-rel.

The target binding moiety can include or be derived from any moiety identified as capable of binding to the target protein. In some aspects, the target binding moiety can be derived from a therapeutic agent capable of binding to the target protein.

The term “therapeutic agent” as used herein includes any synthetic or naturally occurring biologically active compound or composition of matter which, when administered to an organism (either human or a nonhuman animal), induces a desired pharmacologic, immunogenic, and/or physiologic effect by local and/or systemic action. The term therefore encompasses those compounds or chemicals traditionally regard as drugs, vaccines, and biopharmaceuticals including molecules such as proteins, peptides, hormones, nucleic acids, gene constructs and the like. Examples of therapeutic agents are described in well-known literature references such as the Merk Index (14^{th} Edition), the Physician's Desk Reference (64^{th} Edition), and The Pharmacological Basis of Therapeutics (12^{th} Edition), and they include, without limitation, medicaments; vitamins; mineral supplements, substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiandrenergics, antiarrhythmics, antihypertensive agents, hormones, and nutrients, antiarthritics, antiasthmatic agents, anticonvulsants, antihistamines, antinauseants, antineoplastics, antipruritics, antipyretics, antispasmodics, cardiovascular preparations (including calcium channel blockers, beta blockers, and beta-agonists), antihypertensives, diuretics, vasodilators, central nervous system stimulants, cough and cold preparations, decongestants, diagnostics, bone growth stimulants and bone resorption inhibitors, immunosuppressives, muscle relaxants, psychostimulants, sedatives, tranquilizers, proteins, peptides, and fragments thereof (whether naturally occurring, chemically synthesized or recombinantly produced), and nucleic acid molecules (polymeric forms of two or more nucleotides, either ribonucleotides (RNA) or deoxyribonucleotides (DNA) including both double and single-stranded molecules, gene constructs, expression vectors, antisense molecules and the like), small molecules and other biologically active macromolecules such as, for examples, proteins and enzymes. The agent may be a biologically active agent used in medical, including veterinary applications, and in agriculture, such as with plants, as well as other areas.

In some aspects, the target binding moiety can include or be derived from a ligand for retinoid X receptor (RXR), dihydrofolate reductase (DHFR), heat shock protein 90 (HSP90), tyrosine kinase, aurora kinase, ATM, ATR, BPTF, ALK, ABL, JAK2, MET, mTORC1, mTORC2, Mast/stem cell growth factor receptor (SCFR), IGF1R, HDM2, MDM2, HDAC, RAF receptor, androgen receptor, estrogen receptor, thyroid hormone receptor, HIV protease, HIV integrase, AP1, AP2, MCL-1, DNA-PK, elF4E, IDH1, RAS, RASK, MERTK, MER, EGFR, FLT3, SMARCA2, CDK9, CDK12, CDK13, glucocorticoid receptor, RasG12C, Her3, Bcl-2, Bel-XL, PPAR-gamma, BCR-ABL, BRAF, LRRK2, PDGFRα, RET, fatty acid binding protein, FLAP, Kringle Domain V 4BVV, lactoylglutathione lyase, mPGES-1, Factor Xa, Kallikrein 7, Cathepsin K, Cathepsin L, Cathepsin S, MTH1, MDM4, PARP1, PARP2, PARP3, PARP14, PARP15, PDZ domain, phospholipase A2 domain, protein S100-A7 2WOS, NRASQ61K, NRASQ61R, TEAD1, TEAD2, TEAD3, TEAD4, Saposin-B, Sec7, pp60 SrC, Tanki, Ubc9 SUMO E2 ligase SF6D, Src, Src-AS1, Src-AS2, JAK3, MEK1, KIT, KSR1, CTNNB1, BCL6, PAK1, PAK4, TNIK, MEN1, ERK1, IDO1, CBP, ASH1L, ATAD2, YAP, BAZ2A, BAZ2B, BDRT, BDR9, SMARCA4, PB1, TRIM24, TIF1a, BRPF1, CECR2, CREBBP, PCAF, PHIP, TAF1, HDAC2, HDAC4, HDAC6, HDAC7, HDAC8, KAT2B, WWTR1, A2aR, alpha-subunit of FTase and/or GGTase, ARG1, B-TrCP, CBX7, Cdc7/ASK, Cdc7-Dbf4, KAT2A, HAT1, ATF2, KAT5, KDM1A, DOT1L, EHMT1, ceacam-1, CENP-E, clAP1/2, DKC1, DMT3A, DNA replication/repair protein, DNA2, DNMT3B, E2F1, EFHD2/SWIPROSIN, Eg5, EMI1, ERCCD1/XPF, EWS-FLI, FoxA1, GATA3, FOXP1, GCN2, GNAQ, GNAii, SETD2, SETD5, SETD8, SETDB1, SMYD2, SMYD3, SUV4-20H1, ErbB2 receptor, ErbB4 receptor, VEGFR1 receptor, VEGFR2 receptor, VEGFR3 receptor, PDGFRβ receptor, Lyn receptor, Hck receptor, c-MET receptor, TrkB receptor, Axl receptor, YES receptor, HER2, PNET receptor, RCC receptor, RAMP receptor, SEGA receptor, PDGFR receptors, ErbB2 receptor, HK2, HSP70, IAPs, IQGAP1, LSF, MCT1, MCT4, MEF2B, MMP3, MMP14, MUC1, MyB, Myd88, FGFR1 receptor, FGFR2 receptor, FGFR3 receptor, FGFR4 receptor, PDGRF receptor, DDR1 receptor, PDGRα receptor, PDGRβ receptor, CDK4 receptor, CDK6 receptor, Fms receptor, T3151 VEGFR receptor, FGFR receptor, Flt 3 receptor, Eph2A receptor, JAK1 receptor, FKBP12 receptor, mTOR receptor, CDK 8 receptor, CDF-1R receptor, MEK2 receptor, Brk receptor, PI3Ka receptor, GCN5 receptor, G9a, EHMT2, EZH2, EED, PRMT3, PRMT4, PRMT5, PRMT6, NR2F6, NSD1, P70S6K, PIN1, SERCA, SF3B1, Sirtuin 2, Skp2, SMAD3, SPOP, Tali, KDM1, KDM4, KDM5, KDM6, L3MBTL3, Menin, HDAC6, HDAC7, PTP1B, SHP2, TBK1, Trib2, TRIF, TS, XPO1, RASN, ARIF1B Scavenger mRNA-decapping enzyme DcpS, ALK, BTK, NTRK1, NTRK2, NTRK3, IDO, ERK2, ABL1, ABL2, ATK1, ATK2, BMX, CSK, EPHA3, EPHA4, EPHA7, EPHB4, FES, FYN, GSG2, ISNR, HBV, CBL-B, ERK, WDR5, NSP3, IRAK4, NRAS, ADAR, ASCL1, PAX8, TP63, SARM1, Ataxin-2, KSR2, CSCR4, HDAC10, NSD2, WHSC1, RIT1, WRN, BAP1, EPAS1, HIF2a, GRB2, KMT2D, MLL2, MLL4, MLLT1, ENL, NSD3, PPM1D, WIP1, SOS1, TBXT, Brachyury, USP7, BKV, JCV, CK1α, GSPT1, ERF3, IFZV, TAU, CYP17A1, SALL4, FAM38, CYP20A1, HTT, NRF2, NFE2L2, P300, PIK3CA, SARM1, SNCA, MAPT, TCPTP, STAT3, MyD88, PTP4A3, SF3B1, ARID1B, or ARID2.

In some aspects, the target binding moiety can include or be derived from a ligand for a tyrosine kinase (e.g., AATK, ABL, ABL2, ALK, AXL, BLK, BMX, BTK, CSF1R, CSK, DDR1, DDR2, EGFR, EPHA1, EPHA2, EPHA3, EPHA4, EPHA5, EPHA6, EPHA7, EPHA8, EPHA10, EPHB1, EPHB2, EPHB3, EPHB4, EPHB6, ERBB2, ERBB3, ERBB4, FER, FES<FGFR1, FGFR2, FGFR3, FGFR4, JAK1, JAK2, JAK3, KDR, KIT, KSR1, LCK1, LMTK2, LMTK3, LTK, LYN, MATK, MERTK, MET, MLTK, MST1R, MUSK, NKR1, NTRK1, NTRK2, NTRK3, PDGFRA, PDGFRB, PLK4, PTK2, PTK2B, PTK6, PTK7, RET, ROR1, ROR2, ROS1, RYK, SGK493, SRC, SRMS, STYK1, SYK, TEC, TEK, TEX14, TIE1, TNK1, TINK2, TNNI3K, TXK, TYK2, TYRO3, YES1, or ZAP70).

In some aspects, the target binding moiety can include or be derived from a ligand for a serine/threonine kinase (e.g., casein kinase 2, protein kinase A, protein kinase B, protein kinase C, Raf kinases, CaM kinases, AKT1, AKT2, AKT3, ALK1, ALK2, ALK3, ALK4, Aurora A, Aurora B, Aurora C, CHK1, CHK2, CLK1, CLk2, CLK3, DAPK1, DAP2, DAPK3, DMPK, ERK1, ERK2, ERK5, GCK, GSK3, HIPK, KHS1, LKB1, LOK, MAPKAPK2, MAPKAPK, MNK1, MSSK1, MST1, MST2, MST4, NDR, NEK2, NEK3, NEK6, NEK7, NEK9, NEK11, PAK1, PAK2, PAK3, PAK4, PAK5, PAK6, PIM1, PIM2, PLK1, RIP2, RIPS, RSK1, RSK2, SGK2, SGK3, SIK1, STK33, TA01, TA02, TGF-beta, TLK2, TSSK1, TSSK2, ULK1, or ULK2).

In some aspects, the target binding moiety can include or be derived from a ligand for a cyclin dependent kinase, for example, CDK1, CDK2, CDK3, CDK4, CDK5, CDK6, CDK7, CDK8, CDK9, CDK10, CDK11, CDK12, or CDK13.

In some aspects, the target binding moiety can include or be derived from a ligand for a leucine-rich repeat kinase (e.g., LRRK2).

In some aspects, the target binding moiety can include or be derived from a ligand for a lipid kinase (e.g., PIK3CA, PIK3CB) or a sphingosine kinase (e.g., S1P).

In some aspects, the target protein target binding moiety can include or be derived from a ligand for a nuclear protein, for example BRD2, BRD3, BRD4, antennapedia homeodomain protein, BRCA1, BRCA2, CCAAT-Enhanced-Binding proteins, histones, polycomb-group proteins, high mobility group proteins, telomere binding proteins, FANCA, FANCD2, FANCE, FANCF, hepatocyte nuclear factors, Mad2, NF-kappa B, nuclear receptor coactivators, CREB-binding protein, p55, p107, p130, Rb proteins, p53, c-fos, c-jun, c-mdm2, c-myc, and c-rel.

In some particular aspects, the target binding moiety can include or be derived from an anti-cancer agent. In some aspects, the target binding moiety can include or be derived from a chemotherapeutic agent, for example but not limited to, azacytidine, capecitabine, carmofur, cladribine, clofarabine, cytarabine, decitabine, floxuridine, fludarabine, fluorouracil, gemcitabine, mercaptopurine, melarabine, pentostatin, tegafur, tioguanine, methotrexate, pemetrexed, raltitrexed, hydroxycarbamide, irinotecan, topotecan, daunorubicin, doxorubicin, epirubicin, idarubicin, mitoxantrone, valrubicin, etoposide, teniposide, cabazitaxel, docetaxel, paclitaxel, vinblastine, vincristine, vindesine, vinflunine, vinorelbine, bendamustine, busulfan, carmustine, chlorambucil, chlormethine, cyclophosphamide, dacarbazine, fotemustine, ifosfamide, lomustine, melphalan, streptozotocin, temozolomide, carboplatin, cisplatin, nedaplatin, oxaliplatin, altretamine, bleomycin, bortezomib, dactinomycin, estramustine, ixabepilone, mitomycin, procarbazine, afatanib, aflibercept, axitinib, bosutinib, crizotinib, dasatinib, erlotinib, gefitinib, imatinib, lapatinib, nilotinib, pazopanib, ponatinib, regorafenib, ruxotinib, sorafenib, sunitinib, vandetanib, everolimus, temsirolimus, alitretinoin, bexarotene, isotretinoin, tamibarotene, tretinoin, lenalidomide, pomalidomide, thalidomide, Panobinostat, romidepsin, valproate, vorinostat, anagrelide, and vemurafenib. In some aspects, the target binding moiety may be derived from a targeted cancer therapy, for example imatinib, defitinib, erlotinib, sorafenib, sunitinib, dasatinib, lapatinib, nilotinib, bortezomib, tamoxifen, Janus kinase inhibitors (e.g., tofacitinib), ALK inhibitors (e.g., crizotinib), Bcl-2 inhibitors (e.g., venetoclax, obatoclax, navitoclax, and gossypol), PARP inhibitors, (e.g., olaparib, rucaparib, niraparib, and talazoparib), PI3K inhibitors (e.g., perifosine), apatanib, Braf inhibitors (e.g., vemurafenib, dabrafenib, LGX818), MEK inhibitors (e.g., trametinib, MEK162), CDK inhibitors (e.g., PD-0332991, LEEo11), Hsp90 inhibitors, hedgehog pathway inhibitors (e.g., vismodegib or sonidegib), salinomycin, temsirolimus, everolimus, vemurafenib, trametinib, and dabrafenib. Other anti-cancer therapeutics from which PBM may be derived include afatinib, brigatinib, dacomitinib, erlotinib, gefitinib, icotinib, mobocertinib, olmutinib, Osimertinib, rociletinib, vandetanib, lapatinib, neratinib, tucatinib, avapritinib, axitinib, masitinib, pazopanib, ripretinib, sorafenib, sunitinib, toceranib, lestaurtinib, gilteritinib, axitinib, cediranib, lenvatinib, nintedanib, pazopanib, regorafenib, semaxanib, sorafenib, sunitinib, tivozanib, toceranib, vandetanib, alectinib, brigatinib, ceritinib, entrectinib, larotrectinib, infigratinib, pemigatinib, pralsetinib, selpercatinib, vandetanib, cabozantinib, capmatinib, crizotinib, asciminib, bosutinib, dasatinib, imatinib, nilotinib, panotinib, radotinib, baracitinib, fedratinib, filgotinib, lestaurtinib, momelotinib, pacritinib, ruxolitinib, binimetinib, cobimetinib, selumetinib, trametinib, crizotinib, entrectinib, lorlatinib, acalaburitnib, ibrutinib, zanubrutinib, aflibercept, everolimus, ridaforolimus, temsirolimus, glasdegib, sonidegib, vismodegib, abemaciclib, palbociclib, ribociclib, trilaciclib, cabozantinib, capmatinib, entrectinib, erdafitinib, gilteritinib, larotrectinib, Lenvatinib, masitinib, midostaurin, nintedanib, pazopanib, pemigatinib, pexidartinib, quizartinib, regoragenib, ripretanib, sorafenib, sotorasib, sunitinib, tepotinib, vandetanib, and venetoclax.

### Ubiquitin Ligase-Recruiting Moieties

The PROTACs described herein can further include one or more ubiquitin ligase-recruiting moieties. The ubiquitin ligase-recruiting moiety is a chemical moiety capable of recruiting an ubiquitin ligase to a given target protein resulting in its targeted degradation. In some aspects, U is a chemical moiety based upon a high-affinity small molecule for E3 ubiquitin ligases, such as von Hippel-Lindau or cereblon. In some aspects, U is a chemical moiety based upon a von Hippel-Lindau binder such as VH032 or VH298. In some aspects, U is a chemical moiety based upon a cereblon binder such as thalidomide, lenalidomide, or pomalidomide.

In some aspects, the ubiquitin ligase-recruiting moiety is selected from

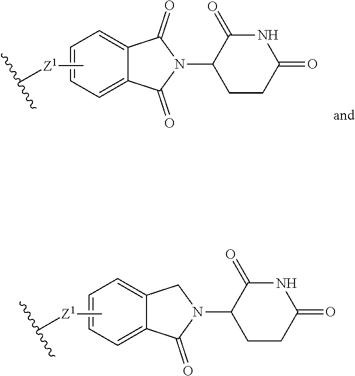

wherein Z¹ is selected from a bond, O, N(R^{Z}), and CH₂, and wherein R^{Z} is selected from hydrogen and C₁-C₆ alkyl.

In some aspects, the ubiquitin ligase-recruiting moiety is selected from

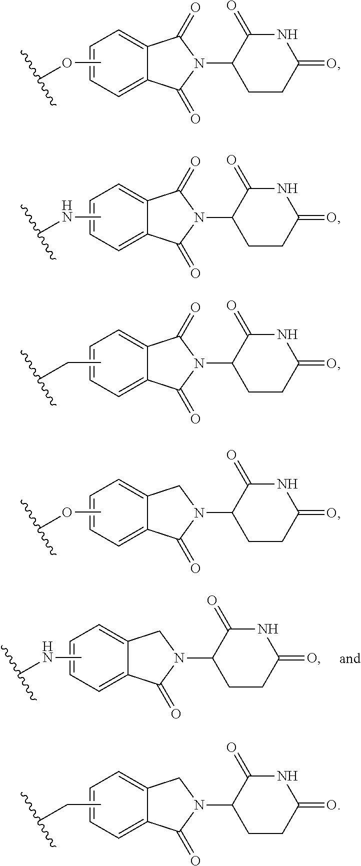

In some aspects of Formula I, the ubiquitin ligase-recruiting moiety is selected from

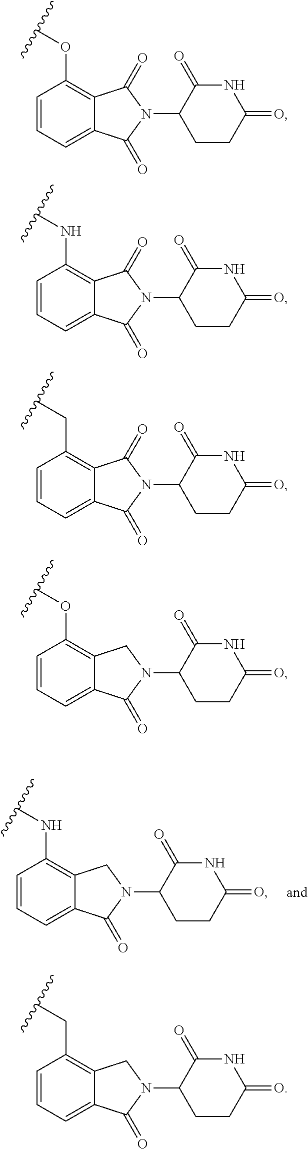

In some aspects of Formula I, the ubiquitin ligase-recruiting moiety is selected from

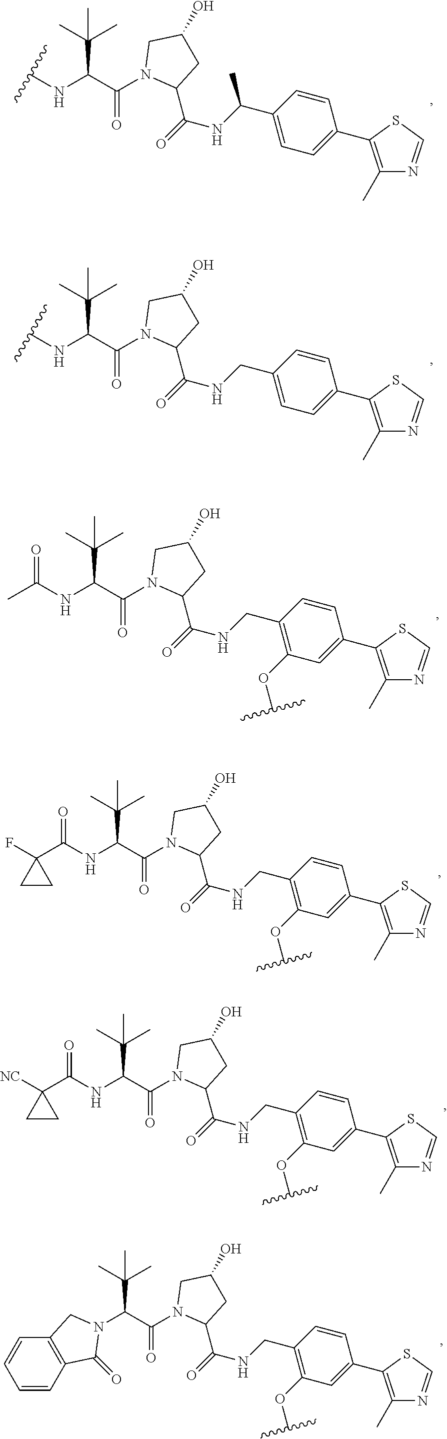 [SMILES: *N[C@H](C(=O)N1C[C@H](O)CC1C(=O)N[C@@H](C)c1ccc(-c2scnc2C)cc1)C(C)(C)C ; *N[C@H](C(=O)N1C[C@H](O)CC1C(=O)NCc1ccc(-c2scnc2C)cc1)C(C)(C)C ; *Oc1cc(-c2scnc2C)ccc1CNC(=O)C1C[C@@H](O)CN1C(=O)[C@@H](NC(C)=O)C(C)(C)C ; *Oc1cc(-c2scnc2C)ccc1CNC(=O)C1C[C@@H](O)CN1C(=O)[C@@H](NC(=O)C1(F)CC1)C(C)(C)C ; *Oc1cc(-c2scnc2C)ccc1CNC(=O)C1C[C@@H](O)CN1C(=O)[C@@H](NC(=O)C1(C#N)CC1)C(C)(C)C ; *Oc1cc(-c2scnc2C)ccc1CNC(=O)C1C[C@@H](O)CN1C(=O)[C@@H](N1Cc2ccccc2C1=O)C(C)(C)C]

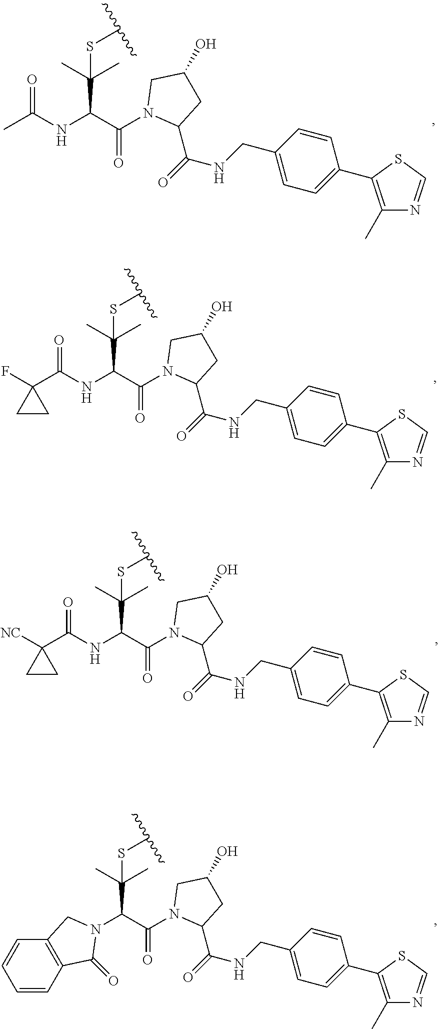 [SMILES: *SC(C)(C)[C@H](NC(C)=O)C(=O)N1C[C@H](O)CC1C(=O)NCc1ccc(-c2scnc2C)cc1 ; *SC(C)(C)[C@H](NC(=O)C1(F)CC1)C(=O)N1C[C@H](O)CC1C(=O)NCc1ccc(-c2scnc2C)cc1 ; *SC(C)(C)[C@H](NC(=O)C1(C#N)CC1)C(=O)N1C[C@H](O)CC1C(=O)NCc1ccc(-c2scnc2C)cc1 ; *SC(C)(C)[C@@H](C(=O)N1C[C@H](O)CC1C(=O)NCc1ccc(-c2scnc2C)cc1)N1Cc2ccccc2C1=O]

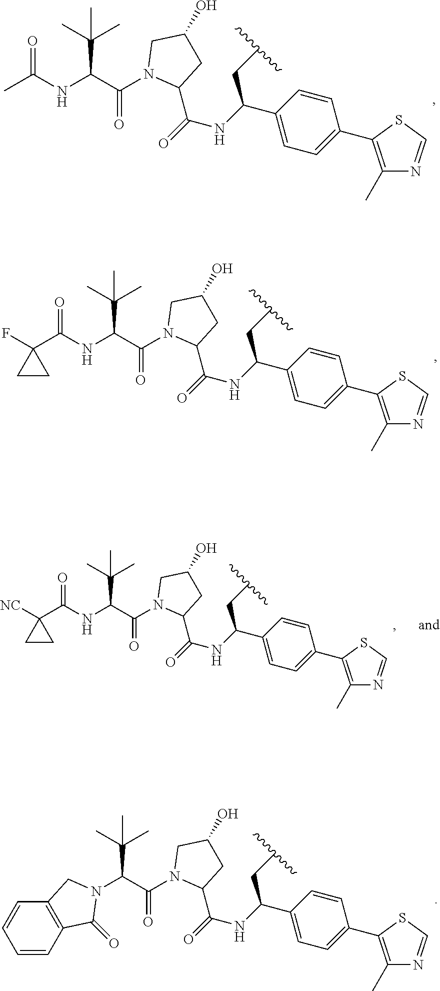 [SMILES: *C[C@H](NC(=O)C1C[C@@H](O)CN1C(=O)[C@@H](NC(C)=O)C(C)(C)C)c1ccc(-c2scnc2C)cc1 ; *C[C@H](NC(=O)C1C[C@@H](O)CN1C(=O)[C@@H](NC(=O)C1(F)CC1)C(C)(C)C)c1ccc(-c2scnc2C)cc1 ; *C[C@H](NC(=O)C1C[C@@H](O)CN1C(=O)[C@@H](NC(=O)C1(C#N)CC1)C(C)(C)C)c1ccc(-c2scnc2C)cc1 ; *C[C@H](NC(=O)C1C[C@@H](O)CN1C(=O)[C@@H](N1Cc2ccccc2C1=O)C(C)(C)C)c1ccc(-c2scnc2C)cc1]

In some aspects, the ubiquitin ligase-recruiting moiety is selected from:

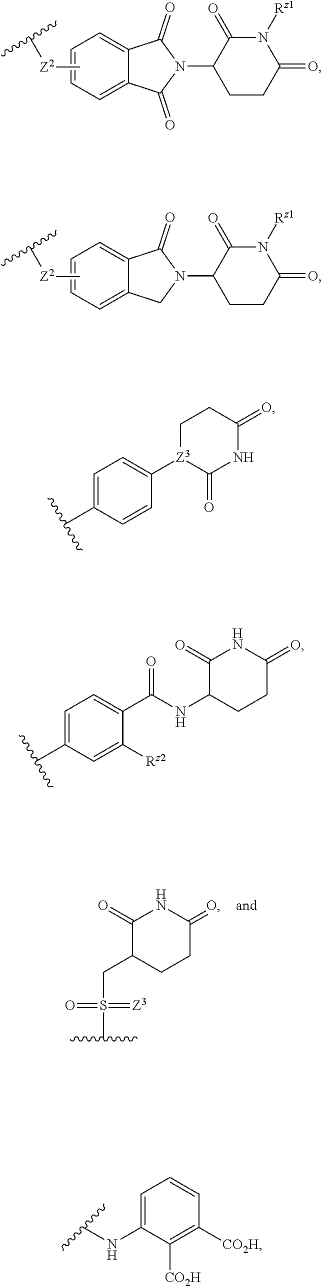

wherein:
Z² is a bond or selected from —NH—, —O—, and —CH₂—;
R^{Z1} is hydrogen or —CH₂—O—C(═O)—O—(C₁-C₆ alkyl);
Z³ is selected from CH and N;
R^{Z2} is selected from hydrogen, halogen, C₁-C₆ alkyl, C₁-C₆ haloalkyl, and C₁-C₆ alkoxy; and
Z³ is selected from O and NH.

### Linker Moieties

The PROTACs described herein can further include one or more linker moieties. A linker moiety is a chemically stable bivalent group that attaches T to U. A linker moiety as described herein can be used in either direction, i.e., either the left end is linked to T and the right end to U, or the left end is linked to U and the right end to T.

In some aspects, the linker moiety is a chain of 2 to 14, 15, 16, 17, 18, 19, or 20 or more carbon atoms, of which one or more carbons can be optionally replaced by a heteroatom such as O, N, S, or P as allowed by valency.

In some aspects, the chain has 2, 3, 4, 5, 6, 7, 8, 9, 10, 11, 12, 13, 14, 15, 16, 18, 19, 19, or 20 contiguous atoms. For example, the chain may include 1 or more ethylene glycol units that can be contiguous, partially contiguous, or non-contiguous (for example, 2, 3, 4, 5, 6, 7, 8, 9, 10, 11, or 12 ethylene glycol units).

In some aspects, the chain has at least 1, 2, 3, 4, 5, 6, 7, or 8 contiguous units which can be branched and which can be independently C₁-C₂₀ alkyl, 6- to 10-membered monocyclic or bicyclic aryl, 5- to 10-membered monocyclic or bicyclic heteroaryl, C₂-C₂₀ alkenyl, or C₂-C₂₀ alkynyl, C₃-C₇ cycloalkyl, or 3- to 8-membered monocyclic or bicyclic heterocycle substituents.

In some aspects, the linker moiety can include one or more ethylene glycol, propylene glycol, lactic and/or glycolic acid units. Block and random lactic acid-co-glycolic acid moieties, as well as ethylene glycol and propylene glycol, are known in the art and can be modified to obtain the desired half-life and hydrophilicity. In certain aspects, these units can be flanked or interspersed with other moieties, such as, for example, C₁-C₂₀ alkyl, 6- to 10-membered monocyclic or bicyclic aryl, 5- to 10-membered monocyclic or bicyclic heteroaryl, C₃-C₇ cycloalkyl, 3- to 8-membered monocyclic or bicyclic heterocycle, etc., each of which can be interspersed with optionally substituted O, N, S, P or Si atoms as allowed by valency, as desired to achieve the appropriate properties.

In some aspects, the linker moiety is an optionally substituted (poly)ethylene glycol having at least 1, at least 2, at least 3, at least 4, at least 5, at least 6, at least 7, at least 8, at least 9, at least 10, or more, ethylene glycol units, or optionally substituted C₁-C₂₀ alkyl groups interspersed with optionally substituted O, N, S, P or Si atoms as allowed by valency.

In some aspects, the linker moiety is flanked, substituted, or interspersed with a C₁-C₂₀ alkyl, C₃-C₇ cycloalkyl, 3- to 8-membered monocyclic or bicyclic heterocycle, 6- to 10-membered monocyclic or bicyclic aryl, or 5- to 10-membered monocyclic or bicyclic heteroaryl group.

In some aspects, the linker moiety may be asymmetric or symmetric.

In some aspects, the linker moiety can be a non-linear chain, and can be, or include, C₃-C₇ cycloalkyl, 3- to 8-membered monocyclic or bicyclic heterocycle, 6- to 10-membered monocyclic or bicyclic aryl, or 5- to 10-membered monocyclic or bicyclic heteroaryl moieties.

In some aspects, the linker moiety is selected from L1:

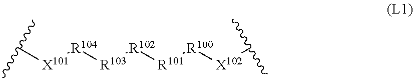

In some aspects, the linker moiety is selected from the group consisting of a moiety of Formula L1, Formula L2, Formula L3, Formula L4, Formula L5, Formula L6, Formula L7, Formula L8, Formula L9, or Formula Lio:

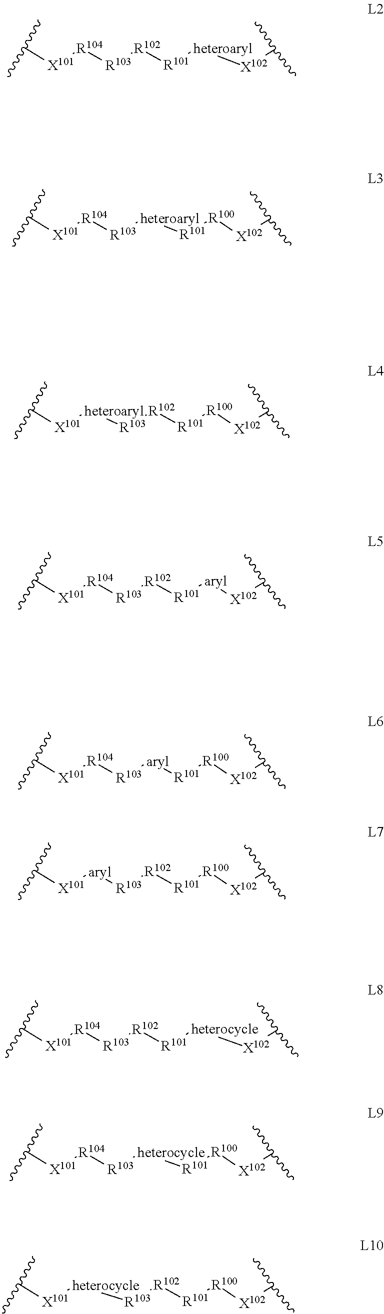

wherein:
X¹⁰¹ and X¹⁰² are independently at each occurrence selected from a bond, 6- to 10-membered monocyclic or bicyclic aryl, 5- to 10-membered monocyclic or bicyclic heteroaryl, C₃-C₇ cycloalkyl, 3- to 8-membered monocyclic or bicyclic heterocycle, C₁-C₂₀ alkyl, C₂-C₂₀ alkenyl, C₂-C₂₀ alkynyl, NR¹⁰⁵, O, C(O), and S;
R¹⁰⁰, R¹⁰¹, R¹⁰², R¹⁰³, and R¹⁰⁴ are independently at each occurrence selected from the group consisting of a bond, —C(O)—, —C(O)O—, —OC(O)—, —SO₂—, —S(O)—, C(S)—, —C(O)NR¹⁰⁵—, —NR¹⁰⁵C(O)—, —O—, —S—, —NR¹⁰⁵—, —P(O)(OR¹⁰⁵))—, C₁-C₂₀ alkyl, C₂-C₂₀ alkenyl, C₂-C₂₀ alkynyl, 6- to 10-membered monocyclic or bicyclic aryl, 3- to 8-membered monocyclic or bicyclic heterocycle, C₃-C₇ cycloalkyl, 5- to 10-membered monocyclic or bicyclic heteroaryl, lactic acid, or glycolic acid, each of which may be optionally substituted with one or more (for example, 1, 2, 3, or 4) substituents independently selected from R¹⁴⁰;
R¹⁰⁵ independently selected at each occurrence from R¹²⁰, R¹¹⁰C(O)—, R¹¹⁰C(N)—, R¹¹⁰S(O)—, and R¹¹⁰S(O)₂—, each of which may be optionally substituted with one or more R¹⁴⁰ groups as allowed by valency;
R¹¹⁰ is independently selected at each occurrence from hydrogen, halo, C₁-C₆alkyl, C₁-C₆haloalkyl, C₂-C₆alkenyl, C₂-C₆alkynyl, (C₃-C₇ cycloalkyl)-(C₀-C₆ alkyl)-, (4- to 6-membered heterocycle)-(C₀-C₆ alkyl)-, (5- to 10-membered monocyclic or bicyclic aryl)-(C₀-C₆ alkyl)-, (5- to 10-membered monocyclic or bicyclic heteroaryl)-(C₀-C₆ alkyl)-, —OR¹²⁰, —SR¹²⁰, and —NR¹²⁰R¹³⁰, each of which may be optionally substituted with one or more R¹⁴⁰ groups as allowed by valency; and
R¹²⁰ and R¹³⁰ are independently selected at each occurrence from hydrogen, C₁-C₆alkyl, C₁-C₆haloalkyl, C₂-C₆alkenyl, C₂-C₆alkynyl, (C₃-C₇ cycloalkyl)-(C₀-C₆ alkyl)-, (4- to 6-membered heterocycle)-(C₀-C₆ alkyl)-, (5- to 10-membered monocyclic or bicyclic aryl)-(C₀-C₆ alkyl)-, (5- to 10-membered monocyclic or bicyclic heteroaryl)-(C₀-C₆ alkyl)-, each of which may be optionally substituted by one or more R¹⁴⁰ groups as allowed by valency; and
R¹⁴⁰ is independently at each occurrence selected from the group consisting of hydrogen, halo, nitro, cyano, azido, oxo, C₁-C₆ alkyl, C₁-C₆ haloalkyl, C₂-C₆ alkenyl, C₂-C₆ alkynyl, (C₃-C₇ cycloalkyl)(C₀-C₆ alkyl)-, (3- to 8-membered monocyclic or bicyclic heterocycle)-(C₀-C₆ alkyl)-, (6- to 10-membered monocyclic or bicyclic aryl)-(C₀-C₆ alkyl)-, (5- to 10-membered monocyclic or bicyclic heteroaryl)-(C₀-C₆ alkyl)-, R¹⁴¹O—, R¹⁴¹S—, R¹⁴¹R¹⁴²N—, R¹⁴³C(O)—, R¹⁴³C(O)—O—, R¹⁴²C(O)—NR¹⁴²—, R¹⁴³S(O)₂—, R¹⁴³S(O)₂—O—, and R¹⁴³S(O)₂—NR¹⁴²—, wherein R¹⁴³ is independently selected at each occurrence from R¹⁴¹, halo, R¹⁴¹O—, and R¹⁴¹R¹⁴²N—, and wherein R¹⁴¹ and R¹⁴² are independently selected at each occurrence from hydrogen, C₁-C₆ alkyl, C₁-C₆ haloalkyl, C₂-C₆ alkenyl, C₂-C₆ alkynyl, (C₃-C₇ cycloalkyl)(C₀-C₆ alkyl)-, (3- to 8-membered monocyclic or bicyclic heterocycle)-(C₀-C₆ alkyl)-, (6- to 10-membered monocyclic or bicyclic aryl)-(C₀-C₆ alkyl)-, and (5- to 10-membered monocyclic or bicyclic heteroaryl)-(C₀-C₆ alkyl)-.

In some aspects, R¹⁰⁰, R¹⁰¹, R¹⁰², R¹⁰³, and R¹⁰⁴ within the linker moiety are selected in such manner that: no two —C(═O)— moieties are adjected to each other; no two —O— or —NH— moieties are adjacent to each other; and/or no moieties are otherwise selected in an order such that an unstable molecule results (as defined as producing a molecule that has a shelf life at ambient temperature of less than about six months, five months, or four months) due to decomposition caused by the selection and order of R¹⁰⁰, R¹⁰¹, R¹⁰², R¹⁰³, and R¹⁰⁴.

The following are non-limiting examples of linker moieties and/or moieties that include a linker moiety in whole or in part that can be used in this disclosure. Based on this elaboration, those of skill in the art will understand how to use the full breadth of linker moieties that will accomplish the goal of the invention.

Non-limiting examples of moieties which may include the linker moiety, either in whole or in part, include, but are not limited to: a bond; —C(═O)—; —C≡C—; —NH—; —N(CH₃)—; —O—; —CH₂—; —(CH₂)₂—; —(CH₂)₃—; —(CH₂)₄—; —(CH₂)₅—; —(CH₂)₆—; —(CH₂)₇—; —(CH₂)₈—; —(CH₂)₉—; —(CH₂)₁₀—; —NH(C═O)—; —C(═O)NH—; —C(═O)CH₂—; —C(═O)(CH₂)₂—; —C(═O)(CH₂)₃—; —C(═O)(CH₂)₄—; —C(═O)(CH₂)₅—; —C(═O)(CH₂)₆—; —CH₂C(═O)—; —(CH₂)₂C(═O)—; —(CH₂)₃C(═O)—; —(CH₂)₄C(═O)—; —(CH₂)₅C(═O)—; —(CH₂)₆C(═O)—; —CH₂NH—; —(CH₂)₂NH—; —(CH₂)₃NH—; —(CH₂)₄NH—; —(CH₂)₅NH—; —(CH₂)₆NH—; —NHCH₂—; —NH(CH₂)₂—; —NH(CH₂)₃—; —NH(CH₂)₄—; —NH(CH₂)₅—; —NH(CH₂)₆—; —CH₂O—; —(CH₂)₂O—; —(CH₂)₃O—; —(CH₂)₄O—; —(CH₂)₅O—; —(CH₂)₆O—; —OCH₂—; —O(CH₂)₂—; —O(CH₂)₃—; —O(CH₂)₄—; —O(CH₂)₅—; —O(CH₂)₆—;

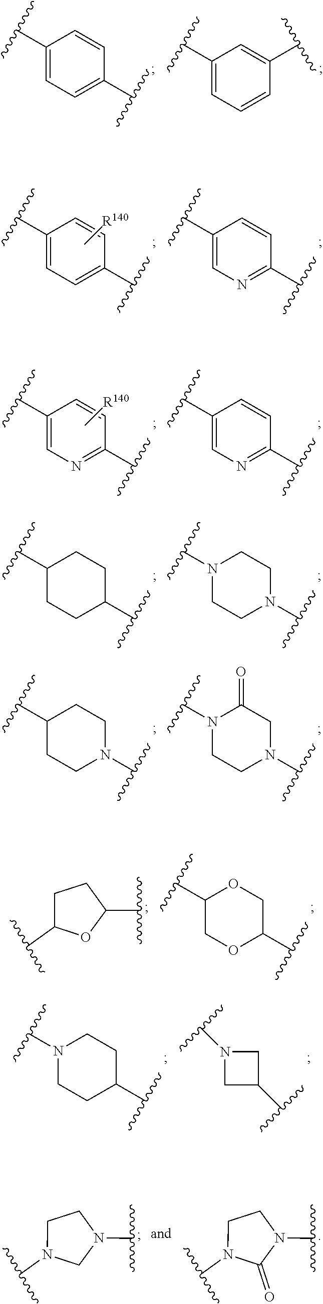

Further non-limiting examples of moieties which may include the linker moiety, either in whole or in part, include, but are not limited to:

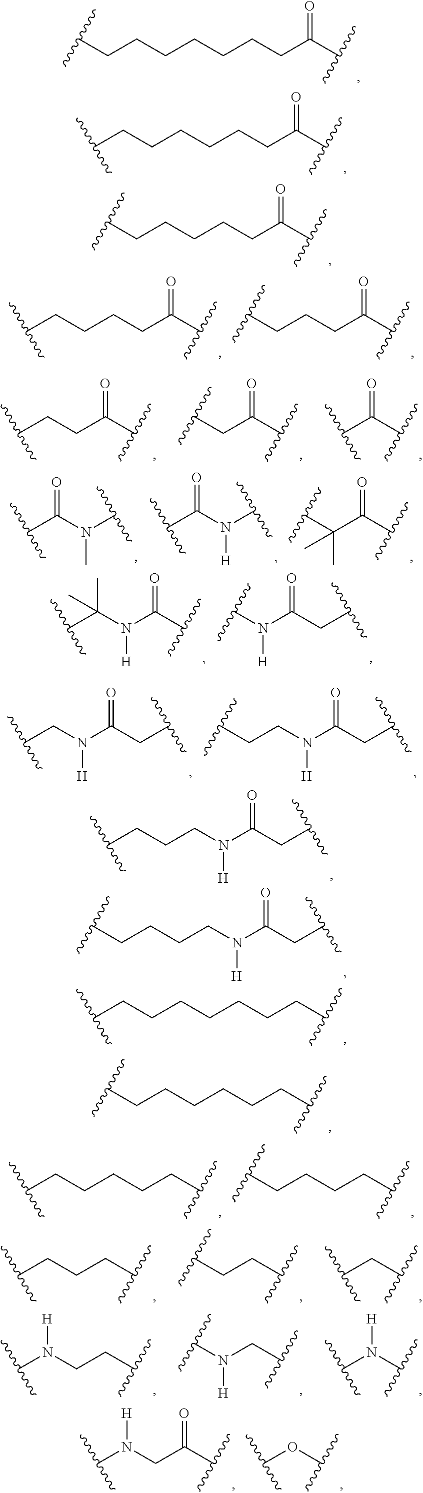

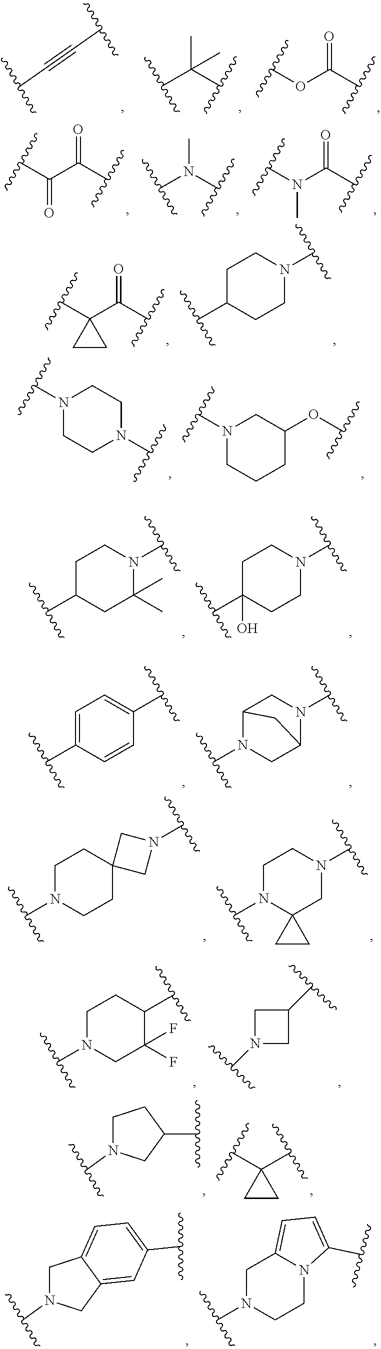

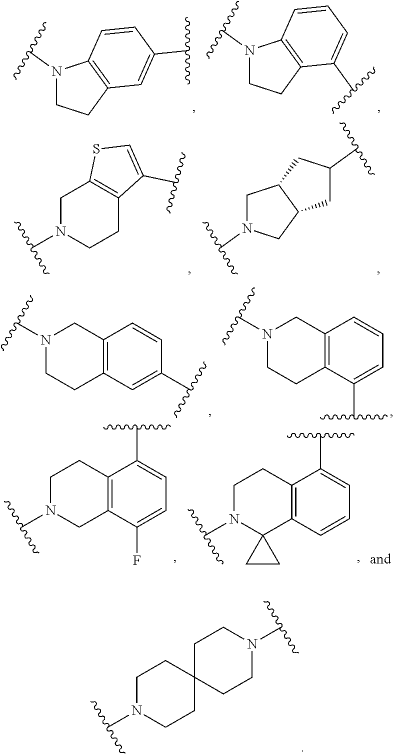

In some aspects, the linker moiety may include, either in whole or in part, a moiety selected from:

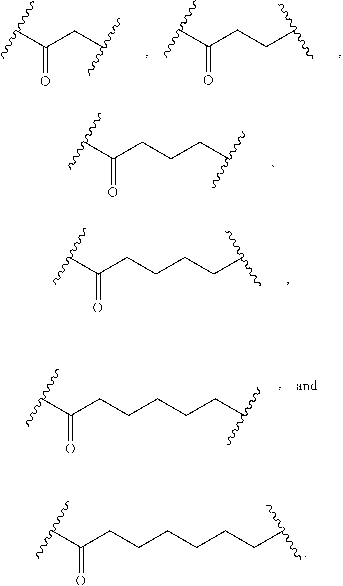 [SMILES: *CC(*)=O ; *CCC(*)=O ; *CCCC(*)=O ; *CCCCC(*)=O ; *CCCCCC(*)=O ; *CCCCCCC(*)=O]

In some aspects, the linker moiety may include, either in whole or in part, a moiety selected from:

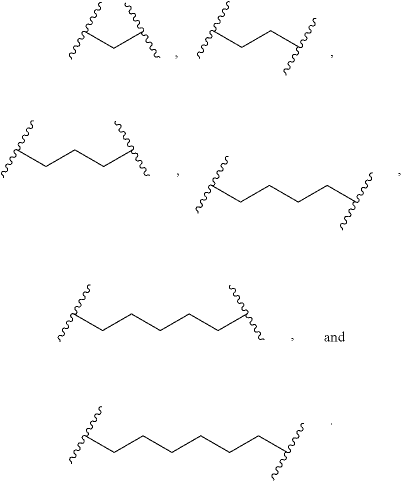

In some aspects, the linker moiety may include, either in whole or in part, a moiety selected from:

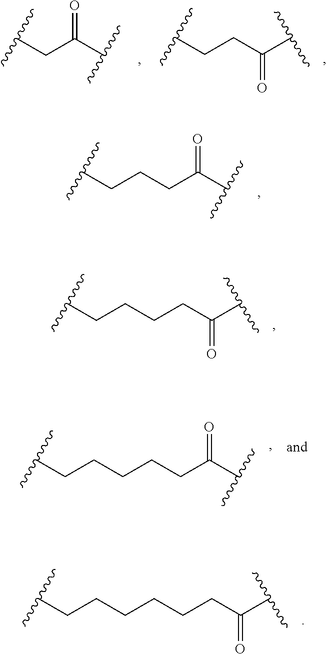 [SMILES: *CC(*)=O ; *CCC(*)=O ; *CCCC(*)=O ; *CCCCC(*)=O ; *CCCCCC(*)=O ; *CCCCCCC(*)=O]

In some aspects, the linker moiety may include, either in whole or in part,

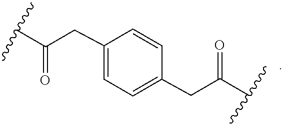 [SMILES: *C(=O)Cc1ccc(CC(*)=O)cc1]

In some aspects, the linker moiety may include, either in whole or in part,

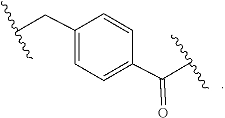 [SMILES: *Cc1ccc(C(*)=O)cc1]

In some aspects, the linker moiety may include, either in whole or in part, a moiety selected from:

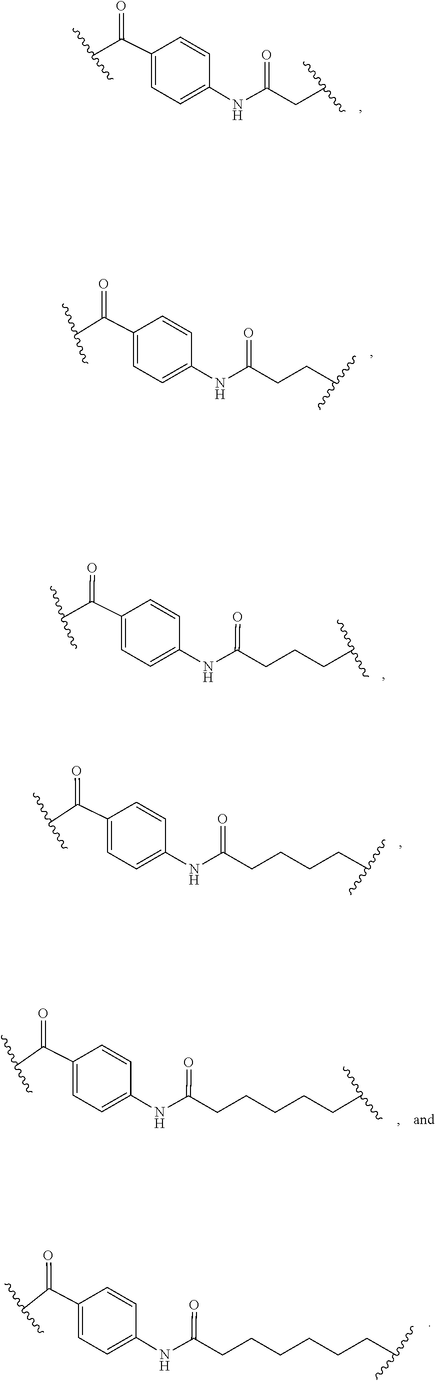 [SMILES: *CC(=O)Nc1ccc(C(*)=O)cc1 ; *CCC(=O)Nc1ccc(C(*)=O)cc1 ; *CCCC(=O)Nc1ccc(C(*)=O)cc1 ; *CCCCC(=O)Nc1ccc(C(*)=O)cc1 ; *CCCCCC(=O)Nc1ccc(C(*)=O)cc1 ; *CCCCCCC(=O)Nc1ccc(C(*)=O)cc1]

In some aspects, the linker moiety may include, either in whole or in part, a moiety selected from:

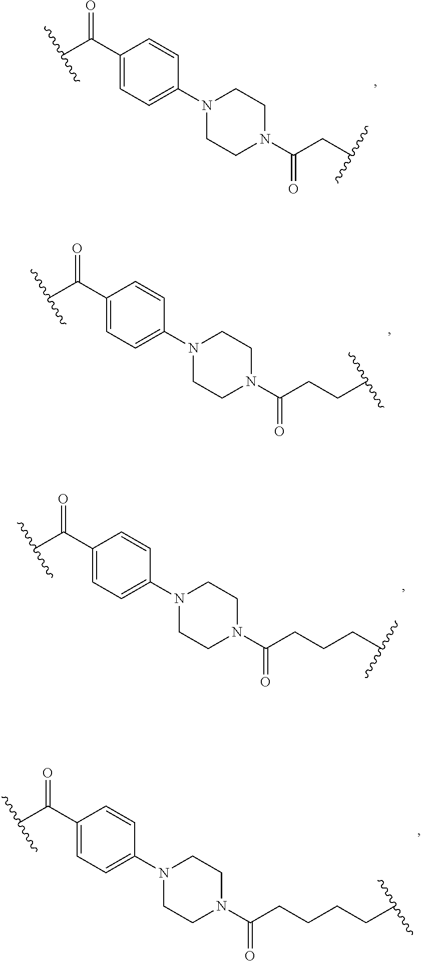

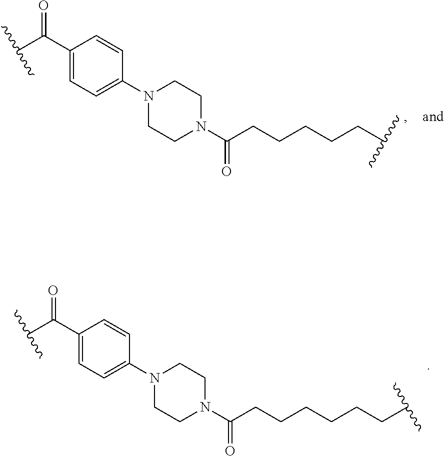 [SMILES: *CCCCCC(=O)N1CCN(c2ccc(C(*)=O)cc2)CC1 ; *CCCCCCC(=O)N1CCN(c2ccc(C(*)=O)cc2)CC1]

In some aspects, the linker moiety may include, either in whole or in part, a moiety selected from:

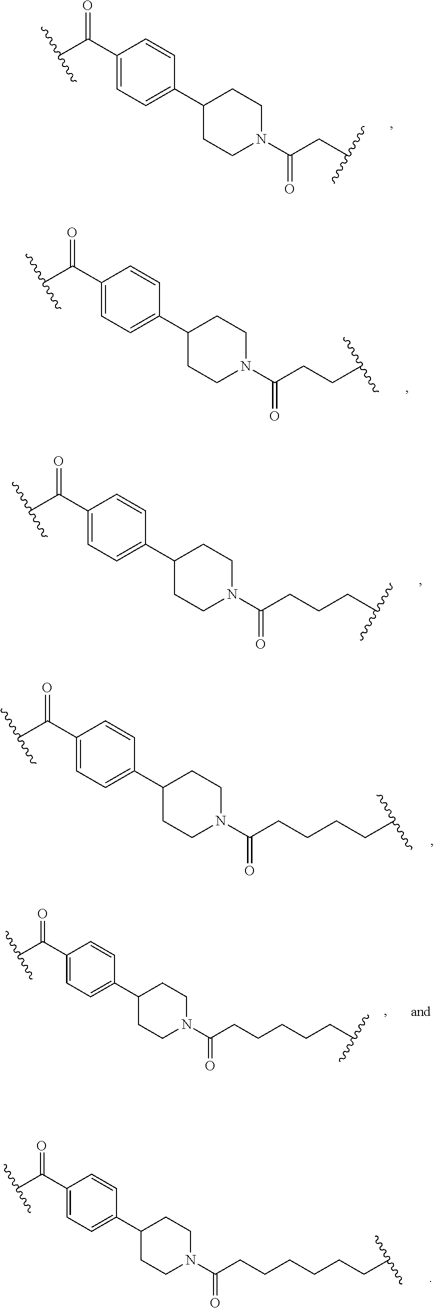 [SMILES: *CC(=O)N1CCC(c2ccc(C(*)=O)cc2)CC1 ; *CCC(=O)N1CCC(c2ccc(C(*)=O)cc2)CC1 ; *CCCC(=O)N1CCC(c2ccc(C(*)=O)cc2)CC1 ; *CCCCC(=O)N1CCC(c2ccc(C(*)=O)cc2)CC1 ; *CCCCCC(=O)N1CCC(c2ccc(C(*)=O)cc2)CC1 ; *CCCCCCC(=O)N1CCC(c2ccc(C(*)=O)cc2)CC1]

In some aspects, the linker moiety may include, either in whole or in part, a moiety selected from:

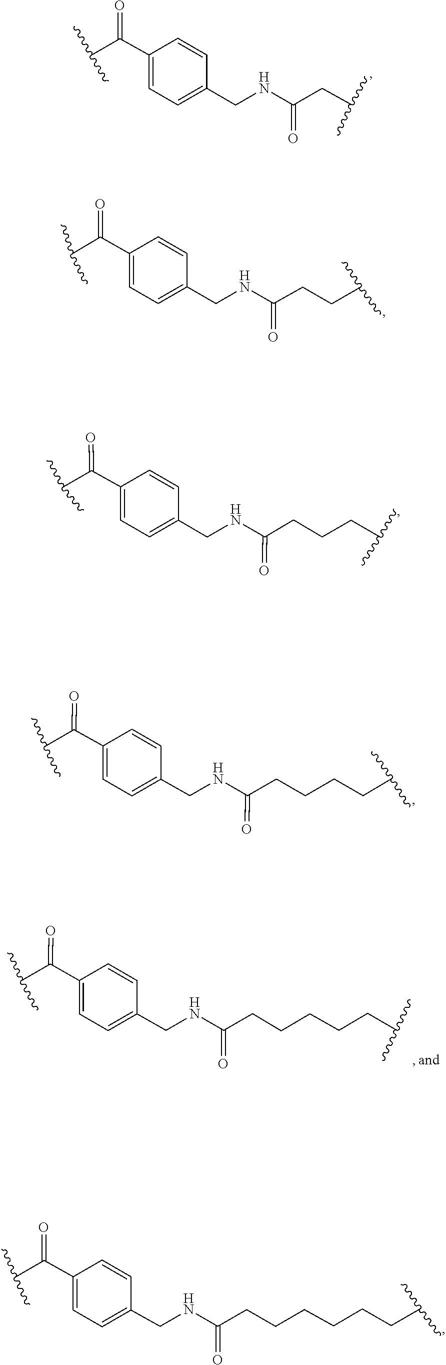 [SMILES: *CC(=O)NCc1ccc(C(*)=O)cc1 ; *CCC(=O)NCc1ccc(C(*)=O)cc1 ; *CCCC(=O)NCc1ccc(C(*)=O)cc1 ; *CCCCC(=O)NCc1ccc(C(*)=O)cc1 ; *CCCCCC(=O)NCc1ccc(C(*)=O)cc1 ; *CCCCCCC(=O)NCc1ccc(C(*)=O)cc1]

In some aspects, the linker moiety may include, either in whole or in part, a moiety selected from:

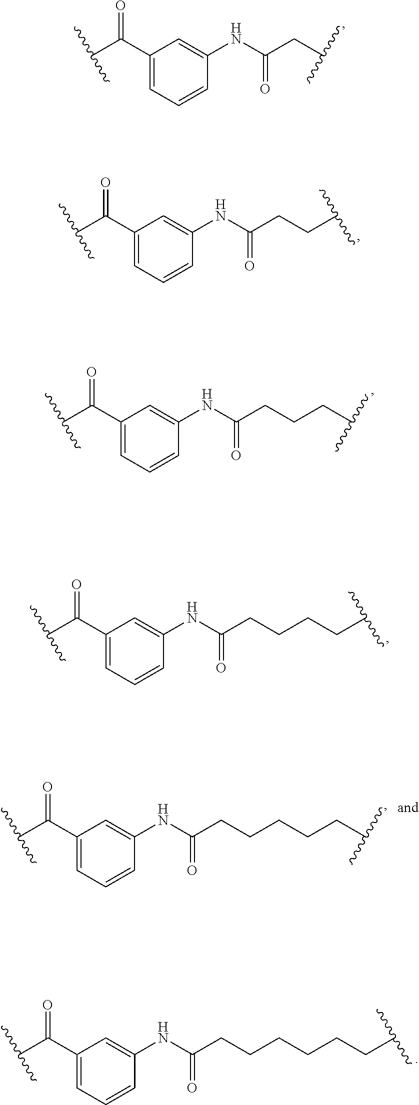 [SMILES: *CC(=O)Nc1cccc(C(*)=O)c1 ; *CCC(=O)Nc1cccc(C(*)=O)c1 ; *CCCC(=O)Nc1cccc(C(*)=O)c1 ; *CCCCC(=O)Nc1cccc(C(*)=O)c1 ; *CCCCCC(=O)Nc1cccc(C(*)=O)c1 ; *CCCCCCC(=O)Nc1cccc(C(*)=O)c1]

In some aspects, the linker moiety may include, either in whole or in part, a moiety selected from:

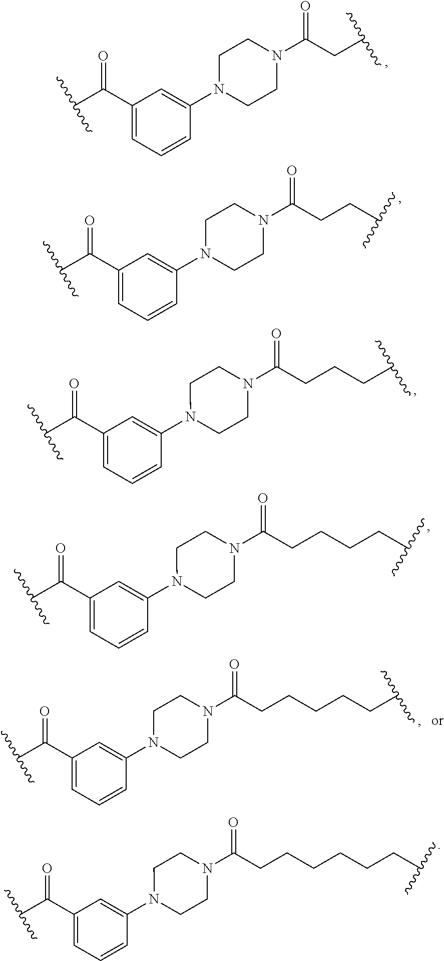 [SMILES: *CC(=O)N1CCN(c2cccc(C(*)=O)c2)CC1 ; *CCC(=O)N1CCN(c2cccc(C(*)=O)c2)CC1 ; *CCCC(=O)N1CCN(c2cccc(C(*)=O)c2)CC1 ; *CCCCC(=O)N1CCN(c2cccc(C(*)=O)c2)CC1 ; *CCCCCC(=O)N1CCN(c2cccc(C(*)=O)c2)CC1 ; *CCCCCCC(=O)N1CCN(c2cccc(C(*)=O)c2)CC1]

In some aspects, the linker moiety may include, either in whole or in part, a moiety selected from:

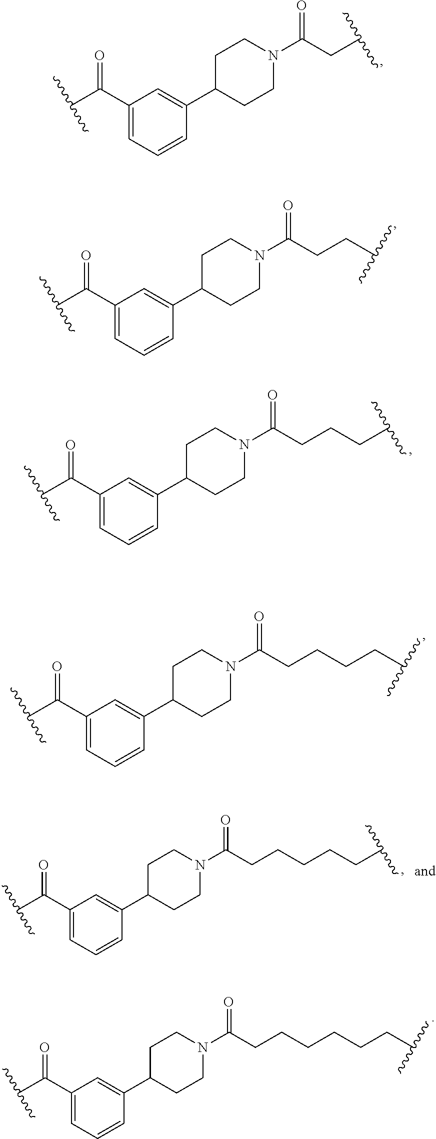 [SMILES: *CC(=O)N1CCC(c2cccc(C(*)=O)c2)CC1 ; *CCC(=O)N1CCC(c2cccc(C(*)=O)c2)CC1 ; *CCCC(=O)N1CCC(c2cccc(C(*)=O)c2)CC1 ; *CCCCC(=O)N1CCC(c2cccc(C(*)=O)c2)CC1 ; *CCCCCC(=O)N1CCC(c2cccc(C(*)=O)c2)CC1 ; *CCCCCCC(=O)N1CCC(c2cccc(C(*)=O)c2)CC1]

In some aspects, the linker moiety may include, either in whole or in part, a moiety selected from:

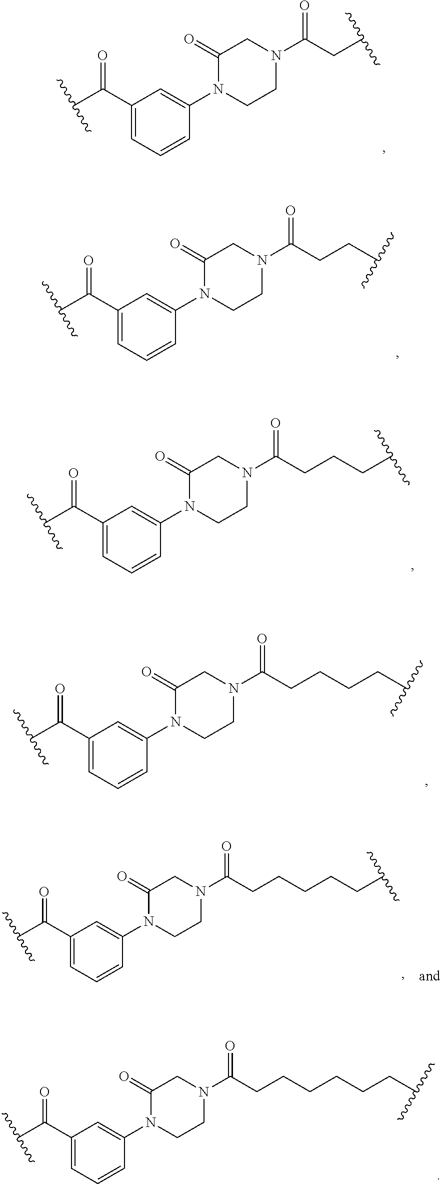 [SMILES: *CC(=O)N1CCN(c2cccc(C(*)=O)c2)C(=O)C1 ; *CCC(=O)N1CCN(c2cccc(C(*)=O)c2)C(=O)C1 ; *CCCC(=O)N1CCN(c2cccc(C(*)=O)c2)C(=O)C1 ; *CCCCC(=O)N1CCN(c2cccc(C(*)=O)c2)C(=O)C1 ; *CCCCCC(=O)N1CCN(c2cccc(C(*)=O)c2)C(=O)C1 ; *CCCCCCC(=O)N1CCN(c2cccc(C(*)=O)c2)C(=O)C1]

In some aspects, the linker moiety may include, either in whole or in part, a moiety selected from:

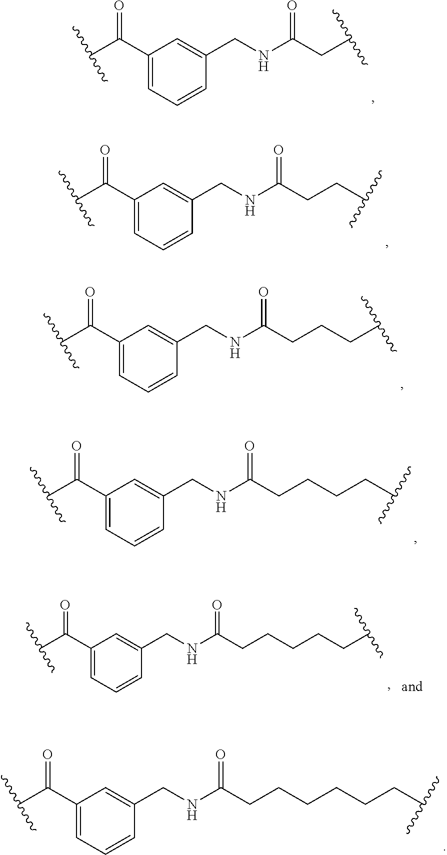 [SMILES: *CC(=O)NCc1cccc(C(*)=O)c1 ; *CCC(=O)NCc1cccc(C(*)=O)c1 ; *CCCC(=O)NCc1cccc(C(*)=O)c1 ; *CCCCC(=O)NCc1cccc(C(*)=O)c1 ; *CCCCCC(=O)NCc1cccc(C(*)=O)c1 ; *CCCCCCC(=O)NCc1cccc(C(*)=O)c1]

In some aspects, the linker moiety may include, either in whole or in part, a moiety selected from:

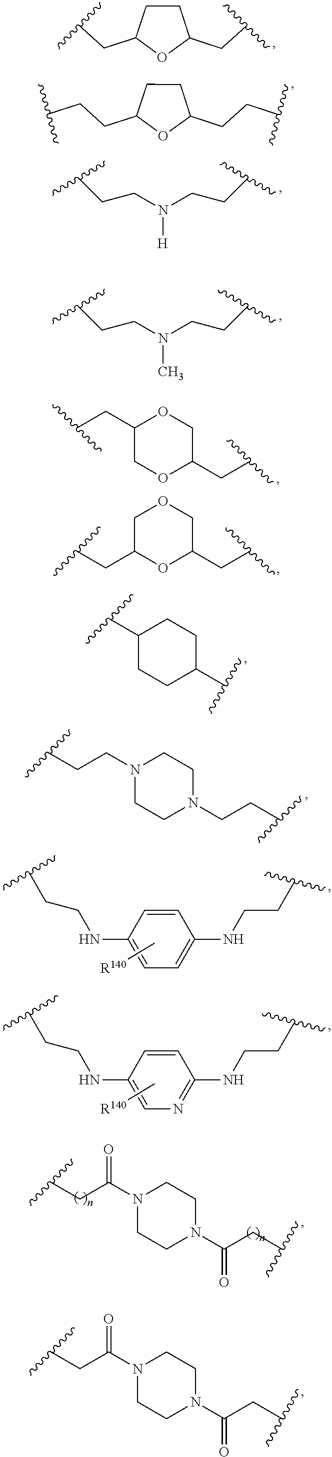

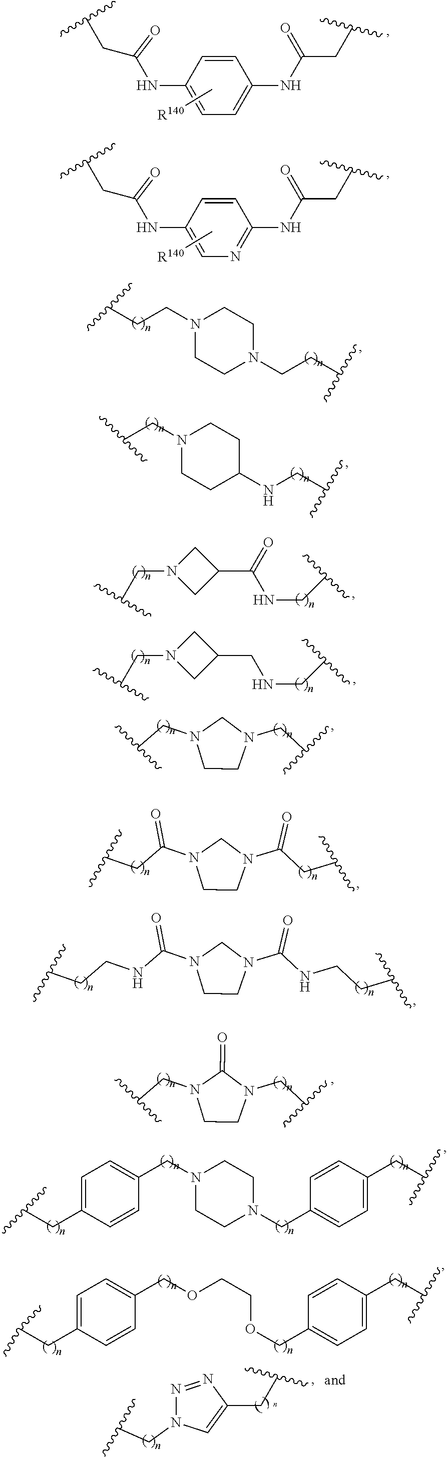

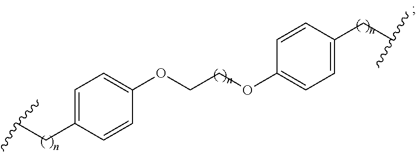 [SMILES: *Cc1ccc(OCCOc2ccc(C*)cc2)cc1]

wherein n in the above formulae is independently selected at each occurrence from 1, 2, 3, 4, 5, and 6; and all other variables are as defined herein.

In some aspects, the linker moiety may include, either in whole or in part, a moiety selected from:

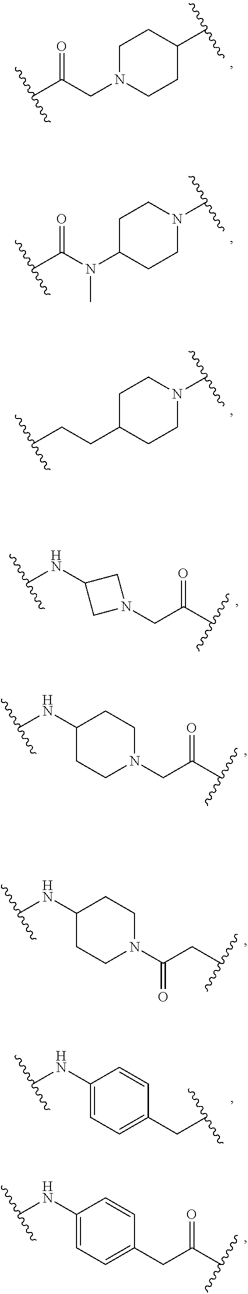

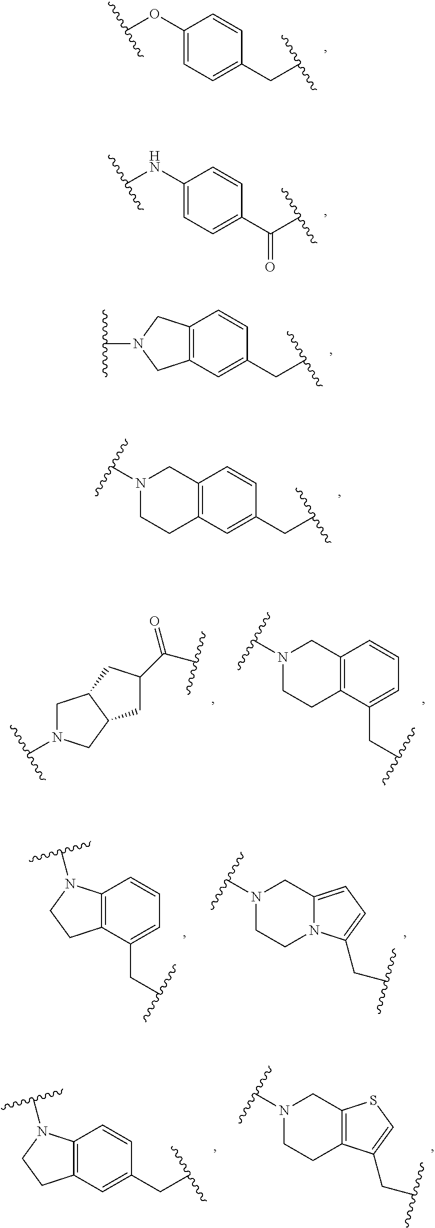

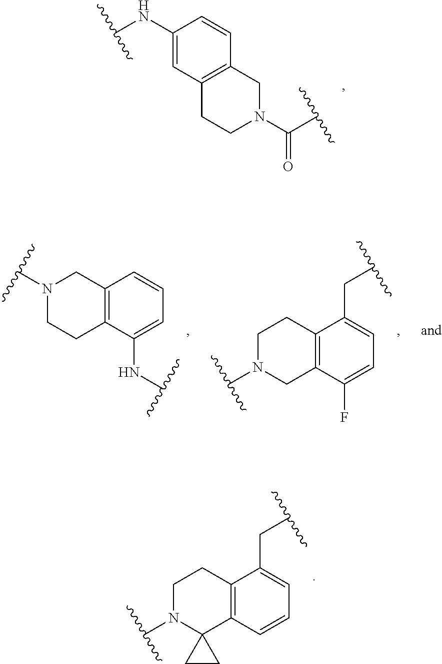 [SMILES: *Nc1ccc2c(c1)CCN(C(*)=O)C2 ; *Nc1cccc2c1CCN(*)C2 ; *Cc1ccc(F)c2c1CCN(*)C2 ; *Cc1cccc2c1CCN(*)C21CC1]

In some aspects, the linker moiety may include, either in whole or in part, a moiety selected from:

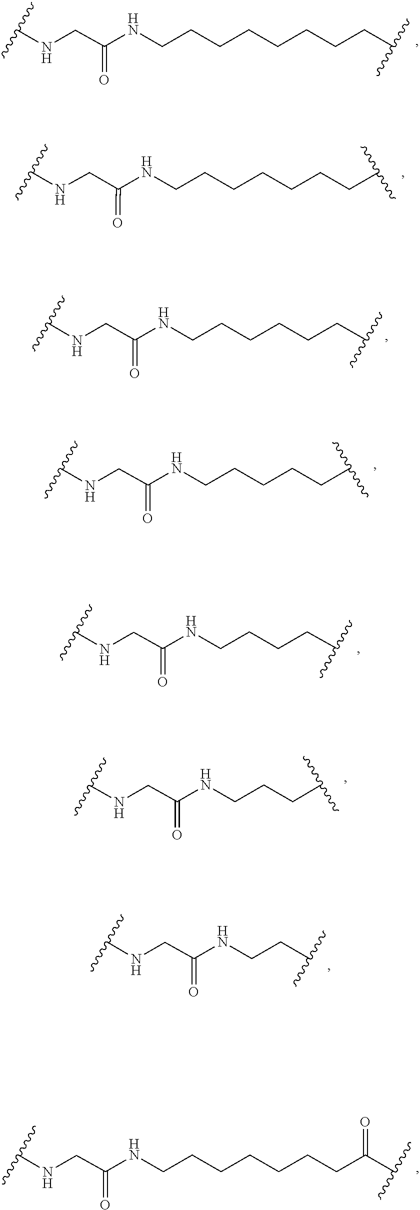

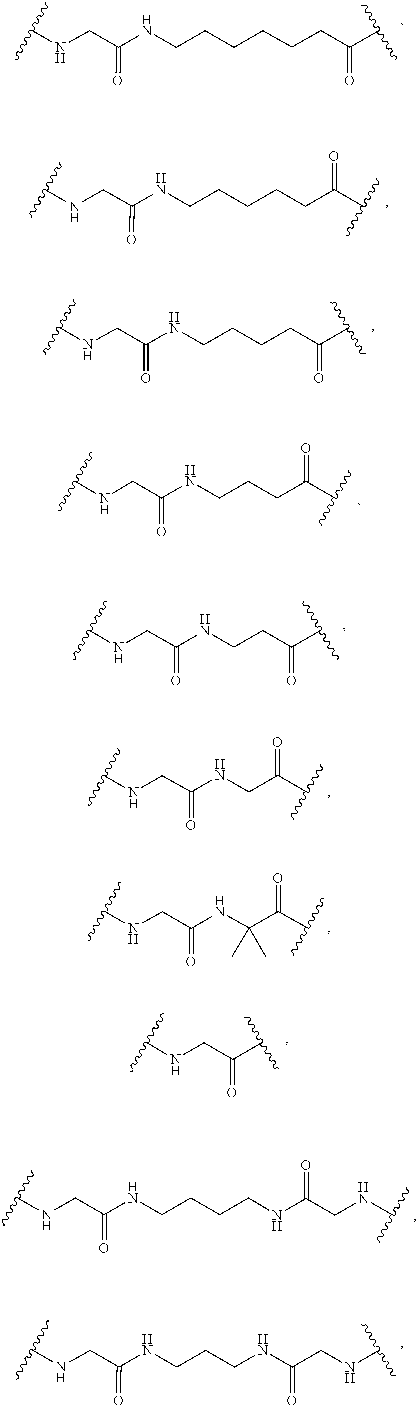

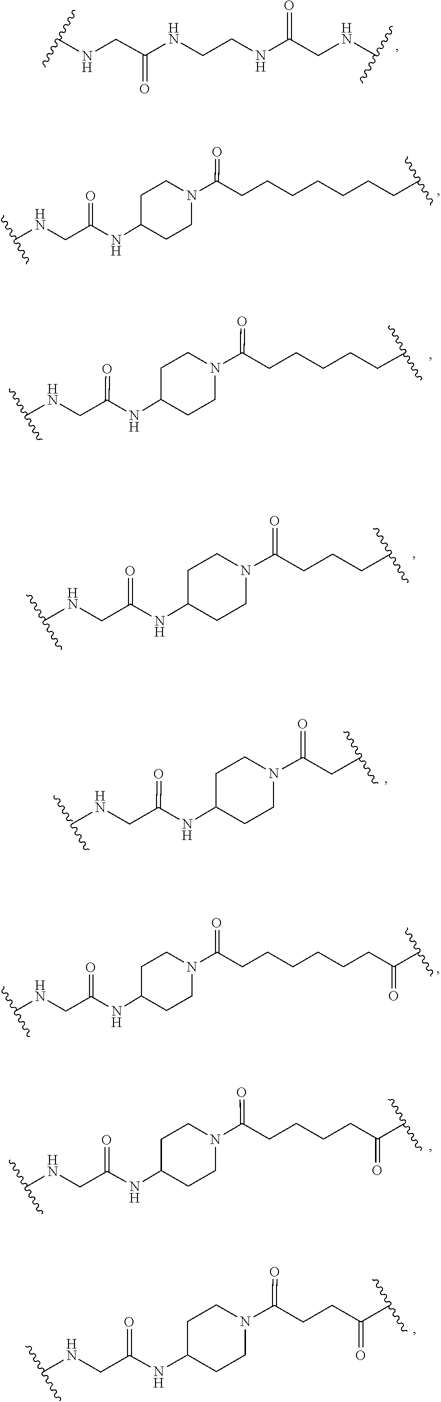

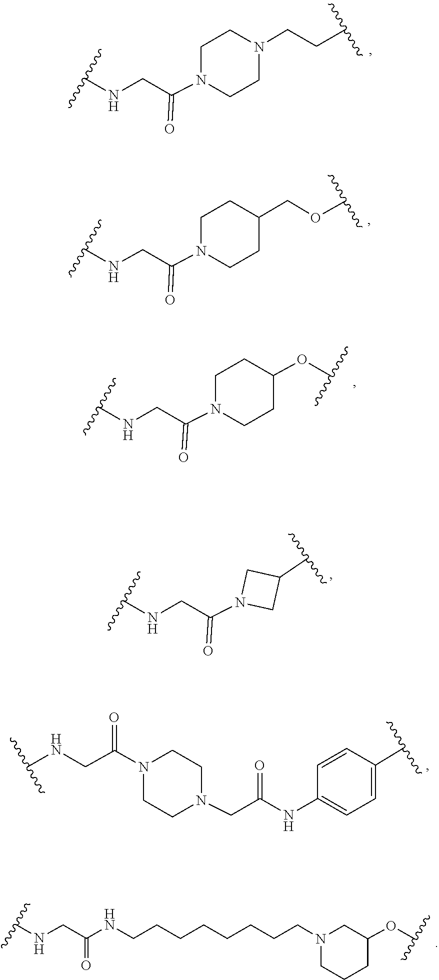 [SMILES: *CCN1CCN(C(=O)CN*)CC1 ; *NCC(=O)N1CCC(CO*)CC1 ; *NCC(=O)N1CCC(O*)CC1 ; *NCC(=O)N1CC(*)C1 ; *NCC(=O)N1CCN(CC(=O)Nc2ccc(*)cc2)CC1 ; *NCC(=O)NCCCCCCCCN1CCCC(O*)C1]

In some aspects, the linker moiety may include, either in whole or in part, a moiety selected from:

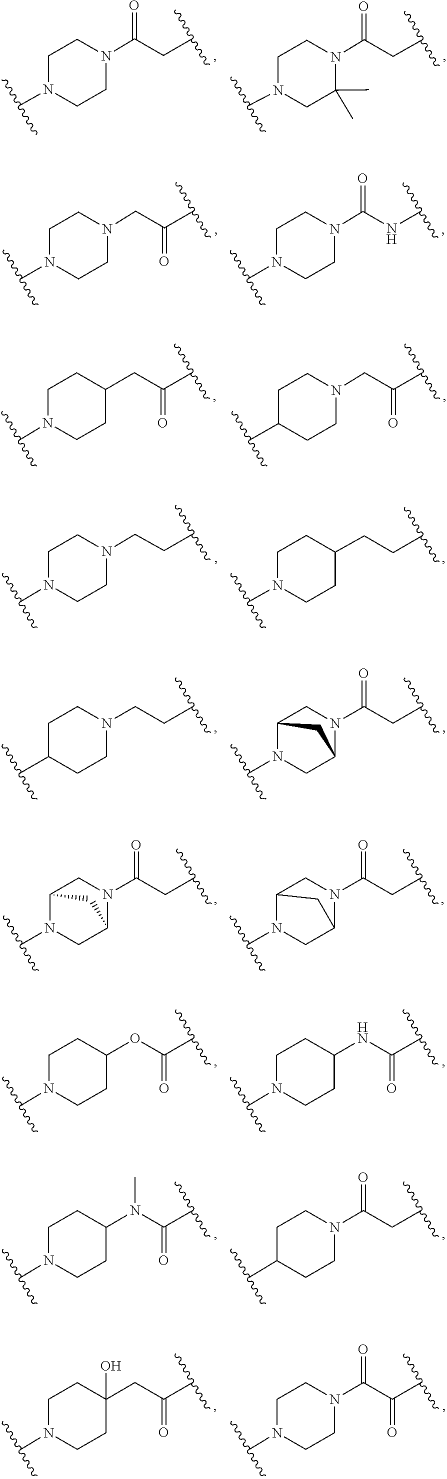

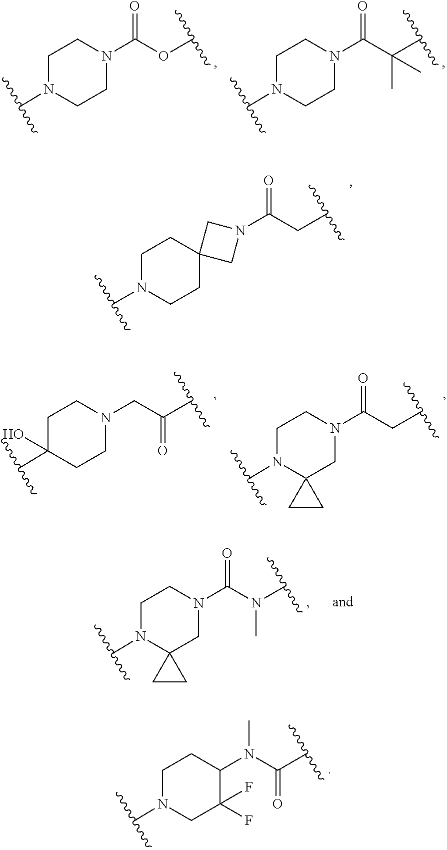

In some aspects, the linker moiety may include, either in whole or in part, a moiety selected from:

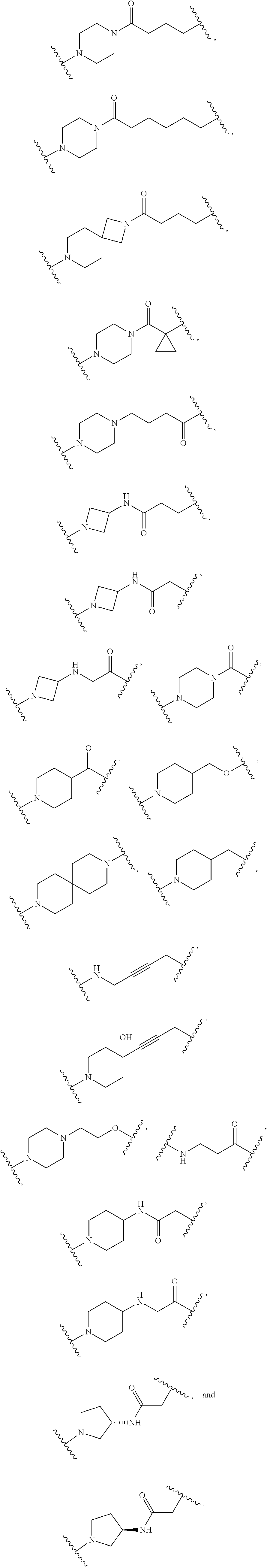

In some aspects, the linker moiety may include, either in whole or in part, a moiety selected from:

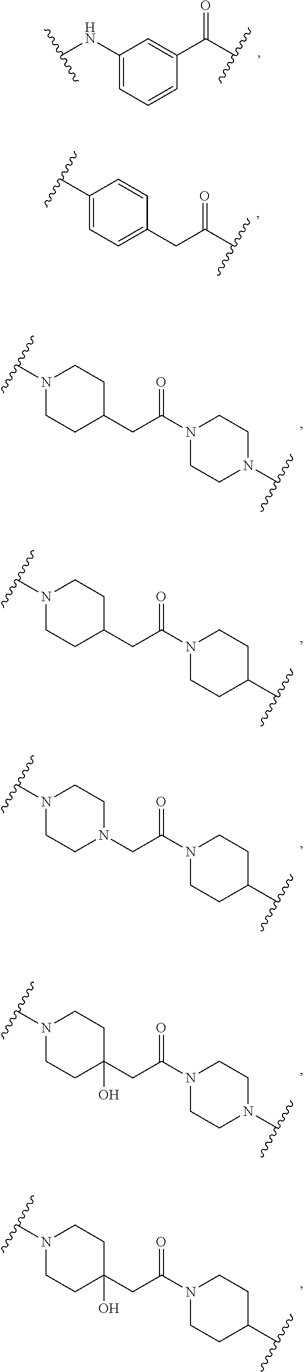

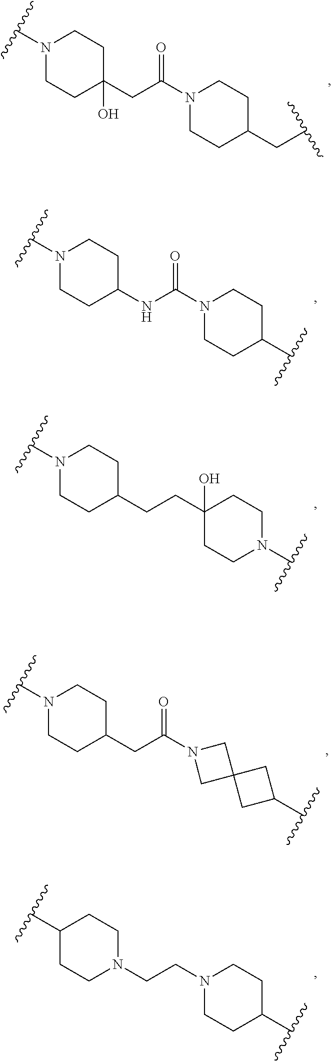 [SMILES: *CC1CCN(C(=O)CC2(O)CCN(*)CC2)CC1 ; *C1CCN(C(=O)NC2CCN(*)CC2)CC1 ; *N1CCC(CCC2(O)CCN(*)CC2)CC1 ; *C1CC2(C1)CN(C(=O)CC1CCN(*)CC1)C2 ; *C1CCN(CCN2CCC(*)CC2)CC1]

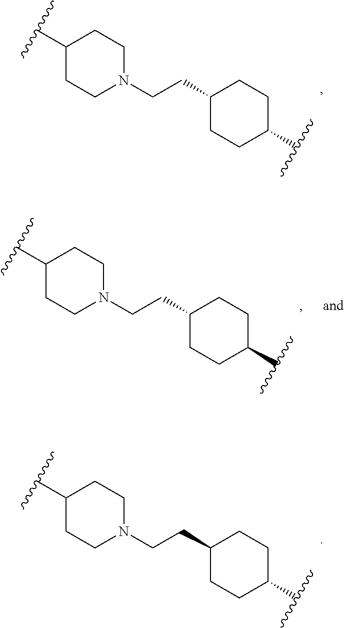 [SMILES: *C1CCN(CC[C@H]2CC[C@@H](*)CC2)CC1 ; *C1CCN(CC[C@H]2CC[C@H](*)CC2)CC1 ; *C1CCN(CC[C@H]2CC[C@H](*)CC2)CC1]

### Pharmaceutical Compositions

The compounds described herein can be administered by any suitable method and technique presently or prospectively known to those skilled in the art. For example, the active components described herein can be formulated in a physiologically- or pharmaceutically-acceptable form and administered by any suitable route known in the art including, for example, oral and parenteral routes of administering. As used herein, the term “parenteral” includes subcutaneous, intradermal, intravenous, intramuscular, intraperitoneal, and intrasternal administration, such as by injection. Administration of the active components of their compositions can be a single administration, or at continuous and distinct intervals as can be readily determined by a person skilled in the art.

Compositions, as described herein, comprising an active compound and a pharmaceutically acceptable carrier or excipient of some sort may be useful in a variety of medical and non-medical applications. For example, pharmaceutical compositions comprising an active compound and an excipient may be useful for the treatment or prevention of a cancer in a subject in need thereof.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Excipients” include any and all solvents, diluents or other liquid vehicles, dispersion or suspension aids, surface active agents, isotonic agents, thickening or emulsifying agents, preservatives, solid binders, lubricants and the like, as suited to the particular dosage form desired. General considerations in formulation and/or manufacture can be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005).

Exemplary excipients include, but are not limited to, any non-toxic, inert solid, semisolid or liquid filler, diluent, encapsulating material or formulation auxiliary of any type. Some examples of materials which can serve as excipients include, but are not limited to, sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; tale; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. As would be appreciated by one of skill in this art, the excipients may be chosen based on what the composition is useful for. For example, with a pharmaceutical composition or cosmetic composition, the choice of the excipient will depend on the route of administration, the agent being delivered, time course of delivery of the agent, etc., and can be administered to humans and/or to animals, orally, rectally, parenterally, intracisternally, intravaginally, intranasally, intraperitoneally, topically (as by powders, creams, ointments, or drops), buccally, or as an oral or nasal spray. In some aspects, the active compounds disclosed herein are administered topically.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Exemplary granulating and/or dispersing agents include potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. Exemplary binding agents include starch (e.g. cornstarch and starch paste), gelatin, sugars (e.g. sucrose, glucose, dextrose, dextrin, molasses, lactose, lactitol, mannitol, etc.), natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, etc., and/or combinations thereof.

Exemplary preservatives include antioxidants, chelating agents, antimicrobial preservatives, antifungal preservatives, alcohol preservatives, acidic preservatives, and other preservatives.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid. Other preservatives include tocopherol, tocopherol acetate, deteroxime mesylate, cetrimide, butylated hydroxyanisol (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain aspects, the preservative is an antioxidant. In other aspects, the preservative is a chelating agent.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and combinations thereof.

Exemplary lubricating agents include magnesium stearate, calcium stearate, stearic acid, silica, tale, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary natural oils include almond, apricot kernel, avocado, babassu, bergamot, black current seed, borage, cade, chamomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, Litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

Additionally, the composition may further comprise a polymer. Exemplary polymers contemplated herein include, but are not limited to, cellulosic polymers and copolymers, for example, cellulose ethers such as methylcellulose (MC), hydroxyethylcellulose (HEC), hydroxypropyl cellulose (HPC), hydroxypropyl methyl cellulose (HPMC), methylhydroxyethylcellulose (MHEC), methylhydroxypropylcellulose (MHPC), carboxymethyl cellulose (CMC) and its various salts, including, e.g., the sodium salt, hydroxyethylcarboxymethylcellulose (HECMC) and its various salts, carboxymethylhydroxyethylcellulose (CMHEC) and its various salts, other polysaccharides and polysaccharide derivatives such as starch, dextran, dextran derivatives, chitosan, and alginic acid and its various salts, carageenan, varoius gums, including xanthan gum, guar gum, gum arabic, gum karaya, gum ghatti, konjac and gum tragacanth, glycosaminoglycans and proteoglycans such as hyaluronic acid and its salts, proteins such as gelatin, collagen, albumin, and fibrin, other polymers, for example, polyhydroxyacids such as polylactide, polyglycolide, polyl(lactide-co-glycolide) and poly(.epsilon.-caprolactone-co-glycolide)-, carboxyvinyl polymers and their salts (e.g., carbomer), polyvinylpyrrolidone (PVP), polyacrylic acid and its salts, polyacrylamide, polyacrylic acid/acrylamide copolymer, polyalkylene oxides such as polyethylene oxide, polypropylene oxide, poly(ethylene oxide-propylene oxide), and a Pluronic polymer, polyoxy ethylene (polyethylene glycol), polyanhydrides, polyvinylalchol, polyethyleneamine and polypyrridine, polyethylene glycol (PEG) polymers, such as PEGylated lipids (e.g., PEG-stearate, 1,2-Distearoyl-sn-glycero-3-Phosphoethanolamine-N-[Methoxy(Polyethylene glycol)-1000], 1,2-Distearoyl-sn-glycero-3-Phosphoethanolamine-N-[Methoxy(Polyethylene glycol)-2000], and 1,2-Distearoyl-sn-glycero-3-Phosphoethanolamine-N-[Methoxy(Polyethylene glycol)-5000]), copolymers and salts thereof.

Additionally, the composition may further comprise an emulsifying agent. Exemplary emulsifying agents include, but are not limited to, a polyethylene glycol (PEG), a polypropylene glycol, a polyvinyl alcohol, a poly-N-vinyl pyrrolidone and copolymers thereof, poloxamer nonionic surfactants, neutral water-soluble polysaccharides (e.g., dextran, Ficoll, celluloses), non-cationic poly(meth)acrylates, non-cationic polyacrylates, such as poly (meth) acrylic acid, and esters amide and hydroxy alkyl amides thereof, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. In certain aspects, the emulsifying agent is cholesterol.

Liquid compositions include emulsions, microemulsions, solutions, suspensions, syrups, and elixirs. In addition to the active compound, the liquid composition may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable compositions, for example, injectable aqueous or oleaginous suspensions may be formulated according to the known art using suitable dispersing or wetting agents and suspending agents. The sterile injectable preparation may also be a injectable solution, suspension, or emulsion in a nontoxic parenterally acceptable diluent or solvent, for example, as a solution in 1,3-butanediol. Among the acceptable vehicles and solvents for pharmaceutical or cosmetic compositions that may be employed are water, Ringer's solution, U.S.P. and isotonic sodium chloride solution. In addition, sterile, fixed oils are conventionally employed as a solvent or suspending medium. Any bland fixed oil can be employed including synthetic mono- or diglycerides. In addition, fatty acids such as oleic acid are used in the preparation of injectables. In certain aspects, the particles are suspended in a carrier fluid comprising 1% (w/v) sodium carboxymethyl cellulose and 0.1% (v/v) Tween 80. The injectable composition can be sterilized, for example, by filtration through a bacteria-retaining filter, or by incorporating sterilizing agents in the form of sterile solid compositions which can be dissolved or dispersed in sterile water or other sterile injectable medium prior to use.

Compositions for rectal or vaginal administration may be in the form of suppositories which can be prepared by mixing the particles with suitable non-irritating excipients or carriers such as cocoa butter, polyethylene glycol, or a suppository wax which are solid at ambient temperature but liquid at body temperature and therefore melt in the rectum or vaginal cavity and release the particles.

Solid compositions include capsules, tablets, pills, powders, and granules. In such solid compositions, the particles are mixed with at least one excipient and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as tale, calcium stearate, magnesium stearate, solid polyethylene glycols, sodium lauryl sulfate, and mixtures thereof. In the case of capsules, tablets, and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

Tablets, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. Solid compositions of a similar type may also be employed as fillers in soft and hard-filled gelatin capsules using such excipients as lactose or milk sugar as well as high molecular weight polyethylene glycols and the like.

Compositions for topical or transdermal administration include ointments, pastes, creams, lotions, gels, powders, solutions, sprays, inhalants, or patches. The active compound is admixed with an excipient and any needed preservatives or buffers as may be required.

The ointments, pastes, creams, and gels may contain, in addition to the active compound, excipients such as animal and vegetable fats, oils, waxes, paraffins, starch, tragacanth, cellulose derivatives, polyethylene glycols, silicones, bentonites, silicic acid, tale, and zinc oxide, or mixtures thereof.

Powders and sprays can contain, in addition to the active compound, excipients such as lactose, tale, silicic acid, aluminum hydroxide, calcium silicates, and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the nanoparticles in a proper medium. Absorption enhancers can also be used to increase the flux of the compound across the skin. The rate can be controlled by either providing a rate controlling membrane or by dispersing the particles in a polymer matrix or gel.

### Methods of Use

In another aspect, methods of treating medical disorders in a subject in need thereof are provided. In some aspects, the medical disorder can be mediated by a target protein as described herein. In some aspects, the method can include administering a PROTAC or pharmaceutical composition as described herein. In some aspects, the target protein can be degraded or downregulated by the PROTAC upon administration.

In some aspects, the medical disorder can include a cancer. The term “neoplasia” or “cancer” is used throughout this disclosure to refer to the pathological process that results in the formation and growth of a cancerous or malignant neoplasm, i.e., abnormal tissue (solid) or cells (non-solid) that grow by cellular proliferation, often more rapidly than normal and continues to grow after the stimuli that initiated the new growth cease. Malignant neoplasms show partial or complete lack of structural organization and functional coordination with the normal tissue and most invade surrounding tissues, can metastasize to several sites, are likely to recur after attempted removal and may cause the death of the patient unless adequately treated. As used herein, the term neoplasia is used to describe all cancerous disease states and embraces or encompasses the pathological process associated with malignant, hematogenous, ascitic and solid tumors. The cancers which may be treated by the compositions disclosed herein may comprise carcinomas, sarcomas, lymphomas, leukemias, germ cell tumors, or blastomas.

Carcinomas that may be treated by the compositions of the present disclosure include, but are not limited to, acinar carcinoma, acinous carcinoma, alveolar adenocarcinoma, carcinoma adenomatosum, adenocarcinoma, carcinoma of adrenal cortex, alveolar carcinoma, alveolar cell carcinoma, basal cell carcinoma, carcinoma basocellular, basaloid carcinoma, basosquamous cell carcinoma, breast carcinoma, bronchioalveolar carcinoma, bronchiolar carcinoma, cerebriform carcinoma, cholangiocellular carcinoma, chorionic carcinoma, colloid carcinoma, comedocarcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epibulbar carcinoma, epidermoid carcinoma, carcinoma epitheliate adenoids, carcinoma exulcere, carcinoma fibrosum, gelatinform carcinoma, gelatinous carcinoma, giant cell carcinoma, gigantocellulare, glandular carcinoma, granulose cell carcinoma, hair matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, lentivular carcinoma, carcinoma lenticulare, lipomatous carcinoma, lymphoepithelial carcinoma, carcinoma mastotoids, carcinoma medullare, medullary carcinoma, carcinoma melanodes, melanotonic carcinoma, mucinous carcinoma, carcinoma muciparum, carcinoma mucocullare, mucoepidermoid carcinoma, mucous carcinoma, carcinoma myxomatodes, masopharyngeal carcinoma, carcinoma nigrum, oat cell carcinoma, carcinoma ossificans, osteroid carcinoma, ovarian carcinoma, papillary carcinoma, periportal carcinoma, preinvasive carcinoma, prostate carcinoma, renal cell carcinoma of kidney, reserve cell carcinoma, carcinoma sarcomatodes, scheinderian carcinoma, scirrhous carcinoma, carcinoma scrota, signet-ring cell carcinoma, carcinoma simplex, small cell carcinoma, solandoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberrosum, tuberous carcinoma, verrucous carcinoma, and carcinoma vilosum.

Representative sarcomas that may be treated by the compositions of the present disclosure include, but are not limited to, liposarcomas (including myxoid liposarcomas and pleomorphic liposarcomas), leiomyosarcomas, rhabdomyosarcomas, neurofibrosarcomas, malignant peripheral nerve sheath tumors, Ewing's tumors (including Ewing's sarcoma of bone, extraskeletal or non-bone) and primitive neuroectodermal tumors (PNET), synovial sarcoma, hemangioendothelioma, fibrosarcoma, desmoids tumors, dermatofibrosarcoma protuberance (DFSP), malignant fibrous histiocytoma (MFH), hemangiopericytoma, malignant mesenchymoma, alveolar soft-part sarcoma, epithelioid sarcoma, clear cell sarcoma, desmoplastic small cell tumor, gastrointestinal stromal tumor (GIST) and osteosarcoma (also known as osteogenic sarcoma) skeletal and extra-skeletal, and chondrosarcoma.

The compositions of the present disclosure may be used in the treatment of a lymphoma. Lymphomas that may be treated include mature B cell neoplasms, mature T cell and natural killer (NK) cell neoplasms, precursor lymphoid neoplasms, Hodgkin lymphomas, and immunodeficiency-associated lymphoproliferative disorders. Representative mature B cell neoplasms include, but are not limited to, B-cell chronic lymphocytic leukemia/small cell lymphoma, B-cell prolymphocytic leukemia, lymphoplasmacytic lymphoma (such as Waldenström macroglobulinemia), splenic marginal zone lymphoma, hairy cell leukemia, plasma cell neoplasms (such as plasma cell myeloma/multiple myeloma, plasmacytoma, monoclonal immunoglobulin deposition diseases, and heavy chain diseases), extranodal marginal zone B cell lymphoma (MALT lymphoma), nodal marginal zone B cell lymphoma, follicular lymphoma, primary cutaneous follicular center lymphoma, mantle cell lymphoma, diffuse large B cell lymphoma, diffuse large B-cell lymphoma associated with chronic inflammation, Epstein-Barr virus-positive DLBCL of the elderly, lyphomatoid granulomatosis, primary mediastinal (thymic) large B-cell lymphoma, intravascular large B-cell lymphoma, ALK+ large B-cell lymphoma, plasmablastic lymphoma, primary effusion lymphoma, large B-cell lymphoma arising in HHV8-associated multicentric Castleman's disease, and Burkitt lymphoma/leukemia. Representative mature T cell and NK cell neoplasms include, but are not limited to, T-cell prolymphocytic leukemia, T-cell large granular lymphocyte leukemia, aggressive NK cell leukemia, adult T-cell leukemia/lymphoma, extranodal NK/T-cell lymphoma, nasal type, enteropathy-associated T-cell lymphoma, hepatosplenic T-cell lymphoma, blastic NK cell lymphoma, lycosis fungoides/Sezary syndrome, primary cutaneous CD30-positive T cell lymphoproliferative disorders (such as primary cutaneous anaplastic large cell lymphoma and lymphomatoid papulosis), peripheral T-cell lymphoma not otherwise specified, angioimmunoblastic T cell lymphoma, and anaplastic large cell lymphoma. Representative precursor lymphoid neoplasms include B-lymphoblastic leukemia/lymphoma not otherwise specified, B-lymphoblastic leukemia/lymphoma with recurrent genetic abnormalities, or T-lymphoblastic leukemia/lymphoma. Representative Hodgkin lymphomas include classical Hodgkin lymphomas, mixed cellularity Hodgkin lymphoma, lymphocyte-rich Hodgkin lymphoma, and nodular lymphocyte-predominant Hodgkin lymphoma.

The compositions of the present disclosure may be used in the treatment of a Leukemia. Representative examples of leukemias that may be treated include, but are not limited to, acute lymphoblastic leukemia (ALL), chronic lymphocytic leukemia (CLL), acute myelogenous leukemia (AML), chronic myelogenous leukemia (CML), hairy cell leukemia (HCL), T-cell prolymphocytic leukemia, adult T-cell leukemia, clonal eosinophilias, and transient myeloproliferative disease.

The compositions of the present disclosure may be used in the treatment of a germ cell tumor, for example germinomatous (such as germinoma, dysgerminoma, and seminoma), non germinomatous (such as embryonal carcinoma, endodermal sinus tumor, choriocarcinoma, teratoma, polyembryoma, and gonadoblastoma). and mixed tumors.

The compositions of the present disclosure may be used in the treatment of blastomas, for example hepatoblastoma, medulloblastoma, nephroblastoma, neuroblastoma, pancreatoblastoma, pleuropulmonary blastoma, retinoblastoma, and glioblastoma multiforme.

Representative cancers that may be treated include, but are not limited to: bone and muscle sarcomas such as chondrosarcoma, Ewing's sarcoma, malignant fibrous histiocytoma of bone/osteosarcoma, osteosarcoma, rhabdomyosarcoma, and heart cancer; brain and nervous system cancers such as astrocytoma, brainstem glioma, pilocytic astrocytoma, ependymoma, primitive neuroectodermal tumor, cerebellar astrocytoma, cerebral astrocytoma, glioma, medulloblastoma, neuroblastoma, oligodendroglioma, pineal astrocytoma, pituitary adenoma, and visual pathway and hypothalamic glioma; breast cancers including invasive lobular carcinoma, tubular carcinoma, invasive cribriform carcinoma, medullary carcinoma, male breast cancer, Phyllodes tumor, and inflammatory breast cancer; endocrine system cancers such as adrenocortical carcinoma, islet cell carcinoma, multiple endocrine neoplasia syndrome, parathyroid cancer, phemochromocytoma, thyroid cancer, and Merkel cell carcinoma; eye cancers including uveal melanoma and retinoblastoma; gastrointestinal cancers such as anal cancer, appendix cancer, cholangiocarcinoma, gastrointestinal carcinoid tumors, colon cancer, extrahepatic bile duct cancer, gallbladder cancer, gastric cancer, gastrointestinal stromal tumor, hepatocellular cancer, pancreatic cancer, and rectal cancer; genitourinary and gynecologic cancers such as bladder cancer, cervical cancer, endometrial cancer, extragonadal germ cell tumor, ovarian cancer, ovarian epithelial cancer, ovarian germ cell tumor, penile cancer, renal cell carcinoma, renal pelvis and ureter transitional cell cancer, prostate cancer, testicular cancer, gestational trophoblastic tumor, urethral cancer, uterine sarcoma, vaginal cancer, vulvar cancer, and Wilms tumor; head and neck cancers such as esophageal cancer, head and neck cancer, nasopharyngeal carcinoma, oral cancer, oropharyngeal cancer, paranasal sinus and nasal cavity cancer, pharyngeal cancer, salivary gland cancer, and hypopharyngeal cancer; hematopoietic cancers such as acute biphenotypic leukemia, acute eosinophilic leukemia, acute lymphoblastic leukemia, acute myeloid leukemia, acute myeloid dendritic cell leukemia, AIDS-related lymphoma, anaplastic large cell lymphoma, angioimmunoblastic T-cell lymphoma, B-cell prolymphocytic leukemia, Burkitt's lymphoma, chronic lymphocytic leukemia, chronic myelogenous leukemia, cutaneous T-cell lymphoma, diffuse large B-cell lymphoma, follicular lymphoma, hairy cell leukemia, hepatosplenic T-cell lymphoma, Hodgkin's lymphoma, hairy cell leukemia, intravascular large B-cell lymphoma, large granular lymphocytic leukemia, lymphoplasmacytic lymphoma, lymphomatoid granulomatosis, mantle cell lymphoma, marginal zone B-cell lymphoma, Mast cell leukemia, mediastinal large B cell lymphoma, multiple myeloma/plasma cell neoplasm, myelodysplastic syndroms, mucosa-associated lymphoid tissue lymphoma, mycosis fungoides, nodal marginal zone B cell lymphoma, non-Hodgkin lymphoma, precursor B lymphoblastic leukemia, primary central nervous system lymphoma, primary cutaneous follicular lymphoma, primary cutaneous immunocytoma, primary effusion lymphoma, plasmablastic lymphoma, Sezary syndrome, splenic marginal zone lymphoma, and T-cell prolymphocytic leukemia; skin cancers such as basal cell carcinoma, squamous cell carcinoma, skin adnexal tumors (such as sebaceous carcinoma), melanoma, Merkel cell carcinoma, sarcomas of primary cutaneous origin (such as dermatofibrosarcoma protuberans), and lymphomas of primary cutaneous origin (such as mycosis fungoides); thoracic and respiratory cancers such as bronchial adenomas/carcinoids, small cell lung cancer, mesothelioma, non-small cell lung cancer, pleuropulmonary blastoma, laryngeal cancer, and thymoma or thymic carcinoma; HIV/AIDs-related cancers such as Kaposi sarcoma; epithelioid hemangioendothelioma; desmoplastic small round cell tumor; and liposarcoma.

In some aspects, the medical disorder can include a neurodegenerative disorder. Representative examples of neurodegenerative disorders that may be treated include, but are not limited to, Alzheimer's disease, Parkinson's disease, Huntington's disease and Huntington's disease-like syndromes, amyotrophic lateral sclerosis, progressive supranuclear palsy, corticobasal degeneration, chronic traumatic encephalopathy, multiple system atrophy, dementia with Lewy bodies, frontotemperal lobal degeneration, limbic-predominant age-related TDP-43 encephalopathy, spinocerebellar ataxias, dentatorubral-pallidoluysian atrophy, Creutzfeldt-Jakob disease, fatal familial insomnia, Alexander disease, Cerebral Autosomal Dominant Arteriopathy with Subcortical Infarcts and Leukoencephalopathy (CADASIL), neuronal intermediate filament inclusion disease, Riboflavin transporter deficiency neuronopathy.

In some aspects, the medical disorder can include an autoimmune disease. Representative examples of autoimmune disorders that may be treated include, but are not limited to: autoimmune disorders of the integumentary system (e.g., alopecia areata, autoimmune angioedema, autoimmune progesterone dermatitis, autoimmune urticaria, bullous pemphigoid, cicatricial pemphigoid, dermatitis herpetiformis, cermatomyositis, discoid lupus erythematosus, epidermolysis bullosa acquisita, erythema nodosum, gestational pemphigoid, hidradenitis suppurativa, lichen planus, lichen sclerosus, linear IgA disease, morphea, psoriasis, pemphigus vulgaris, scleroderma (systemic sclerosis), or vitiligo); autoimmune disorders of the digestive system (e.g., autoimmune enteropathy, autoimmune hepatitis, celiac disease, Crohn's disease, pernicious anemia, or ulcerative colitis); autoimmune disorders of the heart and vascular system (e.g., rheumatic heart disease, Kawasaki disease, giant cell arteritis, Takayasu's arteritis, Behget's disease, eosinophilic granulomatosis with polyangiitis (EGPA), granulomatosis with polyangiitis (GPA), IgA vasculitis (IgAV), leukocytoclastic vasculitis, lupus vasculitis, rheumatoid vasculitis, microscopic polyangiitis (MPA), polyarteritis nodosa (PAN), polymyalgia rheumatica, or urticarial vasculitis); autoimmune disorders of the urinary system (e.g., Goodpasture syndrome, IgA nephropathy, membranous nephropathy, lupus nephritis, interstitial nephritis, interstitial cystitis, or primary sclerosing cholangitis); autoimmune disorders of the nervous system (e.g., acute disseminated encephalomyelitis, acute motor axonal neuropathy, anti-NMDA receptor encephalitis, autoimmune encephalitis, Balo concentric sclerosis, Bickerstaff's encephalitis, chronic inflammatory demyelinating polyneuropathy, Guillain-Barre syndrome, Hashimoto's encephalopathy, Lambert-Eaton myasthenic syndrome, multiple sclerosis, myasthenia gravis, neuromyelitis optica (Devic's disease), restless legs syndrome, stiff-person syndrome, Sydenham's chorea, or transverse myelitis); autoimmune disorders of the endocrine system (e.g., Addison's disease, autoimmune oophoritis, autoimmune orchitis, autoimmune pancreatitis, autoimmune polyendocrine syndrome type 1 (APS1), autoimmune polyendocrine syndrome type 2 (APS2), autoimmune polyendocrine syndrome type 3 (APS3), diabetes mellitus type 1, endometriosis, Graves' disease, Hashimoto's thyroiditis, Ord's thyroiditis, or Sjögren syndrome); autoimmune disorders of the respiratory system (e.g., Goodpasture syndrome, eosinophilic granulomatosis with polyangiitis (EGPA), granulomatosis with polyangiitis (GPA), idiopathic pulmonary fibrosis, interstitial lung disease, pulmonary alveolar proteinosis, rheumatoid lung disease, or sarcoidosis); autoimmune disorders of the blood (e.g., autoimmune hemolytic anemia, immune thrombocytopenia, thrombotic thrombocytopenic purpura, antiphospholipid syndrome, or paroxysmal nocturnal hemoglobinuria); autoimmune disorders of the reproductive system (e.g., autoimmune orchitis, autoimmune oophoritis, endometriosis, or premature ovarian failure); autoimmune disorders of the eyes (e.g., autoimmune retinopathy, autoimmune uveitis, Cogan syndrome, Graves' ophthalmopathy, intermediate uveitis, ligneous conjunctivitis, Mooren's ulcer, neuromyelitis optica, opsoclonus myoclonus syndrome, optic neuritis, scleritis, Susac's syndrome, sympathetic ophthalmia, or Tolosa-Hunt syndrome); autoimmune disorders of the muscular system (e.g., dermatomyositis, fibromyalgia, inclusion body myositis, myositis, myasthenia gravis, neuromyotonia, paraneoplastic cerebellar degeneration, or polymyositis); and autoimmune comorbidities (e.g., chronic fatigue syndrome, complex regional pain syndrome, eosinophilic esophagitis, gastritis, POEMS syndrome, Raynaud's phenomenon, primary immunodeficiency, or pyoderma gangrenosum).

In some aspects, the medical disorder can include an infection disease, for example an infection with human immunodeficiency virus (HIV) or hepatitis B.

In some aspects, the medical disorder Can include a cardiovascular disease, for example, coronary artery disease, peripheral arterial disease, cerebrovascular disease (including stroke), renal artery stenosis, aortic aneurysm, cardiomyopathy, hypertensive heart disease, heart failure, pulmonary heart disease, cardiac dysrhythmias, endocarditis, myocarditis, eosinophilic myocarditis, valvular heart disease, congenital heart disease, or rheumatic heart disease.

Further representative medical disorders which may be treated via the methods described herein include, but are not limited to: diseases or disorders associated with the immune checkpoint or which may be treated via administration of an immune checkpoint inhibitor; diseases or disorders associated with platelet aggregation (e.g., thrombotic diseases such as myocardial infarction or ischemic stroke); multiple sclerosis; Non-Hodgkin's lymphoma; gastric cancer; Hodgkin's lymphoma; ovarian cancer; colorectal cancer; head and neck cancer; non-small cell lung cancer; solid tumors; breast cancer; lymphoblastic leukemia; hair cell leukemia; diffuse large B-cell lymphoma; follicular leukemia; chronic lymphocytic leukemia; B-cell lymphoma; triple negative breast cancer; metastatic urothelial cancer; multiple myeloma; melanoma; cervical cancer; systemic lupus erythematosus; metastatic uveal melanoma; hemophilia A; acute lymphoblastic leukemia; rheumatoid arthritis; Crohn's disease; high cholesterol; generalized myasthenia gravis; atopic dermatitis; ulcerative colitis; wet age-related macular degeneration; diabetic macular edema; psoriasis; and atopic dermatitis.

In some aspects, the compounds or compositions described herein may be administered in combination or alternation with one or more additional therapeutic agents as defined herein.

In some aspects, the present disclosure provides methods for degrading a target protein. In some aspects, the methods include contacting the target with any of the PROTACs of the present disclosure under conditions in which the PROTAC recruits the target protein for ubiquitination and degradation by a proteasome. In some aspects, the method is performed in vitro (e.g., in a tube, cell culture plate, cell, or the like) and finds use, e.g., in testing or research applications. In other aspects, the method is performed in vivo (e.g., in an individual to whom the PROTAC is administered) and finds use, e.g., in clinical or therapeutic applications, such as methods of treating a disease or disorder (e.g., cancer) in a subject in need thereof.

The active ingredient may be administered in such amounts, time, and route deemed necessary in order to achieve the desired result. The exact amount of the active ingredient will vary from subject to subject, depending on the species, age, and general condition of the subject, the severity of the medical disorder, the particular active ingredient, its mode of administration, its mode of activity, and the like. The active ingredient, whether the active compound itself, or the active compound in combination with an agent, is preferably formulated in dosage unit form for ease of administration and uniformity of dosage. It will be understood, however, that the total daily usage of the active ingredient will be decided by the attending physician within the scope of sound medical judgment. The specific therapeutically effective dose level for any particular subject will depend upon a variety of factors including the disorder being treated and the severity of the disorder; the activity of the active ingredient employed; the specific composition employed; the age, body weight, general health, sex and diet of the patient; the time of administration, route of administration, and rate of excretion of the specific active ingredient employed; the duration of the treatment; drugs used in combination or coincidental with the specific active ingredient employed; and like factors well known in the medical arts.

The active ingredient may be administered by any route. In some aspects, the active ingredient is administered via a variety of routes, including oral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, buccal, enteral, sublingual; by intratracheal instillation, bronchial instillation, and/or inhalation; and/or as an oral spray, nasal spray, and/or aerosol. In general, the most appropriate route of administration will depend upon a variety of factors including the nature of the active ingredient (e.g., its stability in the environment of the gastrointestinal tract), the condition of the subject (e.g., whether the subject is able to tolerate oral administration), etc.

The exact amount of an active ingredient required to achieve a therapeutically or prophylactically effective amount will vary from subject to subject, depending on species, age, and general condition of a subject, severity of the side effects or disorder, identity of the particular compound(s), mode of administration, and the like. The amount to be administered to, for example, a child or an adolescent can be determined by a medical practitioner or person skilled in the art and can be lower or the same as that administered to an adult.

Useful dosages of the active agents and pharmaceutical compositions disclosed herein can be determined by comparing their in vitro activity, and in vivo activity in animal models. Methods for the extrapolation of effective dosages in mice, and other animals, to humans are known to the art.

The dosage ranges for the administration of the compositions are those large enough to produce the desired effect in which the symptoms or disorder are affected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counterindications. Dosage can vary and can be administered in one or more dose administrations daily, for one or several days.

### Additional Aspects

1. A proteolysis targeting chimera (PROTAC) of Formula I:

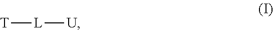

or a pharmaceutically acceptable salt or derivative thereof;
wherein:
T includes a target binding moiety capable of binding to a target protein;
L includes a linker moiety;
U includes a ubiquitin ligase-recruiting moiety capable of binding a ubiquitin ligase;
wherein any of T, L, or U further includes a first stimulus-reactive moiety Si, wherein S1 is reactive to a first stimulus; and
wherein any of T, L, or U further includes a second stimulus-reactive moiety S2, wherein S2 is reactive to a second stimulus, wherein the second stimulus is different than the first stimulus.

2. The PROTAC of any one aspect described herein, such as one of aspect 1, wherein upon exposure to only the first stimulus, only the second stimulus, or to neither of the first stimulus or second stimulus, the PROTAC of Formula I does not substantially recruit the target protein for ubiquitination and degradation by a proteasome.

3. The PROTAC of any one aspect described herein, such as any one of aspect 1 or aspect 2, wherein upon exposure to both the first stimulus and the second stimulus, the PROTAC of Formula I forms an active compound capable of recruiting the target protein for ubiquitination and degradation by a proteasome.

4. The PROTAC of any one aspect described herein, such as any one of aspects 1-3, wherein the first stimulus, the second stimulus, or both independently include one or more stimuli present within a cell.

5. The PROTAC of any one aspect described herein, such as any one of aspects 1-4, wherein the first stimulus, the second stimulus, or both independently include one or more stimuli delivered exogenously to a cell.

6. The PROTAC of any one aspect described herein, such as any one of aspects 1-5, wherein the first stimulus, the second stimulus, or both independently include one or more stimuli associated with a disease.

7. The PROTAC of any one aspect described herein, such as any one of aspects 1-6, wherein the first stimulus, the second stimulus, or both independently include hypoxia, reactive oxygen species, glutathione, one or more enzymes, light, or radiation.

8. The PROTAC of any one aspect described herein, such as any one of aspects 1-7, wherein L includes the first stimulus-reactive moiety Si, the second stimulus-reactive moiety S2, or both.

9. The PROTAC of any one aspect described herein 1, such as any one of aspects 1-8, wherein S¹ or S² is

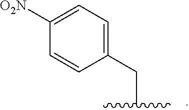 [SMILES: *Cc1ccc([N+](=O)[O-])cc1]

10. The PROTAC of any one aspect described herein, such as any one of aspects 1-9, wherein S¹ or S²

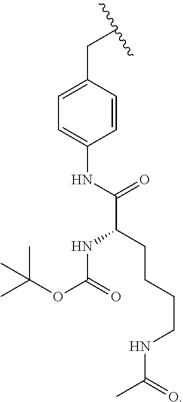 [SMILES: *Cc1ccc(NC(=O)[C@H](CCCCNC(C)=O)NC(=O)OC(C)(C)C)cc1]

11. The PROTAC of any one aspect described herein, such as any one of aspects 1-10, wherein the linker moiety is selected from L1

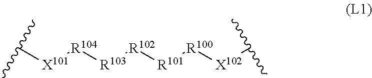

wherein:
X¹⁰¹ and X¹⁰² are independently at each occurrence selected from a bond, 6- to 10-membered monocyclic or bicyclic aryl, 5- to 10-membered monocyclic or bicyclic heteroaryl, C₃-C₇ cycloalkyl, 3- to 8-membered monocyclic or bicyclic heterocycle, C₁-C₂₀ alkyl, C₂-C₂₀ alkenyl, C₂-C₂₀ alkynyl, NR¹⁰⁵, O, C(O), and S;
R¹⁰⁰, R¹⁰¹, R¹⁰², R¹⁰³, and R¹⁰⁴ are independently at each occurrence selected from the group consisting of a bond, —C(O)—, —C(O)O—, —OC(O)—, —SO₂—, —S(O)—, C(S)—, —C(O)NR¹⁰⁵—, —NR¹⁰⁵C(O)—, —O—, —S—, —NR¹⁰⁵—, —P(O)(OR¹⁰⁵))—, C₁-C₂₀ alkyl, C₂-C₂₀ alkenyl, C₂-C₂₀ alkynyl, 6- to 10-membered monocyclic or bicyclic aryl, 3- to 8-membered monocyclic or bicyclic heterocycle, C₃-C₇ cycloalkyl, 5- to 10-membered monocyclic or bicyclic heteroaryl, lactic acid, or glycolic acid, each of which may be optionally substituted with one or more (for example, 1, 2, 3, or 4) substituents independently selected from R¹⁴⁰;
R¹⁰⁵ independently selected at each occurrence from R¹²⁰, R¹¹⁰C(O)—, R¹¹⁰C(N)—, R¹¹⁰S(O)—, and R¹¹⁰S(O)₂—, each of which may be optionally substituted with one or more R¹⁴⁰ groups as allowed by valency;
R¹¹⁰ is independently selected at each occurrence from hydrogen, halo, C₁-C₆alkyl, C₁-C₆haloalkyl, C₂-C₆alkenyl, C₂-C₆alkynyl, (C₃-C₇ cycloalkyl)-(C₀-C₆ alkyl)-, (4- to 6-membered heterocycle)-(C₀-C₆ alkyl)-, (5- to 10-membered monocyclic or bicyclic aryl)-(C₀-C₆ alkyl)-, (5- to 10-membered monocyclic or bicyclic heteroaryl)-(C₀-C₆ alkyl)-, —OR¹²⁰, —SR¹²⁰, and —NR¹²⁰R¹³⁰, each of which may be optionally substituted with one or more R¹⁴⁰ groups as allowed by valency;
R¹²⁰ and R¹³⁰ are independently selected at each occurrence from hydrogen, C₁-C₆alkyl, C₁-C₆haloalkyl, C₂-C₆alkenyl, C₂-C₆alkynyl, (C₃-C₇ cycloalkyl)-(C₀-C₆ alkyl)-, (4- to 6-membered heterocycle)-(C₀-C₆ alkyl)-, (5- to 10-membered monocyclic or bicyclic aryl)-(C₀-C₆ alkyl)-, (5- to 10-membered monocyclic or bicyclic heteroaryl)-(C₀-C₆ alkyl)-, each of which may be optionally substituted by one or more R¹⁴⁰ groups as allowed by valency; and
R¹⁴⁰ is independently at each occurrence selected from the group consisting of hydrogen, halo, nitro, cyano, azido, oxo, C₁-C₆ alkyl, C₁-C₆ haloalkyl, C₂-C₆ alkenyl, C₂-C₆ alkynyl, (C₃-C₇ cycloalkyl)(C₀-C₆ alkyl)-, (3- to 8-membered monocyclic or bicyclic heterocycle)-(C₀-C₆ alkyl)-, (6- to 10-membered monocyclic or bicyclic aryl)-(C₀-C₆ alkyl)-, (5- to 10-membered monocyclic or bicyclic heteroaryl)-(C₀-C₆ alkyl)-, R¹⁴¹O—, R¹⁴¹S—, R¹⁴¹R¹⁴²N—, R¹⁴³C(O)—, R¹⁴³C(O)—O—, R¹⁴²C(O)—NR¹⁴²—, R¹⁴³S(O)₂—, R¹⁴³S(O)₂—O—, and R¹⁴³S(O)₂—NR¹⁴²—, wherein R¹⁴³ is independently selected at each occurrence from R¹⁴¹, halo, R¹⁴¹O—, and R¹⁴¹R¹⁴²N—, and wherein R¹⁴¹ and R¹⁴² are independently selected at each occurrence from hydrogen, C₁-C₆ alkyl, C₁-C₆ haloalkyl, C₂-C₆ alkenyl, C₂-C₆ alkynyl, (C₃-C₇ cycloalkyl)(C₀-C₆ alkyl)-, (3- to 8-membered monocyclic or bicyclic heterocycle)-(C₀-C₆ alkyl)-, (6- to 10-membered monocyclic or bicyclic aryl)-(C₀-C₆ alkyl)-, and (5- to 10-membered monocyclic or bicyclic heteroaryl)-(C₀-C₆ alkyl)-;
wherein each of X¹⁰¹, X¹⁰², R¹⁰⁰, R¹⁰¹, R¹⁰², R¹⁰³, and R¹⁰⁴ may be optionally substituted by S¹ or S¹.

12. The PROTAC of any one aspect described herein, such as any one of aspects 1-11, wherein L is selected from

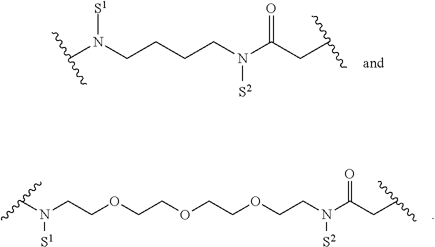

13. The PROTAC of any one aspect described herein, such as any one of aspects 1-12, wherein the target binding moiety includes or is derived from a ligand of the target protein.

14. The PROTAC of any one aspect described herein, such as any one of aspects 1-13, wherein the target binding moiety includes or is derived from a ligand for BRD4 or CDK9.

15. The PROTAC of any one aspect described herein, such as any one of aspects 1-14, wherein the target binding moiety is selected from

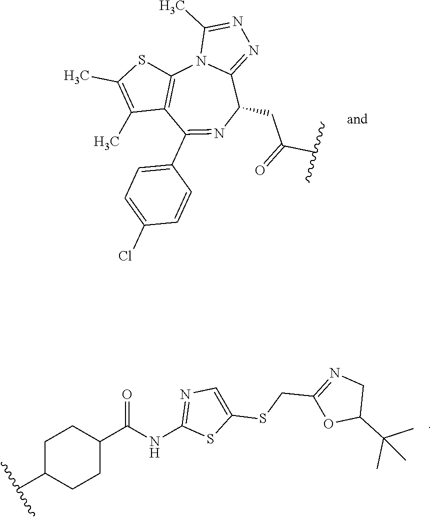 [SMILES: *C(=O)C[C@@H]1N=C(c2ccc(Cl)cc2)c2c(sc(C)c2C)-n2c(C)nnc21 ; *C1CCC(C(=O)Nc2ncc(SCC3=NCC(C(C)(C)C)O3)s2)CC1]

16. The PROTAC of any one aspect described herein, such as any one of aspects 1-15, wherein the ubiquitin ligase-recruiting moiety is capable of binding to cereblon.

17. The PROTAC of any one aspect described herein, such as any one of aspects 1-16, wherein the ubiquitin ligase-recruiting moiety is selected from

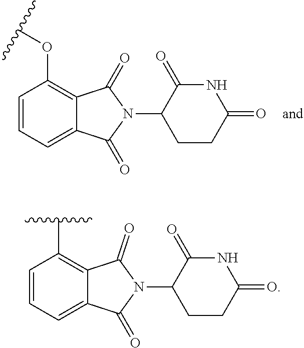 [SMILES: *Oc1cccc2c1C(=O)N(C1CCC(=O)NC1=O)C2=O ; *c1cccc2c1C(=O)N(C1CCC(=O)NC1=O)C2=O]

18. A pharmaceutical composition comprising a PROTAC of any one aspect described herein, such as any one of aspects 1-17, or a pharmaceutically acceptable salt or derivative thereof, and a pharmaceutically acceptable carrier or excipient.

19. A method of treating a medical disorder that is mediated by a target protein in a subject in need thereof, the method comprising administering a therapeutically effective amount of a PROTAC of any one aspect described herein, such as any one of aspects 1-17, or a pharmaceutically acceptable salt or derivative thereof, or any pharmaceutical composition described herein, such as one of aspect 18, wherein the target protein is degraded or downregulated by the PROTAC.

20. The method of any one aspect described herein, such as one of aspect 17, wherein the medical disorder is a cancer, a neurodegenerative disorder, or an autoimmune disorder.

A number of aspects of the disclosure have been described. Nevertheless, it will be understood that various modifications may be made without departing from the spirit and scope of the disclosure. Accordingly, other aspects are within the scope of the following claims.

By way of non-limiting illustration, examples of certain aspects of the present disclosure are given below.

### EXAMPLES

The following examples are set forth below to illustrate the compounds, compositions, articles, devices, and methods claimed herein, along with associated methods and results according to the disclosed subject matter. These examples are not intended to be inclusive of all aspects of the subject matter disclosed herein, but rather to illustrate representative methods and results. These examples are not intended to exclude equivalents and variations of the present disclosure, which are apparent to one skilled in the art.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures, and other reaction ranges and conditions that can be used to optimize the product purity and yield obtained from the described process. Only reasonable and routine experimentation will be required to optimize such process conditions.

### Dual-Action-Only PROTACS

Proteolysis targeting chimera (PROTAC) based degraders have attracted a great amount of attention because of their high potency, often attributed to its pseudocatalytic potential. It remains then that off-target tissue homing of these potent drugs could also have undesirable consequences. We seek to design and develop a generalizable AND-logic gated PROTAC, where a molecule would become an active protein degrader only in the concurrent presence of two different disease-relevant endogenous stimuli. We test this Dual-Action-Only PROTAC (DAO-PROTAC) concept with hypoxia and cathepsin L as the orthogonal stimuli. We design and demonstrate that dormant DAO-dBET1 can be activated only in the presence of both hypoxia and Cath-L to generate the active dBET1 and degrade the transcriptional coactivator BRD4. We also show that the dormancy of DAO-dBET1 translates to considerable mitigation of cytotoxicity. This example demonstrates the potential advantages of a DAO-PROTAC over the corresponding free PROTAC and single-stimulus triggerable PROTACs.

Targeted protein degraders, such as proteolysis targeting chimeras (PROTACs) and molecular glues, have garnered increasing attention because of its pseudo-catalytic mode of action that can result in higher efficacy and its potential to alleviate resistance development.¹⁻³ Additionally, compared to occupancy-based drugs that temporally inhibit a protein function, these molecules offer a generalizable strategy to entirely degrade a protein of interest (POI). This increased potency also highlights the need for cell specificity.^{4,5} Because degradation of POIs leads to deletion of all essential functions of that protein, their off-target accumulation could prove even more deleterious. An attractive approach to tackle this challenge is to mute these degraders and then activate them on location using endogenous, tissue-specific stimuli. In this example, we disclose a generalizable approach for activatable PROTACs using two disease-relevant stimuli.

There are few reports on activatable PROTACs that have focused on a single stimulus.⁵⁻¹⁰ We posit that leveraging Boolean logic in designing a discriminatory PROTAC that necessitates the concurrent presence of two orthogonal stimuli that are overexpressed in pathological cells, compared to healthy cells, would substantially increase target specificity and lower undesirable off-target toxicity. This expectation is based on the reasonable assumption that while one of the two overexpressed factors might be present with some activity at another off-target location, the likelihood that two orthogonal disease-specific stimuli would be concurrently present at the same off-target location becomes substantially lower.¹¹

For Dual-Action-Only PROTAC (DAO-PROTAC) design, we chose hypoxia and cathepsin L (Cath-L) as the stimuli because of their relevance in oncology. Hypoxic areas develop in 90% of solid tumors, attributed to increased metabolic oxygen demands of their high proliferative and growth rates, characterized by increased hypoxia inducible factors (HIFs) and oxidoreductase enzyme levels.¹² Similarly, elevated activity of endogenous cysteine protease Cath-L has also been shown to play crucial roles in tumor development and disease progression.^{13,14}

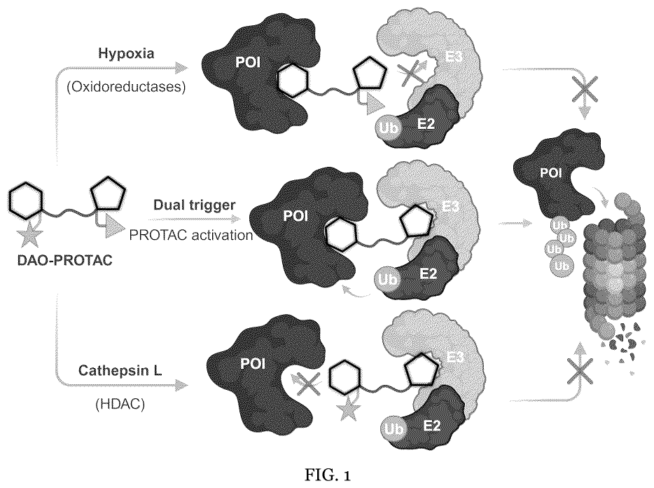

The heterobivalent nature of PROTACs conveniently lends itself to our AND-gate strategy. In this design, the ligand for the POI and the ligand for the E3 ligase are masked by functionalities responsive to hypoxia and Cath-L respectively. When the hypoxia-induced unmasking of the POI-ligand occurs, the resultant molecule would still not be able to engage the E3 ligase. Similarly, when Cath-L unmasks the E3 ligase-ligand, the product does not engage the POI. It is only when the DAO-PROTAC encounters both conditions, the resulting functional PROTAC would induce degradation (FIG. 1).

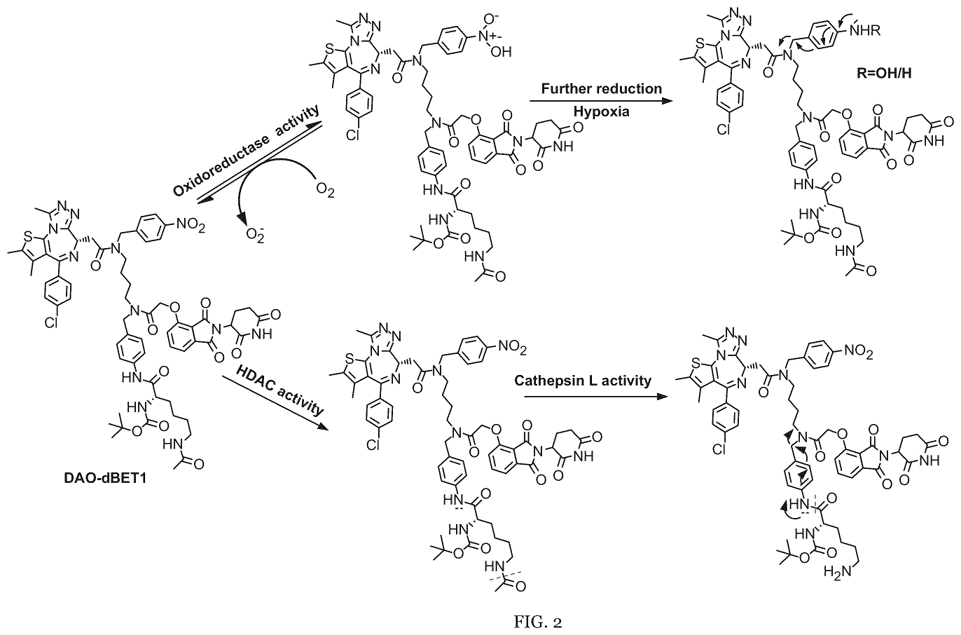

dBET1, a PROTAC targeting the transcriptional coactivator BRD4, is used to demonstrate the DAO principle in this example (FIG. 2). BRD4, a BET protein, is implicated as a promoter of tumor occurrence, development, and metastasis. dBET1 consists of a BET-inhibitor, JQ1, and an E3 ligase cereblon (CRBN) binding ligand, thalidomide.¹⁵

We chose dBET1 as the model to create DAO-dBET1, because: (i) it presents two amide moieties proximal to both ligands, which present convenient handles to introduce the masking functionalities at the JQ1 and the thalidomide sides; (ii) introducing the steric bumps at the linker, rather than on the ligand, offers a more generalizable strategy for other heterobivalent ligand combinations; and (iii) the masking step converts secondary amides in the linker to tertiary amides that are substantially more stable for hydrolysis—note that hydrolytic stability of the linker is crucial for the oral translation of PROTACs.¹⁶ Overall, the findings of this example would have implications beyond the BRD4 degradation targeted here.

Structure of DAO-dBET1 and its stimuli-induced conversion to dBET1 are shown in FIG. 2. The hypoxia activatable moiety was introduced by simply incorporating 4-nitrobenzyl group on the JQ1 side of dBET1 to convert that to a tertiary amide. As the 4-nitrobenzyl functionality encounters intracellular oxidoreductases under low oxygen concentration, stepwise single electron transfers take place leading to the initial formation of an arylhydroxylamine followed by the formation of an arylamine (FIG. 2).¹⁷ The increased electron-donating character of either of these functionalities would ultimately result in self-immolative uncaging to form the secondary amide. Similarly, an ε-acetylated N-Boc-lysine moiety was introduced on the thalidomide side for Cath-L-based unmasking.^{13,18} This mask was also introduced as an alkyl group with an arylmethylene functionality at the carboxy-terminus of the molecule. As Cath-L cleaves the amide moiety at the C-terminus of the N-Boc-lysine, a similar self-immolative cascade reaction would liberate the secondary amide at the thalidomide terminus (FIG. 2). Note that ε-acetyl-N-Boc-lysine is converted to the Cath-L substrate, N-Boc-lysine, by histone deacetylase (HDAC). This modification was installed because extracellular cathepsins cannot cleave the C-terminus of ε-acetyl-N-Boc-lysine but can cleave N-Boc-lysine.¹³ By imposing the initial processing of ε-acetyl-N-Boc-lysine with HDACs, an intracellular enzyme, we envisaged that the activation of the thalidomide end of DAO-dBET1 would be more specific to intracellular Cath-L.

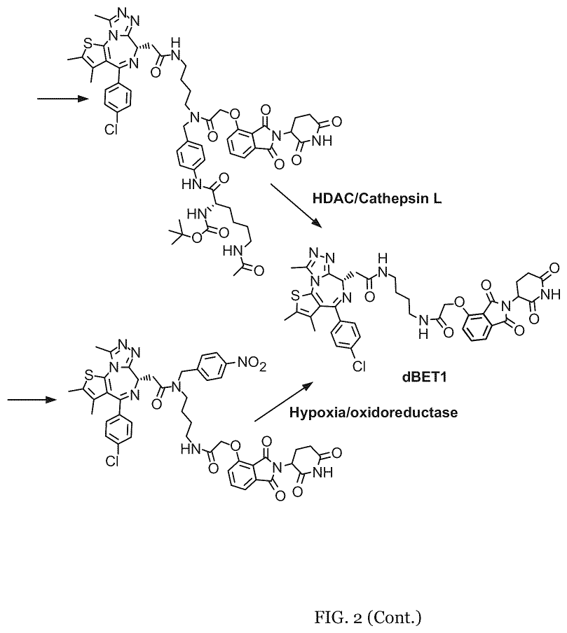

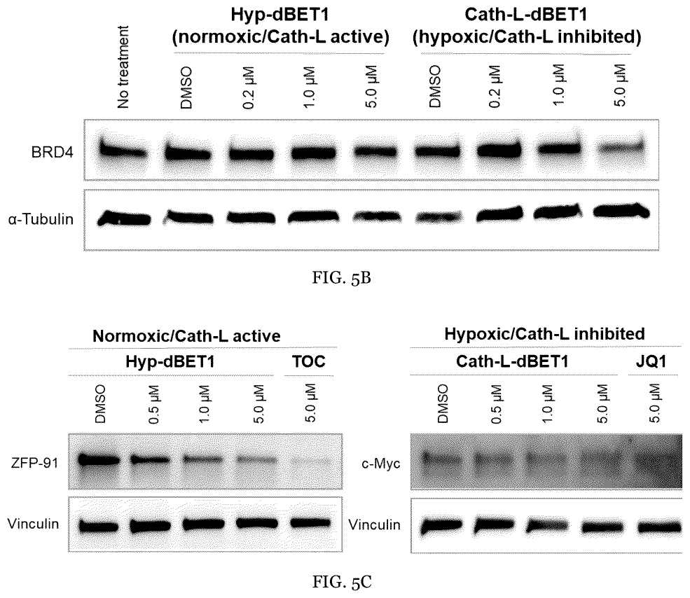

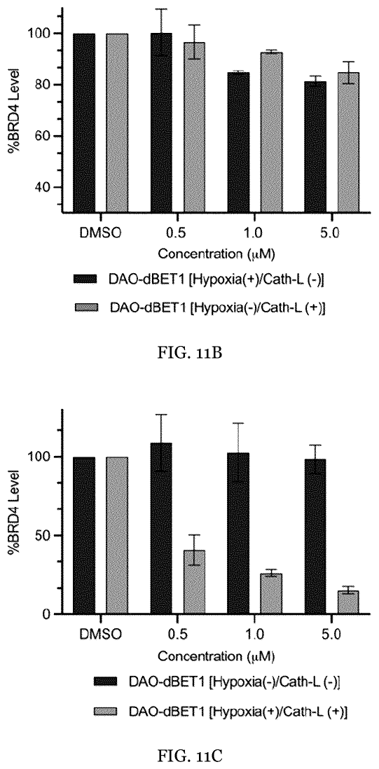

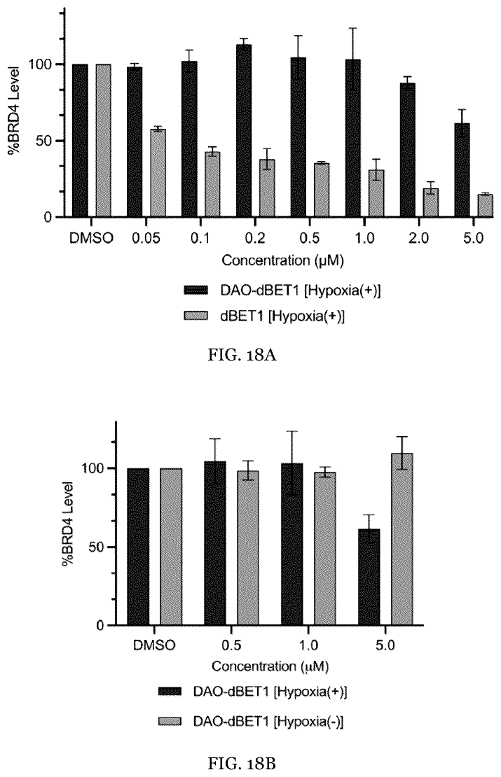

To test the design hypothesis, we first investigated the activity of the DAO-dBET1 in MDA-MB-231 cells (FIG. 3A). These cancer cells have inherently high levels of Cath-L expression (FIG. 17). However, because hypoxia is a feature in solid tumors and is not carried over at a cellular level, we induced hypoxia using cobalt chloride (CoCl₂) for the in vitro experiments. CoCl₂ has been reported to block the proteasomal degradation of HIF-1α and reduce cellular oxygen levels (FIG. 17).¹⁹ DAO-dBET1 exhibits a commendable degradation of BRD4 with a DC50 of 250 nM. We were gratified to find this to be comparable to the unmodified dBET1 (DC50 150 nM) (FIG. 8). To test the possibility of protein degradation in the absence of both triggers, we used Z-Phe-Tyr-CHO (Z-FY-CHO)^{13,18} inhibitor of Cath-L without the hypoxia-inducing CoCl₂. In this case, we observe little to no degradation of BRD4 (FIG. 3B). In cell viability assays, DAO-dBET1 exhibited an IC50 of 281 nM in the presence of both hypoxia and Cath-L triggers, very similar to that of unmodified dBET1 (167 nM) (FIG. 3C). In absence of both the triggers, however, there was no observable cytotoxicity even at 10 μM concentration. We further checked cell apoptosis using annexin V and propidium iodide (PI) staining assays, post 72 h treatment. In the presence of both triggers, apoptosis rates were significantly higher for dBET1 and DAO-dBET1 treatment (59% and 52% respectively at 1 μM concentrations), relative to the control DMSO (25%) (FIG. 3D). On the other hand, in absence of both stimuli, DAO-dBET1 treatment resulted in only ˜9% increase in apoptosis compared to the control. Caspase glo assay further supports the results from the apoptosis assay. While the luminescence from caspase 3/7 activation increased by 1.67 and 1.42 folds for dBET1 and DAO-dBET1 incubations respectively, this enhancement was negligible for the DAO dBET1 treatment in absence of any trigger (FIG. 22). These results corroborate our hypothesis of requiring AND-gated stimuli for the in-cell activation of DAO-dBET1.

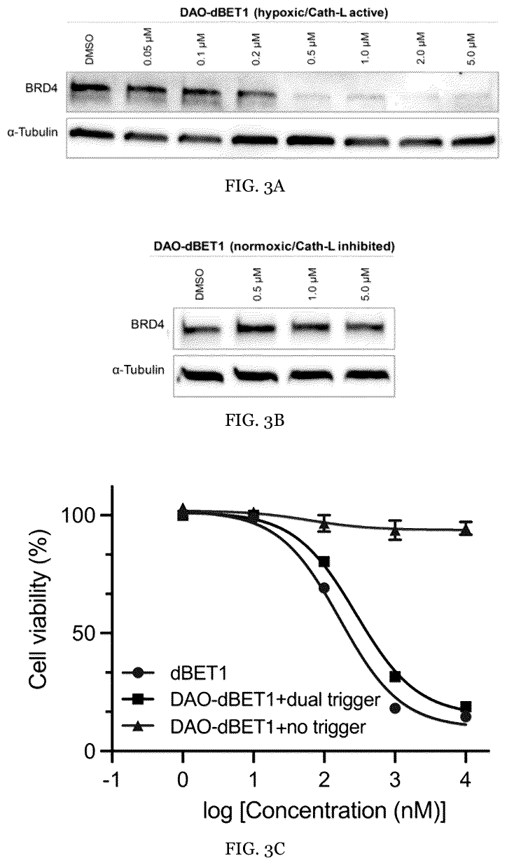

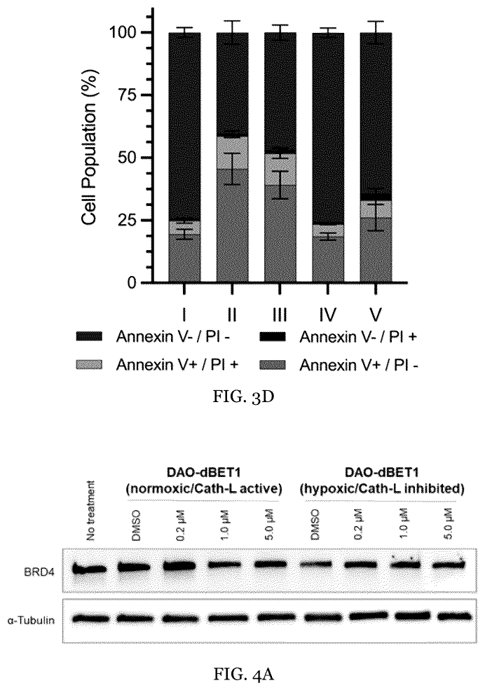

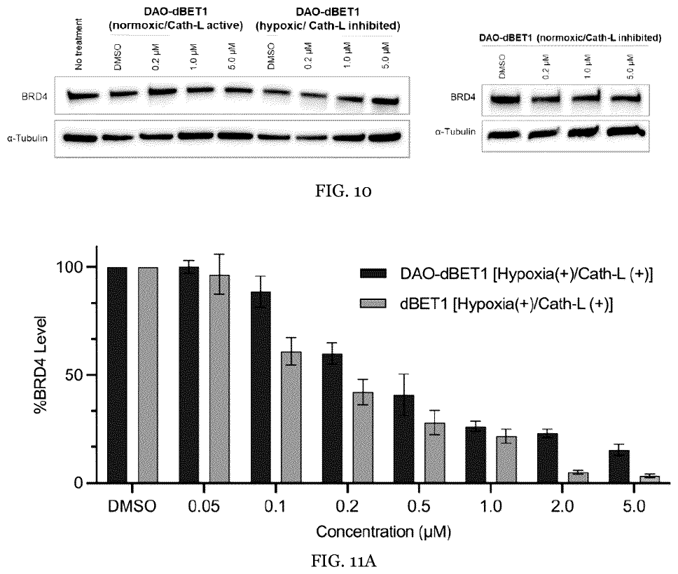

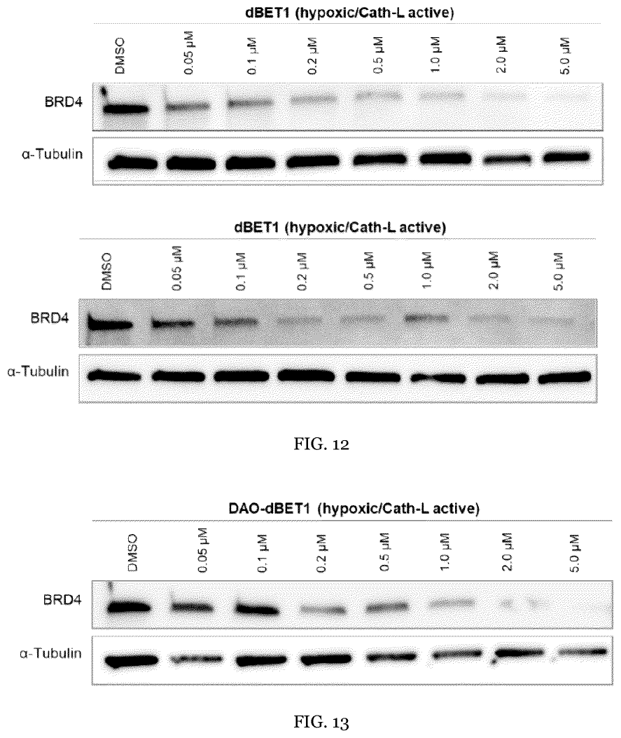

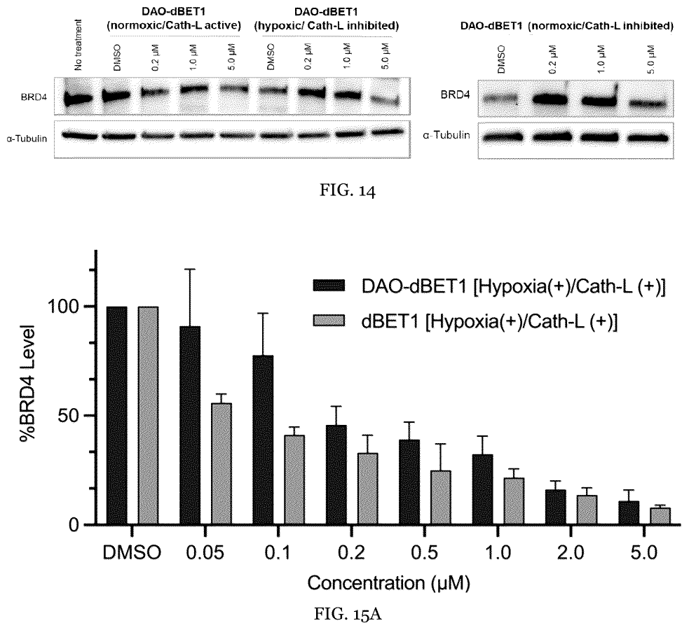

Interestingly, in presence of just one of the stimuli, BRD4 degradation by DAO-dBET1 is negligible (FIG. 4A), yet it shows significant cell killing after 72 h treatment (FIG. 4B). We observed a 33% and 31% higher apoptosis rate for DAO-dBET1 treatments in hypoxia-only and Cath-L-only treatments respectively, compared to the controls (FIG. 4C). The induced cell death is also observed with the increases in caspase 3/7 activity by 1.40 and 1.25 folds respectively (FIG. 4D). In investigating the reason for the observed toxicity for DAO-dBET1 in the presence of one of the triggers, we hypothesized that that either of the liberated ligands could act as an effector. The uncaged JQ1 or thalidomide can regulate BRD4 or CRBN function respectively. To further investigate this, we independently synthesized the mono-liberated products of DAO-dBET1, viz., Hyp-dBET1 and Cath-L-dBET1 (FIG. 5A). In the presence of their respective triggers, both Hyp-dBET1 and Cath-L-dBET1 exhibit DC50 and IC50 values comparable to that of dBET1 (FIGS. 16, 18A-18B). As anticipated, in their absence, i.e., Hyp-dBET1 in normoxic/Cath-L active and Cath-L-dBET1 in hypoxic/Cath-L inhibited, no BRD4 degradation is observed (FIG. 5B). Nonetheless, their toxicities in MDA-MB-231 cells are significant and comparable to thalidomide and JQ1 respectively (FIGS. 18A-18B, 19). To further test the role of target protein regulation by the free ligands, we immunoblotted the downstream proteins of CRBN and BRD4, viz., ZFP-91 (zinc finger protein) and c-Myc that are reported to be downregulated by thalidomide and JQ1 treatments respectively.^{20,21} Concentration-dependent depletions of c-Myc and carbonic anhydrase 9 were observed with Cath-L-dBET1 and similarly of ZFP-91 with Hyp-dBET1 in the absence of either trigger, at levels comparable to that of JQ1 and thalidomide treatments respectively (FIGS. 5C, 19). However, the expressions of c-Myc and ZFP-91 levels were unaltered when DAO-dBET1 was treated in absence of any stimuli (FIG. 5D). Together, these results highlight the enhanced safety of the doubly caged heterobivalent molecule, compared to the free drug or even the mono-caged prodrug.

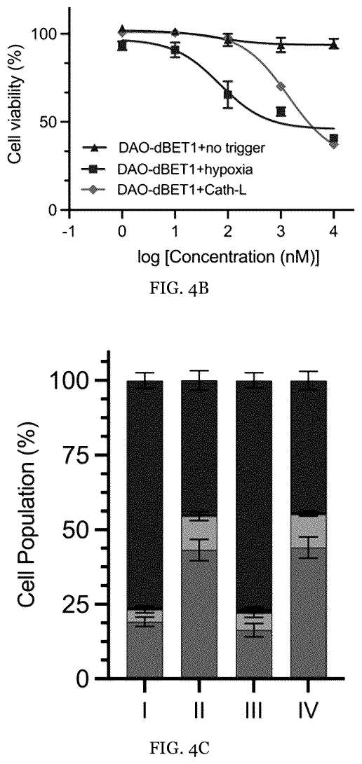

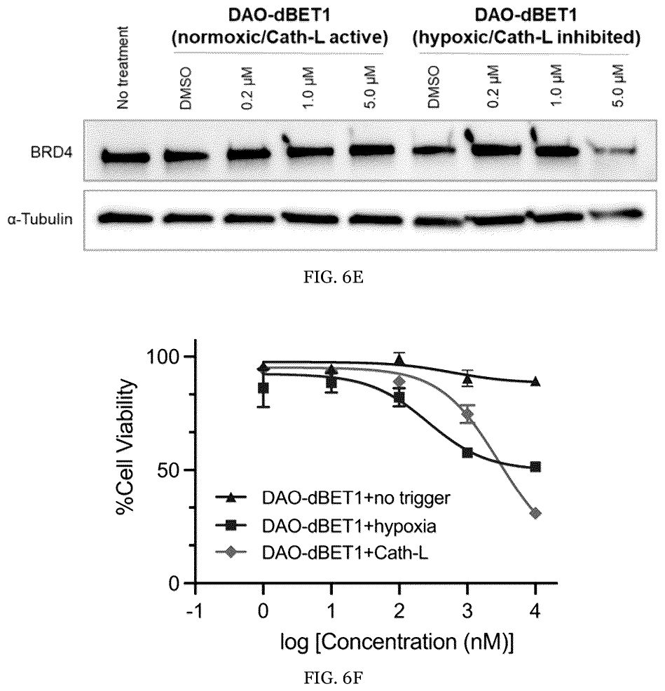

To investigate if DAO-dBET1 performs similarly in another cancer cell line, we chose HeLa cells that also over overexpress Cath-L and exhibits enhanced HIF-1α expression in the presence of CoCl₂ (FIG. 17). DAO-dBET1 exhibits a DC50 of 180 nM in the presence of both triggers, comparable to that of dBET1 (100 nM), but remains dormant in the absence of both triggers (FIGS. 6A-6B, 11A-11C). Likewise, while DAO-dBET1 is muted in the absence of the triggers, it was still toxic in presence of both of the stimuli, as indicated by the cell viability and apoptosis (FIGS. 4C-4D). Subsequently, we also verified cytotoxicity of the mono-liberated products of DAO-dBET1 using cell viability and apoptosis assays. In presence of either hypoxia or Cath-L activity only, while there was no observable BRD4 protein degradation, DAO-dBET1 showed substantial cell death with apoptosis rates of 41% and 32% respectively in the annexin V staining, indicating that unmasked JQ1 and thalidomide can regulate BRD4 and CRBN functions in HeLa cells also.

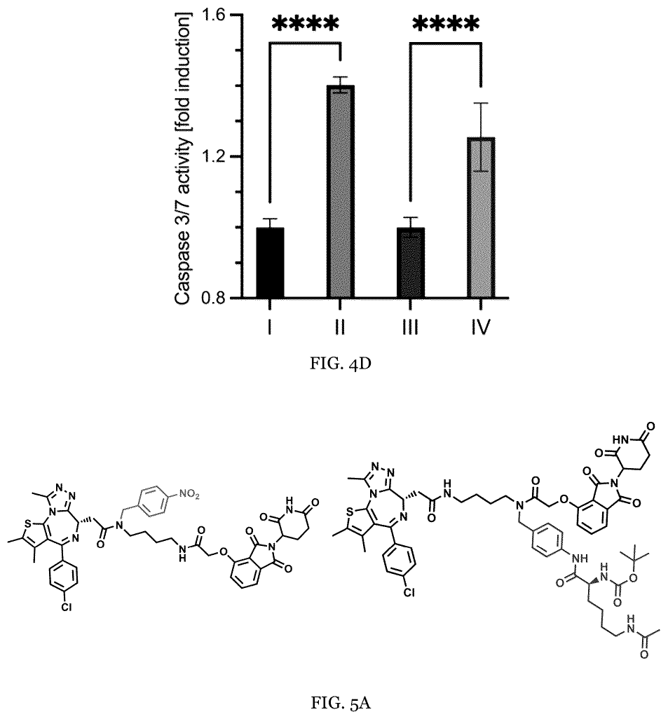

As an additional control, we used a healthy kidney cell line, HEK-293, that exhibits low levels of Cath-L (FIG. 17). The expression of HIF-1α in these cells can still be enhanced with CoCl₂ treatment (FIG. 17). Therefore, even in the presence of 200 μM CoCl₂ and the absence of Cath-L inhibitor Z-FY-CHO, DAO-dBET1 treatment does not cause significant BRD4 degradation even at high concentrations (FIGS. 7A-7B). However, the cell kill remains comparable in the presence of CoCl₂, regardless of the presence of Z-FY-CHO. This is attributed to the JQ1 uncaging due to the hypoxic conditions, while thalidomide remaining protected as a result of low Cath-L expression in HEK-293 cells (FIG. 7C). Caspase glo assay further indicates that the observed cytotoxicity is due to the mono-deprotection by the hypoxia trigger (FIG. 7D). Finally, DAO-dBET1 treatment under hypoxic conditions resulted in downregulation of c-Myc, while not causing any significant change in ZFP-91 expression even in absence of the Z-FY-CHO inhibitor due to the inherently low expression levels of Cath-L (FIGS. 7E-7F). Put together, the DAO-dBET1 is substantially safe in the healthy HEK-293 cells.

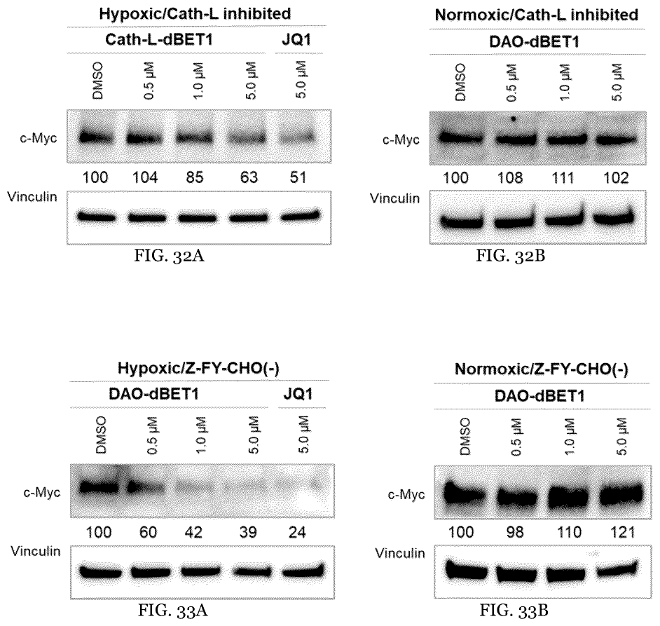

To further evaluate the generalizability of the dual-caging strategy, we designed a second DAO-PROTAC, DAO-THAL-SNS-032, which targets cyclin-dependent kinase 9 (CDK9) via the CRBN E3 ligase.²² CDK9 regulates RNA polymerase II activity and modulates the expression of genes implicated in cancer proliferation and metastasis. In contrast to DAO-dBET1, DAO-THAL-SNS-032 incorporates swapped stimuli-responsive moieties: a hypoxia-sensitive group on the thalidomide end and a Cathepsin L (Cath-L)-cleavable unit on the SNS-032 end. Additionally, the 4-nitrobenzyl group was replaced with a 4-nitrobenzylcarbamate to enhance stability and responsiveness (FIG. 34).

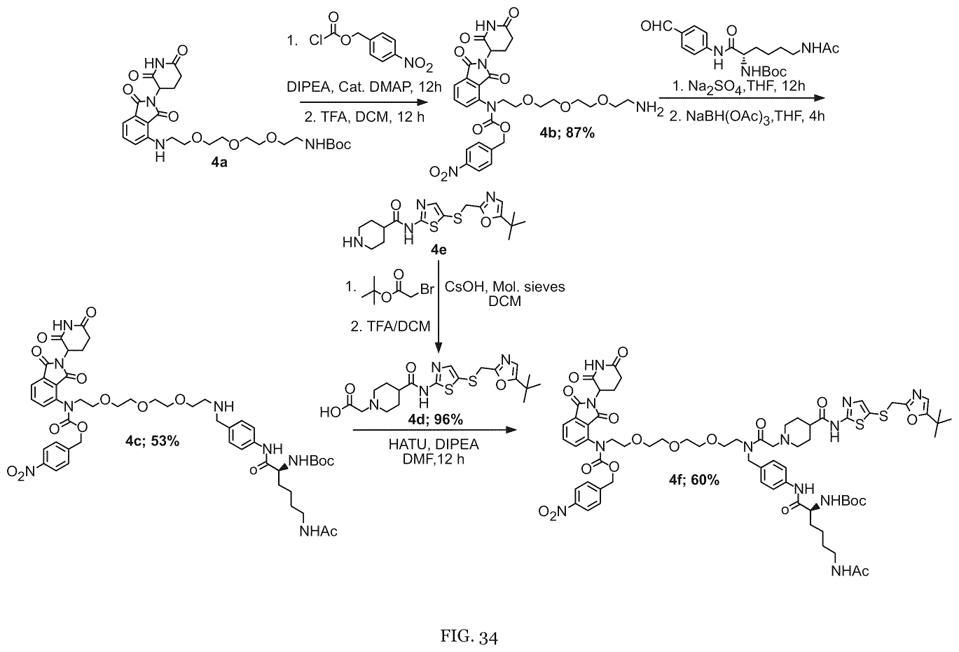

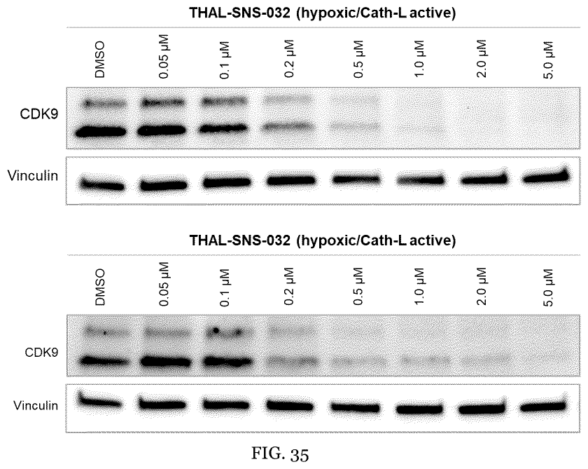

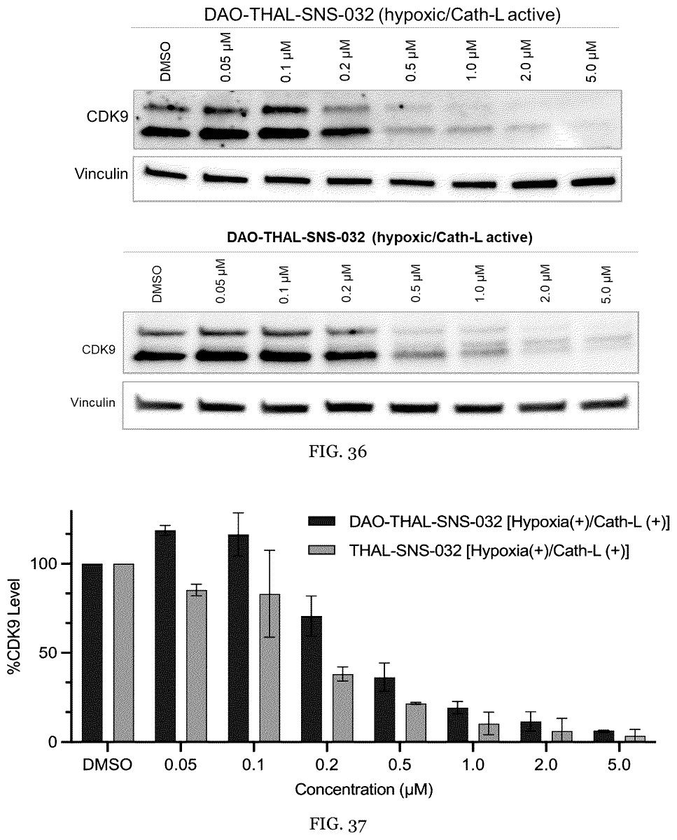

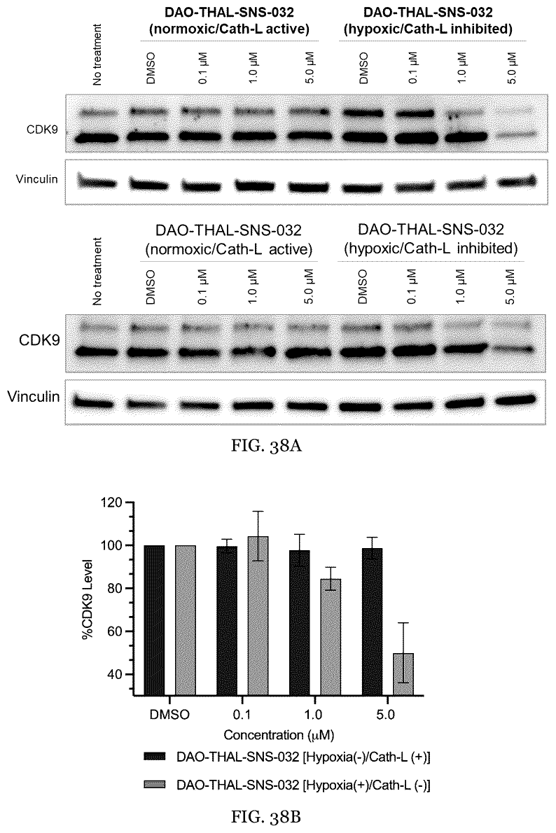

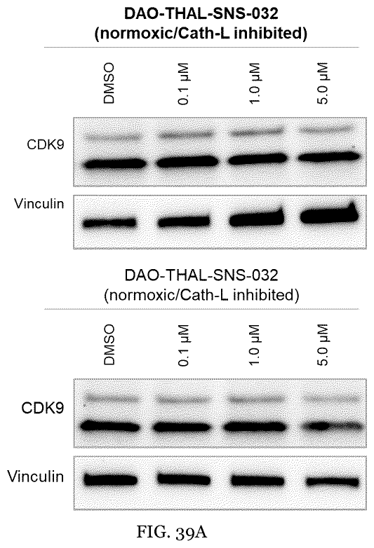

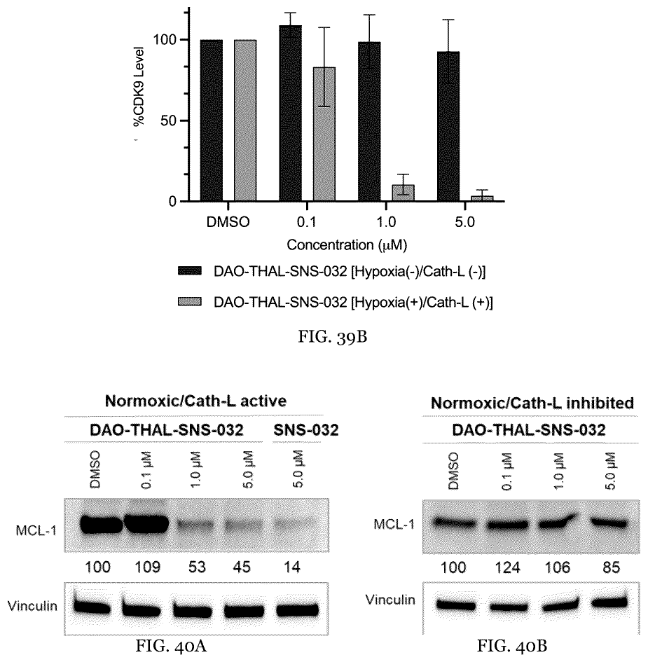

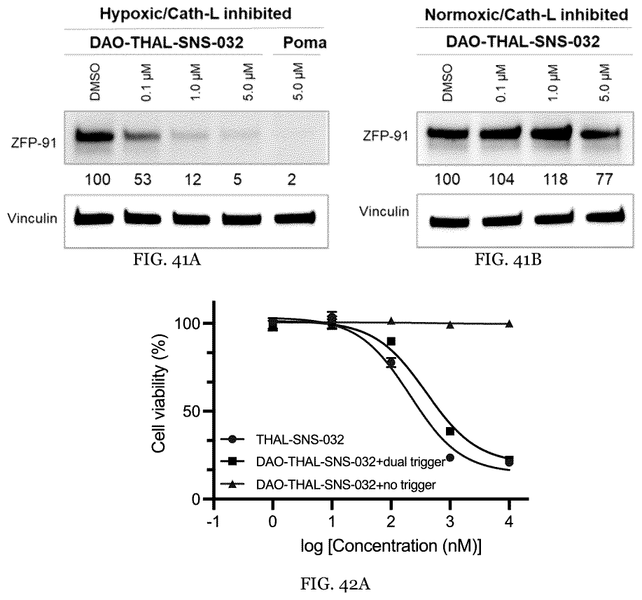

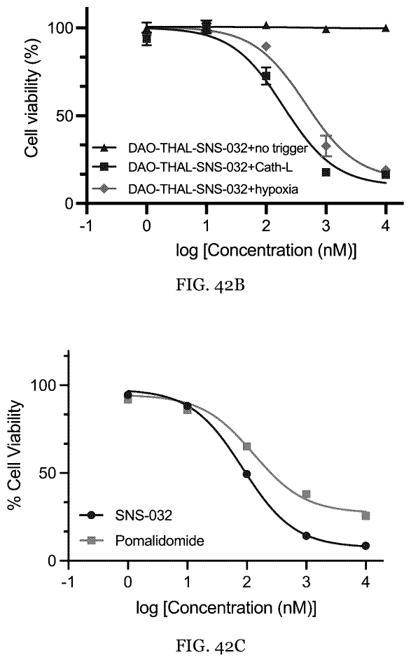

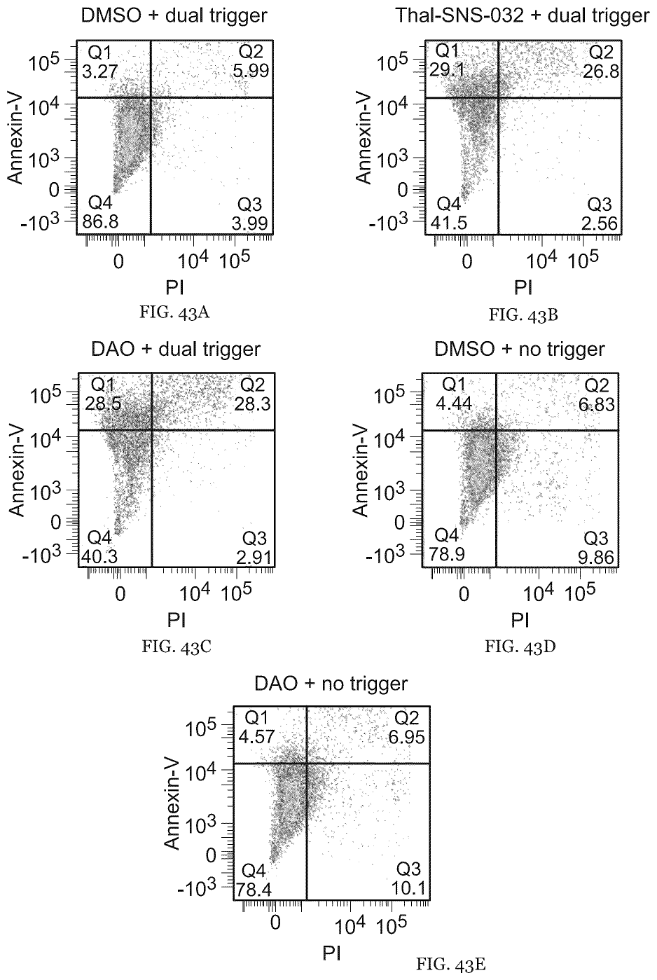

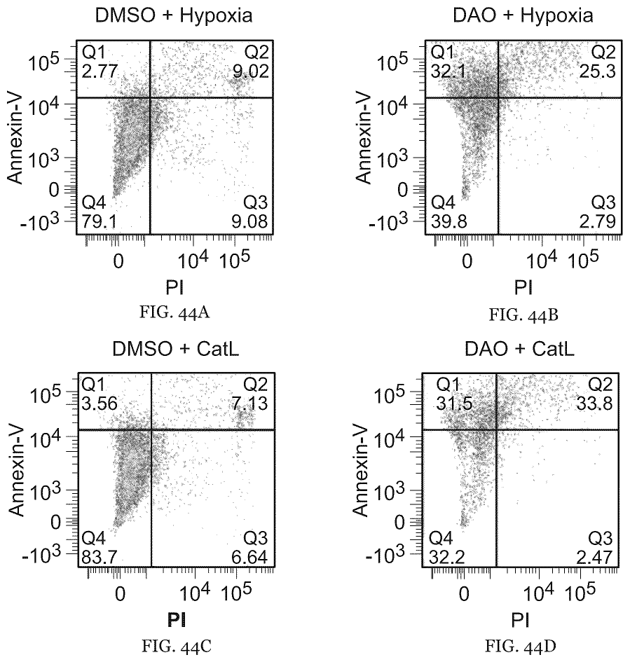

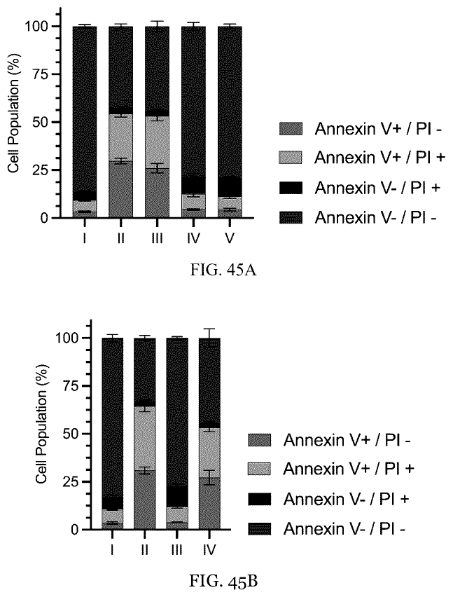

Under hypoxic conditions, DAO-THAL-SNS-032 efficiently degraded CDK9 (DC₅₀=265 nM), closely matching the activity of the uncaged parent compound THAL-SNS-032 (DC₅₀=172 nM) (FIGS. 35-37), while remaining inactive in the absence of both stimuli (FIGS. 39A-39B). The compound's cytotoxic potency (IC50) was likewise preserved (FIG. 42A). Though largely inert at high concentrations without activation, DAO-THAL-SNS-032 exhibited potent cytotoxicity upon uncaging triggered by either hypoxia or Cath-L (FIGS. 38A-38B, 42B), consistent with the effects of the individual ligands (FIG. 42C). These observations were further corroborated by annexin V/PI apoptosis assays (FIGS. 43A-43E, 44A-44D, 45A-45B) and the downregulation of downstream targets, including MCL-1 and ZFP-91, following selective uncaging of SNS-032 or thalidomide, respectively (FIGS. 40A-40B, 41A-41B). Collectively, these findings underscore the modularity and broad applicability of the dual-caging approach for stimulus-responsive bifunctional degraders.

In summary, we report DAO-PROTACs, a dual-caging strategy designed to enhance specificity in targeted protein degradation. We show here that: (i) DAO-PROTACs can be generated by introducing “bumps” at secondary amides in the linker proximal to either of the ligand moieties; (ii) concurrent presence of both stimuli, hypoxia and Cath-L, is required for activating protein degradation; (iii) DAO-PROTACs are substantially less cytotoxic than mono-masked PROTACs even in the absence of the relevant stimulus; (iv) similar advantage in safety is also observed in heathy cells that inherently lack the stimuli; and (v) the mono-masked PROTACs engage either the POI or the E3 ligase to cause downstream cytotoxicity, an effect that is mitigated in DAO-PROTACs. Overall, our findings suggest DAO-PROTACs would not only enhance target specificity because of the requirement of having two stimuli, but also offer to minimize off-target toxicity.

### Materials

All reagents were obtained from commercial sources and used without further purification, unless otherwise mentioned. Sodium chloride (NaCl), anhydrous sodium sulphate (Na₂SO₄), sodium bicarbonate (NaHCO₃) N,N-diisopropylethylamine (DIPEA), sodium triacetoxyborohydride (NaBH(OAc)₃), potassium carbonate (K₂CO₃), trifluoroacetic acid (TFA), formic acid, dichloromethane (DCM), methanol, acetonitrile, water (HPLC-grade), tetrahydrofuran, dry dimethylformamide (DMF), 4-nitrobenzaldehyde, tert-butyl (4-aminobutyl)carbamate, ε-acetyl-N-Boc-(L)-lysine, were purchased from Fisher Scientific. Pyridinium chlorochromate (PCC) and 2-ethoxy-1-ethoxycarbonyl-1,2-dihydroquinoline (EEDQ) were purchased from Sigma-Aldrich. Hexafluorophosphate azabenzotriazole tetramethyl uronium (HATU) was purchased from Chem Impex. JQ1 carboxylic acid and thalidomide-O—COOH were purchased from Med-Chem-Express.

### Synthetic Methods

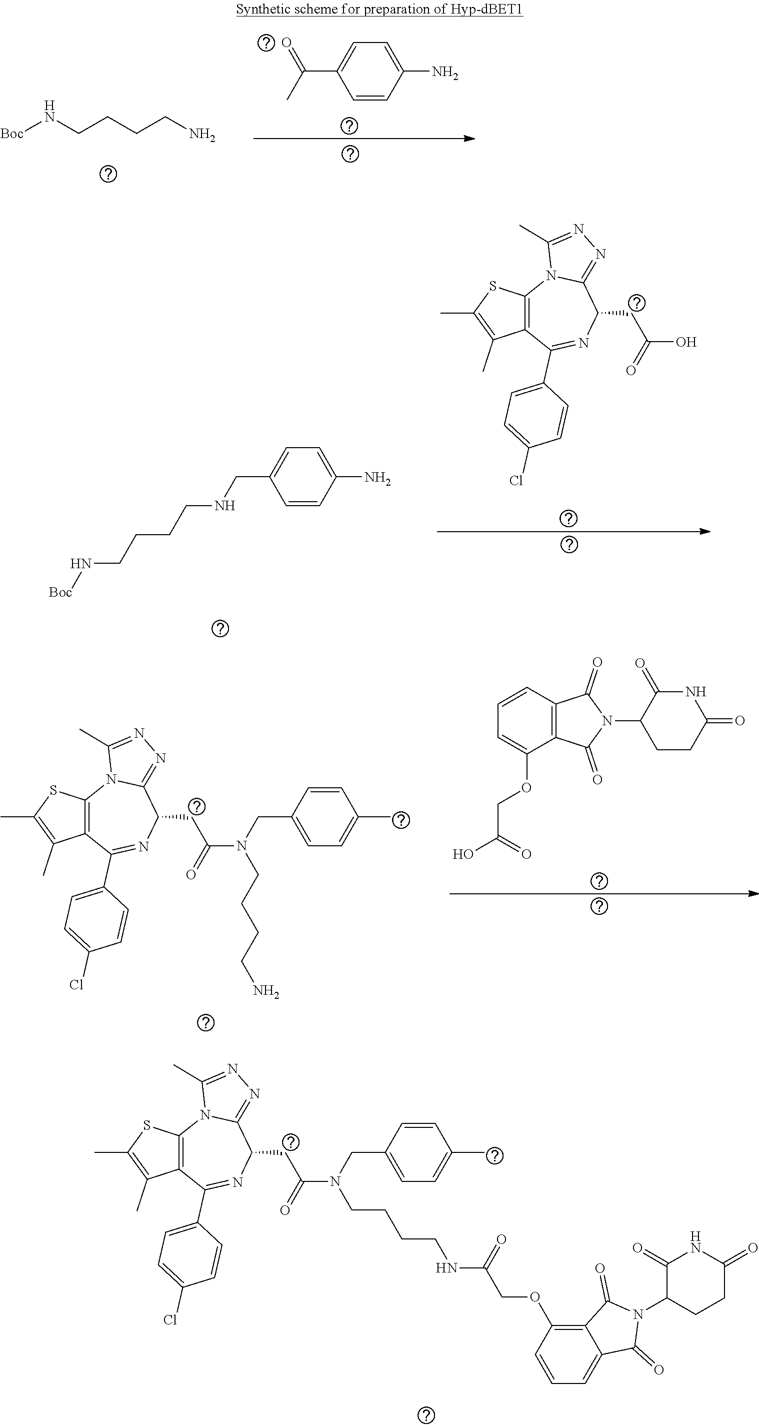

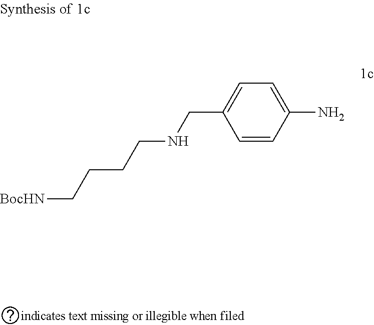 [SMILES: CC(C)(C)OC(=O)NCCCCNCc1ccc(N)cc1]

200 mg (1.33 mmol) of 4-nitrobenzaldehyde (1b) was dissolved in dry dichloromethane (5 mL) and added to a round bottom flask with excess anhydrous sodium sulphate under inert atmosphere and the reaction mixture was stirred for 10 minutes. To this, 300 mg (1.59 mmol) N-Boc-1,4-diaminobutane (1a) was added dropwise. The reaction mixture was then stirred for 12 h, following which, sodium triacetoxyborohydride (1.91 mmol) was then added and left to stir for 4 h. The reaction mixture was concentrated in vacuo, then extracted with DCM and aqueous sodium bicarbonate (×2) and brine (×1). The combined extracts were dried over anhydrous Na₂SO₄, and the crude product purified by silica gel chromatography using DCM and methanol as eluents to afford 1c in 79% yield. ¹H NMR (400 MHz, (CD₃)₂CO) δ (ppm) 8.30-8.11 (m, 2H), 7.76-7.61 (m, 2H), 3.94 (s, 2H), 3.13-2.98 (m, 2H), 2.72-2.57 (m, 2H), 2.08 (d, J=0.9 Hz, 1H), 2.07 (d, J=2.3 Hz, 1H), 1.54 (quint, J=3.4 Hz, 4H), 1.39 (s, 9H). ¹³C NMR (100 MHz, (CD₃)₂CO) δ (ppm) 172.4, 156.6, 149.9, 147.8, 129.8, 124.0, 78.3, 53.2, 49.4, 40.8, 28.6, 28.5, 27.7, 20.7. [M+H]⁺ calculated for: C₁₆H₂₅N₃O₄: 324.1923, [M+H]⁺ from HRMS (ESI): 324.1914.

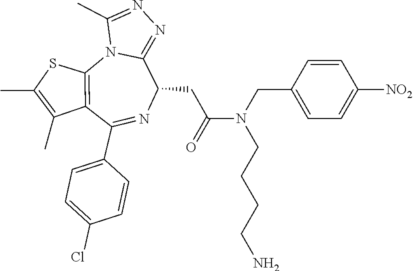 [SMILES: Cc1sc2c(c1C)C(c1ccc(Cl)cc1)=N[C@@H](CC(=O)N(CCCCN)Cc1ccc([N+](=O)[O-])cc1)c1nnc(C)n1-2]

74 mg (0.19 mmol) of JQ1 carboxylic acid (1d), 70 mg (0.19 mmol) of HATU, and 42 μL (0.23 mmol) DIPEA was dissolved in dry dimethylformamide (3 mL) and added to a vial under inert atmosphere and the reaction mixture was stirred for 30 minutes. To this, 50 mg (0.15 mmol) 1c and DIPEA (0.23 mmol) were added dropwise to the reaction mixture and left to stir for 12 h. The reaction mixture was then concentrated in vacuo, extracted with DCM and aqueous sodium bicarbonate (×2) and brine (×1). The combined extracts were dried over anhydrous Na₂SO₄, and the crude product purified by silica gel chromatography using DCM and methanol as eluents. The product was carried over to the next step without further characterization. In that step, the N-Boc group was deprotected using TFA in methanol (1:1) and stirred overnight. The resulting mixture was co-evaporated with methanol thrice and further concentrated in vacuo to afford 1e in 60% yield. ¹H NMR (400 MHz, CD₃OD) δ 8.32-8.22 (m, 2H), 7.73 (d, J=8.6 Hz, 1H), 7.58 (d, J=6.9 Hz, 1H), 7.44 (dd, J=8.5, 1.4 Hz, 4H), 4.81 (d, J=3.6 Hz, 2H), 4.76 (dd, J=12.9, 6.9 Hz, 1H), 3.74 (dd, J=7.2, 4.6 Hz, 2H), 3.60-3.47 (m, 2H), 3.04-2.92 (m, 2H), 2.74 (d, J=11.0 Hz, 3H), 2.51-2.46 (m, 3H), 1.97-1.74 (m, 4H), 1.74-1.70 (m, 3H), 1.31 (s, 2H). ¹³C NMR (100 MHz, (CD₃)₂CO) δ (ppm) 171.2, 164.3, 156.6, 147.9, 147.6, 138.2, 136.7, 131.7, 131.2, 131.0, 131.0, 129.5, 129.3, 128.9, 124.5, 124.2, 124.1, 55.9, 48.9, 48.3, 47.7, 36.0, 26.8, 25.5, 14.5, 12.9, 11.7. [M+H]⁺ calculated for C₃₀H₃₂ClN₇O₃S: 606.2054, [M+H]⁺ from HRMS (ESI): 606.1566.

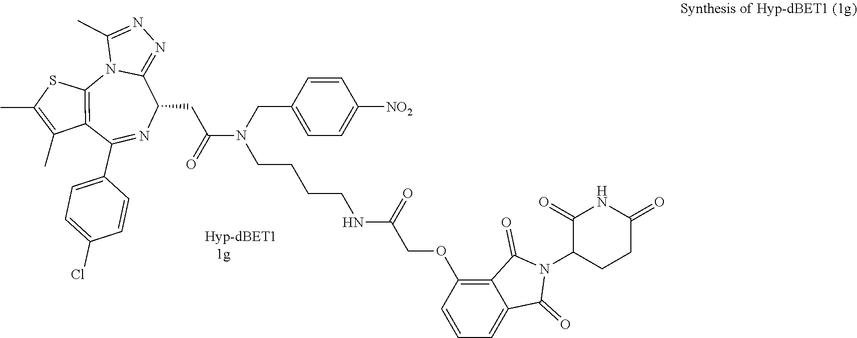 [SMILES: Cc1sc2c(c1C)C(c1ccc(Cl)cc1)=N[C@@H](CC(=O)N(CCCCNC(=O)COc1cccc3c1C(=O)N(C1CCC(=O)NC1=O)C3=O)Cc1ccc([N+](=O)[O-])cc1)c1nnc(C)n1-2]

7 mg (0.019 mmol) of Thalidomide-O—COOH (if), 8 mg (0.019 mmol) HATU and 4.4 μL (0.025 mmol) DIPEA was dissolved in 1 mL dry dimethylformamide and added to a vial under inert atmosphere and the reaction mixture was stirred for 30 minutes. To this, 10 mg (0.016 mmol) 1e and DIPEA (0.025 mmol) was added dropwise to the reaction mixture and left to stir for 12 h. The reaction mixture was then concentrated in vacuo, extracted with DCM and aqueous sodium bicarbonate (×2) and brine (×1). The combined extracts were dried over anhydrous Na₂SO₄, and the crude product purified by reverse phase C-18 gel chromatography using water and acetonitrile as eluents to afford 1g in 63% yield. ¹H NMR (400 MHz, (CD₃)₂CO) δ (ppm) 10.07-9.99 (m, 1H), 8.25-8.09 (m, 2H), 7.78-7.66 (m, 2H), 7.60 (dd, J=8.6, 6.1 Hz, 2H), 7.45 (m, 5H), 7.39 (s, 1H), 5.14-5.07 (m, 1H), 4.80-4.70 (m, 4H), 3.82-3.29 (m, 6H), 2.98-2.89 (m, 2H), 2.78-2.67 (m, 2H), 2.63-2.54 (m, 3H), 2.44 (s, 3H), 2.30-2.12 (m, 1H), 2.03-1.73 (m, 2H), 1.70 (s, 3H), 1.69-1.49 (m, 2H). ¹³C NMR (100 MHz, (CD₃)₂CO) δ (ppm) 171.7, 170.5, 169.1, 166.8, 166.6, 166.1, 163.4, 155.6, 155.0, 154.8, 149.7, 149.6, 146.9, 146.8, 146.1, 137.3, 137.0, 136.8, 135.9, 133.5, 132.6, 130.8, 130.3, 130.1, 128.5, 128.5, 128.4, 128.0, 123.6, 123.2, 120.5, 117.9, 116.2, 68.3, 54.9, 50.3, 49.3, 48.3, 47.8, 45.6, 37.6, 35.1, 35.1, 31.0, 26.3, 25.8, 22.3, 13.6, 12.1, 10.8. [M+H]⁺ calculated for C₄₅H₄₂ClN₉O₉S: 920.2593, [M+H]⁺ from HRMS (ESI): 920.1435.

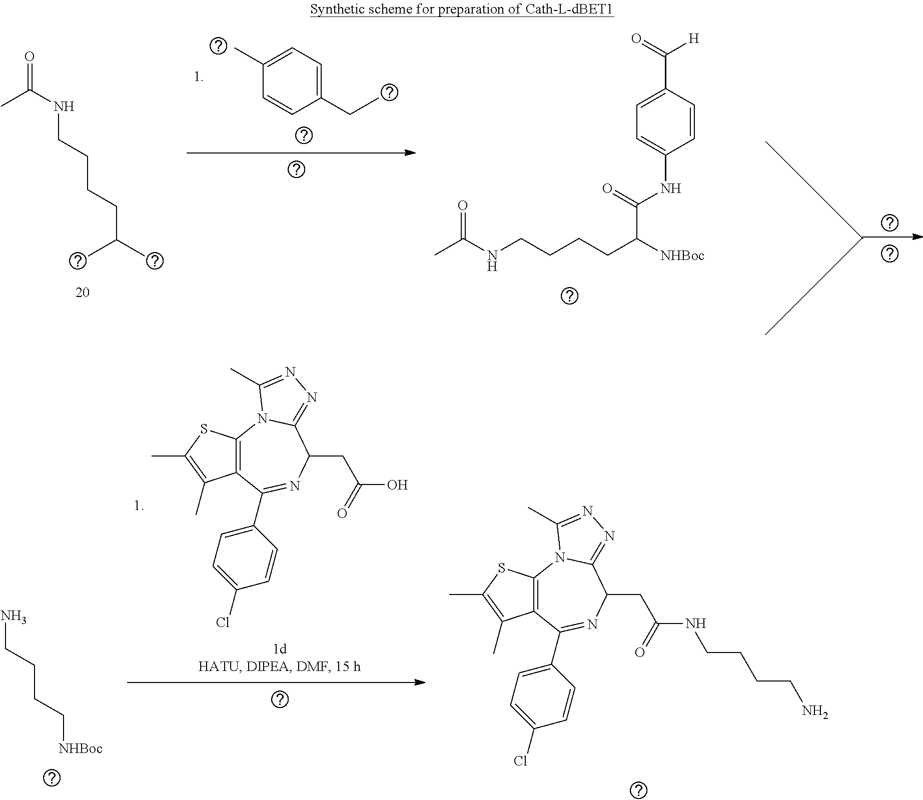

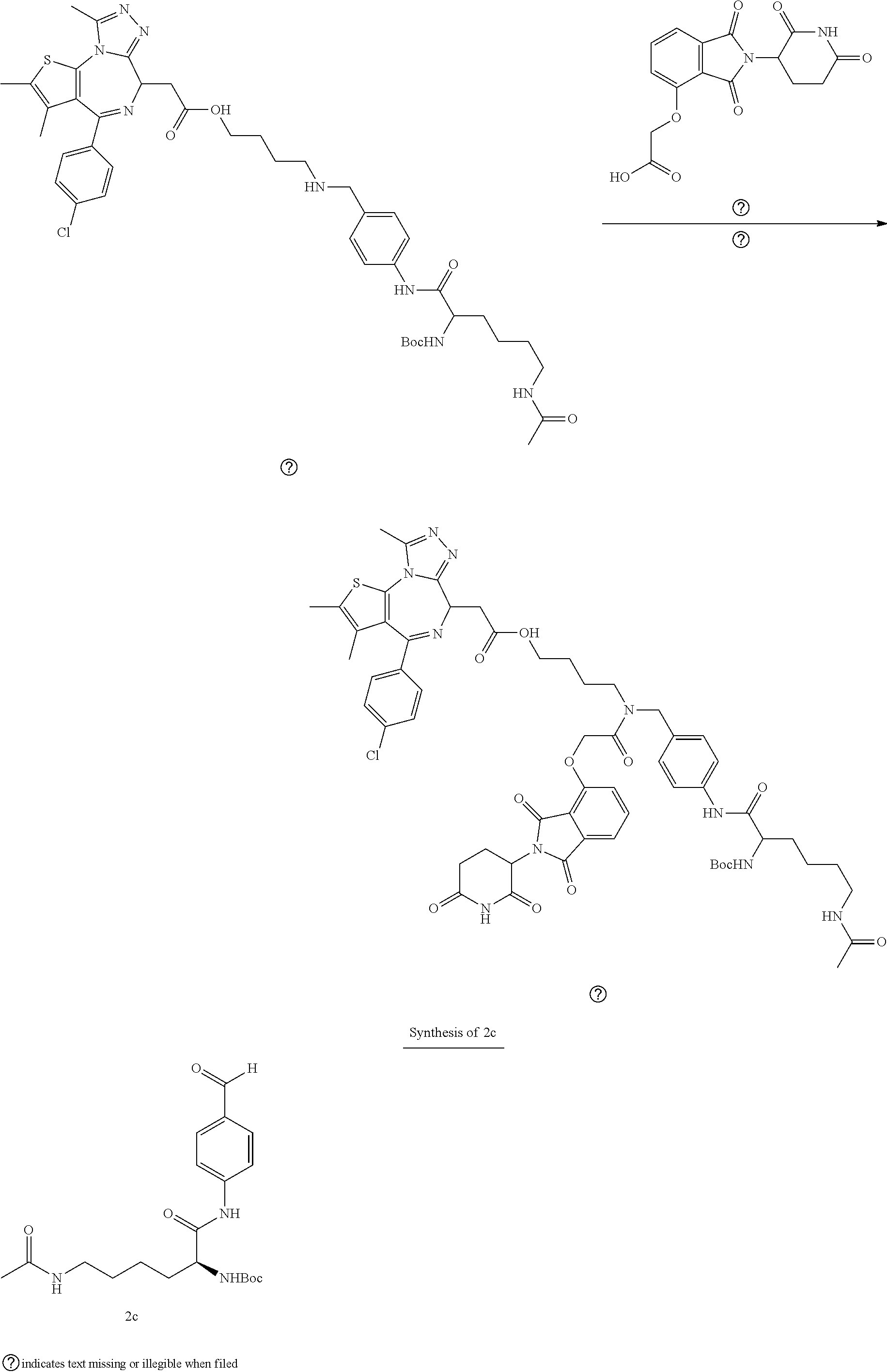 [SMILES: CC(=O)NCCCCC(NC(=O)OC(C)(C)C)C(=O)Nc1ccc(CNCCCCOC(=O)CC2N=C(c3ccc(Cl)cc3)c3c(sc(C)c3C)-n3c(C)nnc32)cc1 ; O=C(O)COc1cccc2c1C(=O)N(C1CCC(=O)NC1=O)C2=O ; CC(=O)NCCCCC(NC(=O)OC(C)(C)C)C(=O)Nc1ccc(CN(CCCCOC(=O)CC2N=C(c3ccc(Cl)cc3)c3c(sc(C)c3C)-n3c(C)nnc32)C(=O)COc2cccc3c2C(=O)N(C2CCC(=O)NC2=O)C3=O)cc1 ; CC(=O)NCCCC[C@H](NC(=O)OC(C)(C)C)C(=O)Nc1ccc(C=O)cc1]

100 mg (0.35 mmol) of ε-acetyl-N-Boc-(L)-lysine (2a) was dissolved in methanol (0.5 mL) and added to a vial. 43 mg (0.35 mmol) 4-aminobenzyl alcohol (2b), and 103 mg (0.42 mmol) of EEDQ was dissolved in 2 mL of DCM added dropwise to the same vial and the reaction mixture was stirred for 12 h. The reaction mixture was then concentrated in vacuo, then extracted with DCM and aqueous sodium bicarbonate (×2) and brine (×1). The combined extracts were dried over anhydrous Na₂SO₄, and the crude product purified by silica gel chromatography using DCM and methanol as eluents to afford the product 2c-1 at 81% yield. The product was carried over to the next step without further characterization. The product 2c-1 (81 mg, 0.23 mmol) was then oxidized using pyridinium chlorochromate (39 mg, 0.18 mmol) in DCM. The reaction mixture was concentrated in vacuo, then extracted with DCM and aqueous sodium bicarbonate (×2) and brine (×1). The combined extracts were dried over anhydrous Na₂SO₄, and the crude product purified by silica gel chromatography using DCM and methanol as eluents to afford 2c at 83% yield. ¹H NMR (500 MHz, CD₃OD) δ (ppm) 9.78 (s, 1H), 7.78 (d, J=8.5 Hz, 2H), 7.71 (d, J=8.5 Hz, 2H), 4.07 (dd, J=9.0, 5.3 Hz, 1H), 3.09-3.05 (m, 2H), 1.80 (s, 3H), 1.60 (m, 2H), 1.44 (q, J=6.5 Hz, 2H), 1.35 (s, 9H), 1.31-1.28 (m, 2H). ¹³C NMR (100 MHz, CD₃OD) δ (ppm) 191.5, 172.7, 171.8, 156.6, 144.2, 132.2, 130.5, 119.3, 79.3, 55.4, 48.4, 48.2, 48.1, 48.0, 47.9, 47.8, 47.7, 47.6, 47.4, 47.2, 47.0, 38.7, 31.6, 28.6, 27.3, 22.9, 21.1. [M+H]⁺ calculated for C₂₀H₂₉N₃O₅: 392.2185, [M+H]⁺ from HRMS (ESI): 392.2483.

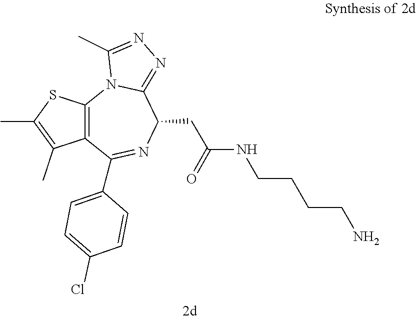 [SMILES: Cc1sc2c(c1C)C(c1ccc(Cl)cc1)=N[C@@H](CC(=O)NCCCCN)c1nnc(C)n1-2]

20 mg (0.05 mmol) of JQ1-carboxylic acid (1d), 19 mg (0.05 mmol) HATU and 22 μL (0.12 mmol) DIPEA was dissolved in dry dimethylformamide (2 mL) and added to a vial under inert atmosphere and the reaction mixture was stirred for 30 minutes. To this, 7.8 mg (0.041 mmol) N-Boc-1,4-diaminobutane (a) and DIPEA (0.12 mmol) was added dropwise to the reaction mixture and left to stir for 12 h. The reaction mixture was then concentrated in vacuo, extracted with DCM and aqueous sodium bicarbonate (×2) and brine (×1). The combined extracts were dried over anhydrous Na₂SO₄, and the crude product purified by reverse phase C-18 gel chromatography using water and acetonitrile as eluents. The product was carried over to the next step without further characterization. In that step, the N-Boc group was deprotected using TFA in DCM (1:1) and stirred overnight. The resulting mixture was co-evaporated with methanol thrice and further concentrated in vacuo to afford to afford 2d in 81% yield. ¹H NMR (400 MHz, (CD₃)₂CO) δ (ppm) 7.63 (broad, 1H), 7.51 (d, J=8.6 Hz, 2H), 7.44 (d, J=8.7 Hz, 2H), 6.07 (s, 1H), 4.65 (t, J=7.0 Hz, 1H), 3.35-3.32 (m, 2H), 3.31-3.27 (m, 1H), 3.25 (dd, J=7.3, 5.1 Hz, 1H), 2.64 (s, 3H), 2.47 (s, 3H), 1.72 (s, 3H), 1.57 (quint, J=3.3 Hz, 4H), 1.40 (s, 9H). ¹³C NMR (100 MHz, (CD₃)₂CO) δ (ppm) 170.7, 164.2, 156.8, 156.4, 150.6, 138.2, 136.6, 133.5, 131.6, 131.2, 131.1, 131.0, 129.2, 78.4, 55.2, 40.8, 39.4, 39.1, 28.0, 27.6, 14.5, 12.9, 11.7. [M+H]⁺ calculated for C₂₃H₂₇ClN₆₀S: 471.1734, [M+H]⁺ from HRMS (ESI): 471.1747.

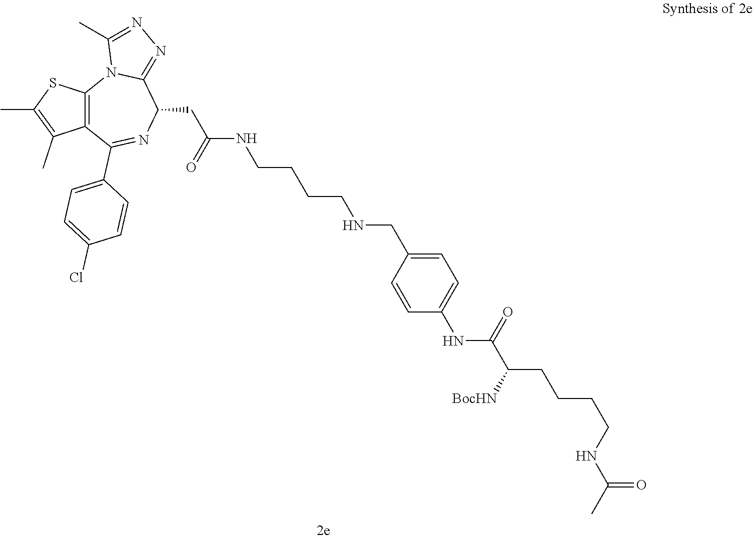 [SMILES: CC(=O)NCCCC[C@H](NC(=O)OC(C)(C)C)C(=O)Nc1ccc(CNCCCCNC(=O)C[C@@H]2N=C(c3ccc(Cl)cc3)c3c(sc(C)c3C)-n3c(C)nnc32)cc1]

15 mg (0.032 mmol) of 2c was dissolved in dry tetrahydrofuran (0.5 mL) and added to vial with excess anhydrous sodium sulphate under inert atmosphere and the reaction mixture was stirred for 10 minutes. To this, 10 mg (0.026 mmol) 2d was dissolved in dry tetrahydrofuran (0.5 mL) and added dropwise, and the reaction mixture was stirred for 12 h. Sodium triacetoxyborohydride (0.032 mmol) was then added to the reaction mixture and stirred overnight. The reaction mixture was concentrated in vacuo, then extracted with DCM and aqueous sodium bicarbonate (×2) and brine (×1). The combined extracts were dried over anhydrous Na₂SO₄, and the crude product purified by silica gel chromatography using DCM and methanol as eluents to afford 2e in 29% yield. ¹H NMR (500 MHz, (CD₃)₂SO) δ (ppm) 9.85 (s, 1H), 8.12 (t, J=5.7 Hz, 1H), 7.74 (t, J=5.7 Hz, 1H), 7.45 (d, J=8.2 Hz, 2H), 7.40 (d, J=8.5 Hz, 2H), 7.35 (d, J=8.1 Hz, 2H), 7.17 (d, J=8.1 Hz, 2H), 7.02 (d, J=8.1 Hz, 1H), 6.91 (d, J=8.0 Hz, 1H), 4.71 (s, 1H), 4.45-4.41 (m, 1H), 3.95 (q, J=7.5 Hz, 2H), 3.57 (s, 2H), 3.15 (s, 1H), 3.09 (q, J=5.6 Hz, 2H), 2.94-2.90 (m, 2H), 2.52 (s, 3H), 2.34 (s, 3H), 1.69 (s, 3H), 1.54 (s, 3H), 1.40 (t, J=5.2 Hz, 4H), 1.31 (s, 9H), 1.24-1.18 (m, 4H), 1.17 (s, 2H). ¹³C NMR (100 MHz, (CD₃)₂SO) δ (ppm) 171.2, 171.2, 169.3, 168.8, 163.0, 155.4, 155.1, 149.8, 137.5, 136.7, 135.2, 132.2, 130.6, 130.1, 129.8, 129.5, 128.4, 128.2, 118.9, 77.9, 54.9, 53.9, 52.4, 48.1, 38.4, 38.3, 37.6, 31.6, 31.2, 30.1, 28.9, 28.2, 27.1, 26.6, 23.1, 22.6, 14.0, 12.6, 11.3, 1.1. [M+H]⁺ calculated for C₄₃H₅₆ClN₉O₅S: 845.3892, [M+H]⁺ from HRMS (ESI): 845.3826.

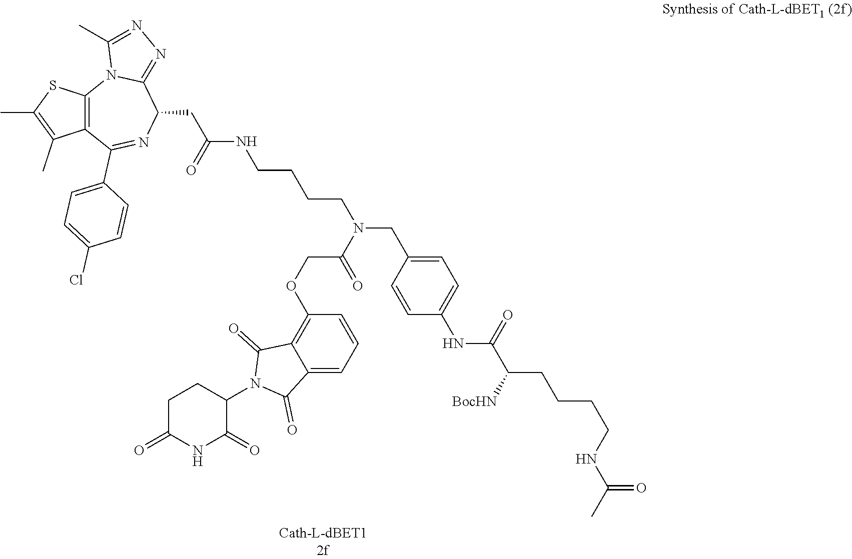 [SMILES: CC(=O)NCCCC[C@H](NC(=O)OC(C)(C)C)C(=O)Nc1ccc(CN(CCCCNC(=O)C[C@@H]2N=C(c3ccc(Cl)cc3)c3c(sc(C)c3C)-n3c(C)nnc32)C(=O)COc2cccc3c2C(=O)N(C2CCC(=O)NC2=O)C3=O)cc1]

7 mg (0.019 mmol) Thalidomide-O—COOH (if), 8 mg (0.019 mmol) HATU and 4.4 μL (0.025 mmol) DIPEA was dissolved in dry dimethylformamide (1 mL) and added to a vial under inert atmosphere and the reaction mixture was stirred for 30 minutes. To this, 10 mg (0.016 mmol) 2e and DIPEA (0.025 mmol) was added dropwise to the reaction mixture and left to stir for 12 h. The reaction mixture was then concentrated in vacuo, extracted with DCM and aqueous sodium bicarbonate (×2) and brine (×1). The combined extracts were dried over anhydrous Na₂SO₄, and the crude product purified by reverse phase C-18 gel chromatography using water and acetonitrile as eluents to afford 2f in 45% yield. ¹H NMR (400 MHz, CD₃OD) δ (ppm) 7.91-7.73 (m, 1H), 7.68 (d, J=8.2 Hz, 1H), 7.57 (d, J=8.3 Hz, 2H), 7.48-7.43 (m, 4H), 7.42 (d, J=4.9 Hz, 1H), 7.39 (t, J=7.1 Hz, 2H), 4.65 (t, J=7.3 Hz, 1H), 4.15 (s, 1H), 3.78 (t, J=6.1 Hz, 1H), 3.48 (t, J=1.7 Hz, 1H), 3.42 (d, J=7.4 Hz, 2H), 3.39 (s, 2H), 3.37 (s, 2H), 3.17 (t, J=6.8 Hz, 2H), 3.13 (t, J=1.7 Hz, 1H), 2.96 (t, J=6.6 Hz, 1H), 2.71-2.66 (m, 3H), 2.46 (s, 3H), 1.94 (s, 2H), 1.90 (s, 3H), 1.89 (s, 1H), 1.78 (d, J=7.6 Hz, 2H), 1.73 (s, 2H), 1.70 (d, J=3.7 Hz, 3H), 1.68-1.64 (m, 2H), 1.55 (dd, J=12.3, 5.3 Hz, 4H), 1.45 (s, 9H), 1.29 (s, 2H). [M+Na]⁺ calculated for C₅₈H₆₆ClN₁₁NaO₁₁S: 1182.425, [M+Na]⁺ from MALDI-ToF spectrometry: 1182.992.

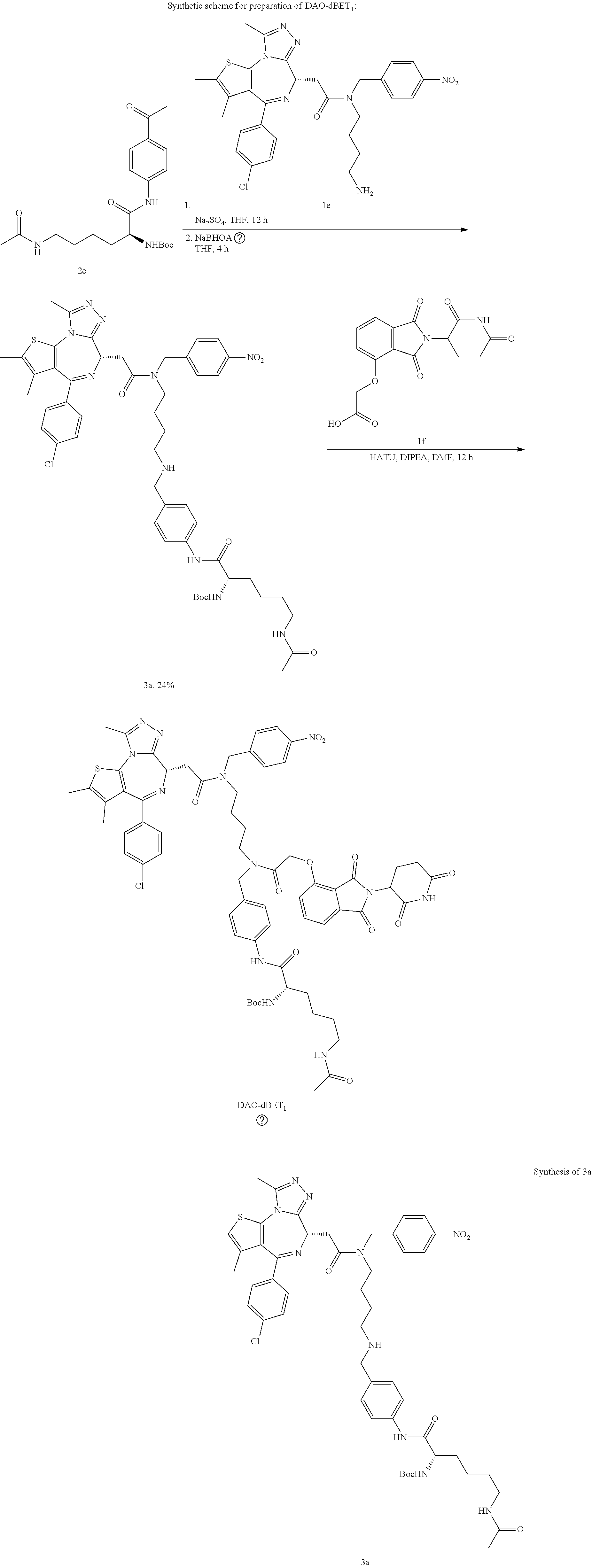

16 mg (0.026 mmol) of 2c was dissolved in dry tetrahydrofuran (500 μL) and added to vial with excess anhydrous sodium sulphate under inert atmosphere and the reaction mixture was stirred for 10 minutes. To this, 9 mg (0.026 mmol) 1e was dissolved in 500 μL dry THF and added dropwise, and the reaction mixture was stirred for 12 h. Sodium triacetoxyborohydride (0.186 mmol) was then added to the reaction mixture and stirred overnight. The reaction mixture was concentrated in vacuo, then extracted with DCM and aqueous sodium bicarbonate (×2) and brine (×1). The combined extracts were dried over anhydrous Na₂SO₄, and the crude product purified by silica gel chromatography using DCM and methanol as eluents to afford 3a in 24% yield. ¹H NMR (500 MHz, (CD₃)₂SO) δ 9.85 (s, 1H), 8.12 (t, J=5.7 Hz, 2H), 7.74 (t, J=5.7 Hz, 2H), 7.45 (d, J=8.2 Hz, 2H), 7.40 (d, J=8.5 Hz, 2H), 7.35 (d, J=8.1 Hz, 2H), 7.17 (d, J=8.1 Hz, 2H), 7.02 (d, J=8.1 Hz, 1H), 6.91 (d, J=8.0 Hz, 1H), 4.43 (dd, J=8.3, 6.1 Hz, 1H), 3.95 (q, J=7.5 Hz, 2H), 3.57 (s, 2H), 3.16 (d, J=8.3 Hz, 2H), 3.10 (d, J=5.5 Hz, 2H), 3.00 (t, J=5.7 Hz, 2H), 2.94-2.90 (m, 2H), 2.52 (s, 3H), 2.34 (s, 3H), 1.69 (s, 3H), 1.54 (s, 3H), 1.40 (t, J=5.2 Hz, 4H), 1.31 (s, 9H), 1.24-1.18 (m, 4H), 1.17 (s, 2H). ¹³C NMR (100 MHz, (CD₃)₂SO) δ (ppm) 170.1, 163.1, 155.2, 149.8, 146.7, 146.5, 146.4, 136.7, 135.2, 132.2, 130.7, 130.1, 129.9, 129.6, 128.4, 128.3, 127.8, 123.7, 123.3, 69.7, 54.5, 54.2, 49.9, 48.0, 47.5, 45.4, 34.7, 30.7, 29.0, 25.6, 25.6, 24.2, 20.6, 14.0, 12.7, 11.3. [M+H]⁺ calculated for C₅₀H₆₁ClN₁₀O₇S: 981.4212, [M+H]⁺ from HRMS (ESI): 981.1497.

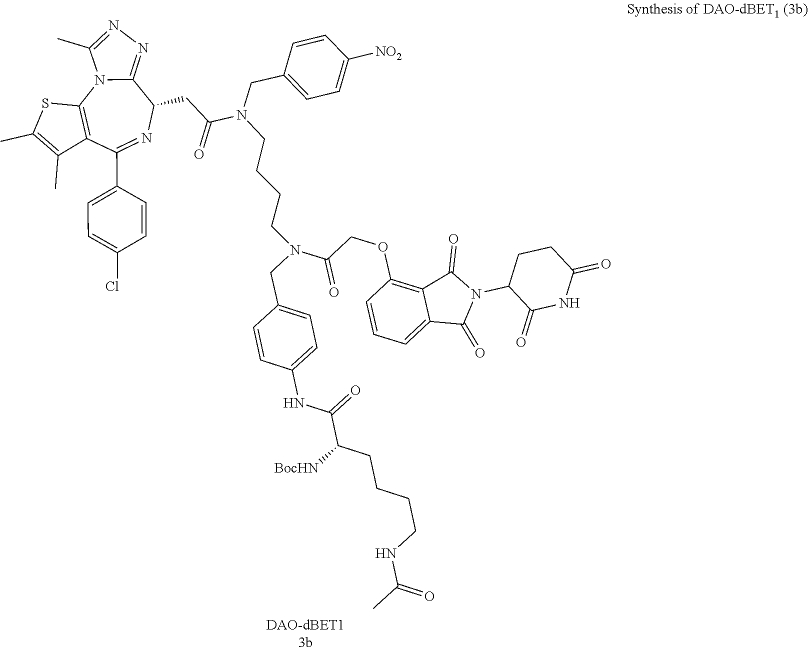

7 mg (0.019 mmol) Thalidomide-O—COOH (1f), 8 mg (0.019 mmol) HATU and 4.4 μL (0.025 mmol) DIPEA was dissolved in dry dimethylformamide (1 mL) and added to a vial under inert atmosphere and the reaction mixture was stirred for 30 minutes. To this, 10 mg (0.016 mmol) 3a and DIPEA (0.025 mmol) was added dropwise to the reaction mixture and left to stir for 12 h. The reaction mixture was then concentrated in vacuo, extracted with DCM and aqueous sodium bicarbonate (×2) and brine (×1). The combined extracts were dried over anhydrous Na₂SO₄, and the crude product purified by reverse phase C-18 gel chromatography using water and acetonitrile as eluents to afford 3b in 56% yield. ¹H NMR (400 MHz, CD₃OD) δ (ppm) 8.28 (d, J=8.7 Hz, 1H), 8.21 (d, J=8.7 Hz, 2H), 7.70 (d, J=8.6 Hz, 2H), 7.63 (d, J=8.0 Hz, 2H), 7.55 (d, J=8.6 Hz, 2H), 7.42 (d, J=3.9 Hz, 4H), 7.40 (s, 1H), 7.37 (d, J=8.3 Hz, 2H), 4.79 (s, 2H), 4.76-4.72 (m, 1H), 4.15 (s, 2H), 4.10 (s, 2H), 3.78-3.66 (m, 4H), 3.49-3.47 (m, 1H), 3.17 (d, J=5.7 Hz, 2H), 3.14-3.13 (m, 1H), 3.11 (d, J=15.3 Hz, 2H), 2.73 (s, 3H), 2.65 (s, 1H), 2.45 (s, 3H), 1.99 (s, 1H), 1.90 (s, 3H), 1.84 (s, 4H), 1.70 (s, 3H), 1.53 (d, J=6.8 Hz, 4H), 1.44 (s, 9H), 1.38 (d, J=7.0 Hz, 2H), 1.29 (s, 2H). ¹³C NMR (100 MHz, (CD₃)₂SO) δ 173.25, 170.57, 170.39, 167.27, 167.17, 165.96, 165.71, 163.61, 156.19, 155.67, 155.46, 150.30, 147.10, 146.91, 146.60, 137.16, 135.72, 133.48, 132.68, 131.21, 130.59, 130.37, 130.07, 128.95, 128.81, 128.70, 128.26, 124.20, 123.82, 120.52, 117.26, 116.51, 115.77, 88.93, 68.13, 60.92, 60.77, 54.99, 54.76, 49.26, 48.49, 46.04, 40.62, 40.41, 40.20, 39.99, 39.78, 39.57, 39.36, 35.20, 31.41, 30.01, 28.68, 23.08, 22.46, 14.50, 13.16, 11.77. [M+H]⁺ calculated for C₆₅H₇₁ClN₁₂O₁₃S: 1295.4751, [M+H]⁺ from HRMS (ESI): 1295.3754.

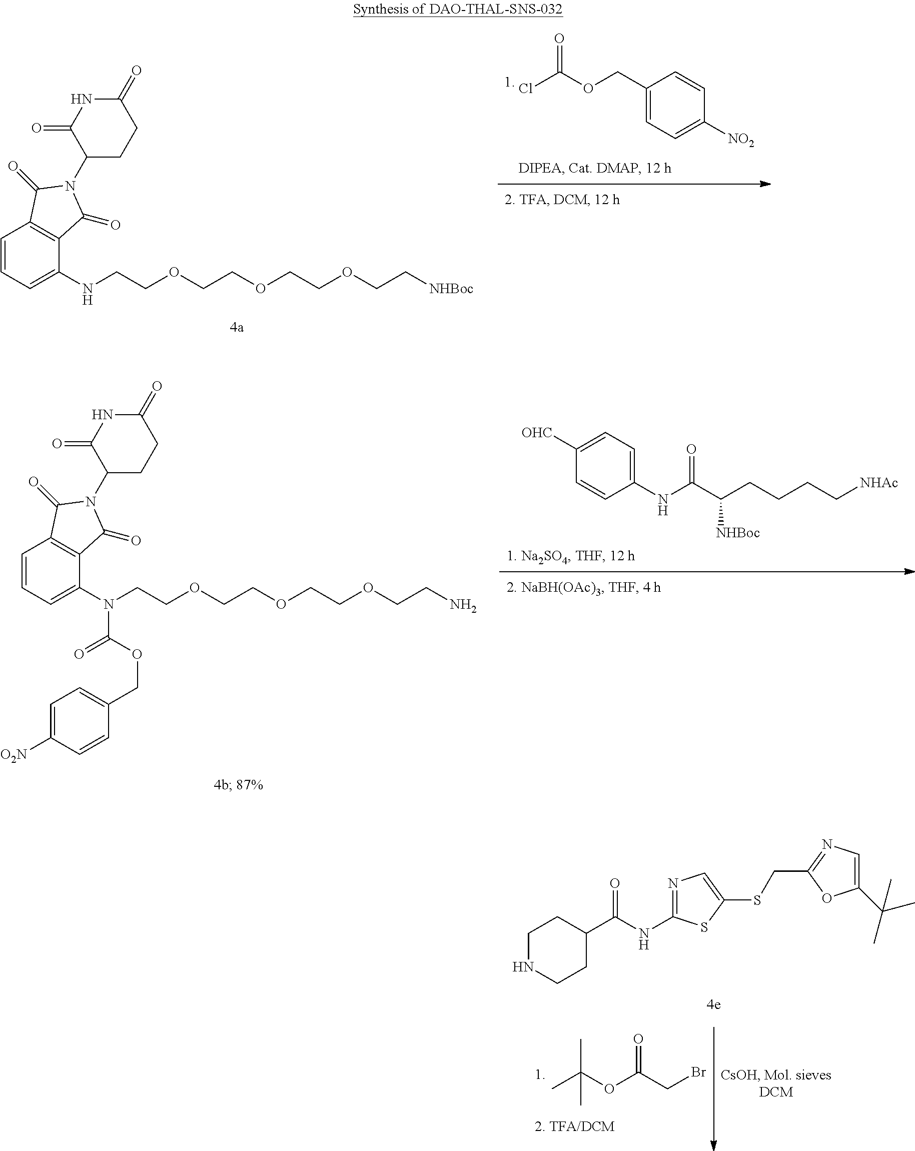

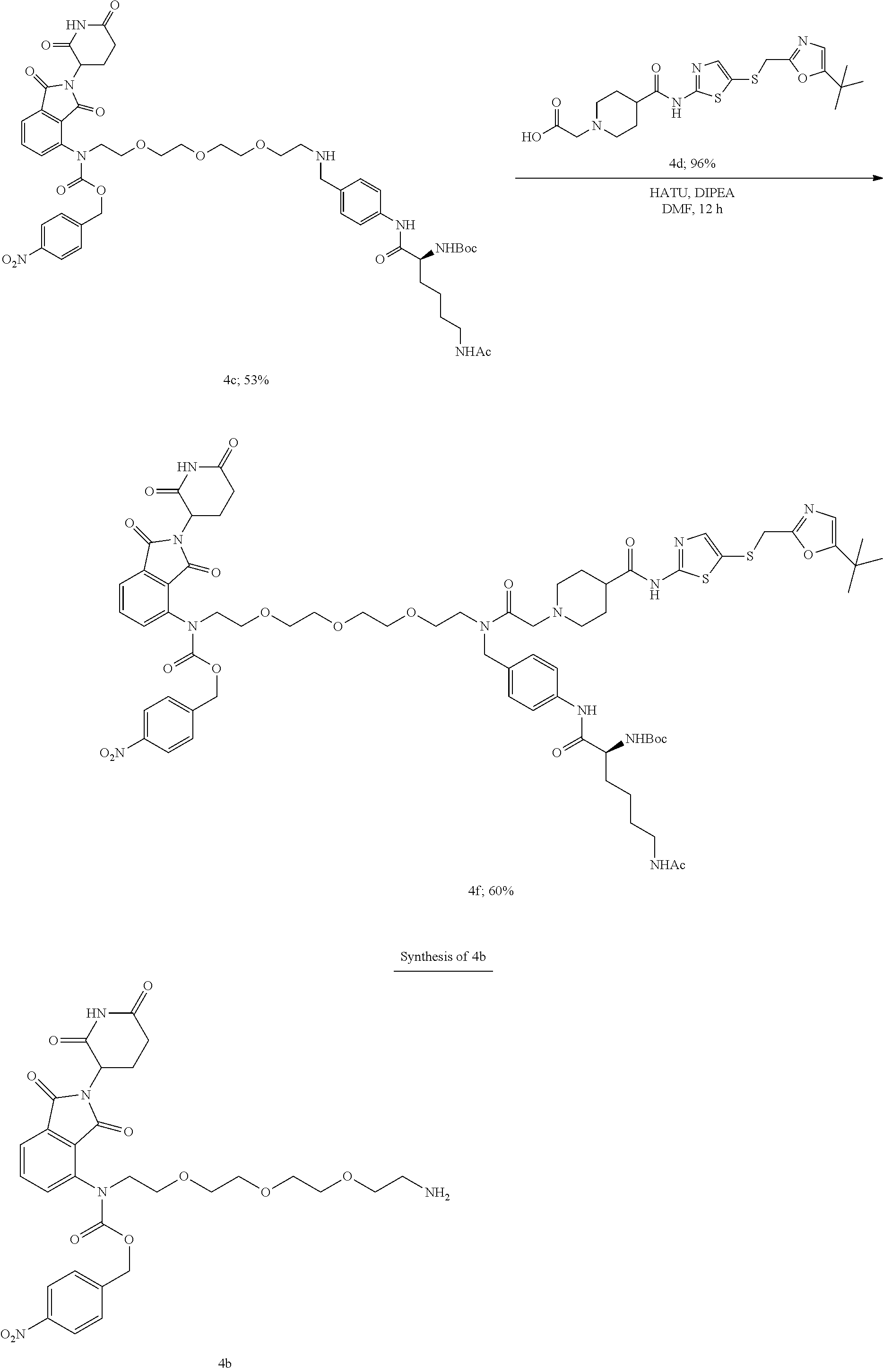 [SMILES: CC(=O)NCCCC[C@H](NC(=O)OC(C)(C)C)C(=O)Nc1ccc(CNCCOCCOCCOCCN(C(=O)OCc2ccc([N+](=O)[O-])cc2)c2cccc3c2C(=O)N(C2CCC(=O)NC2=O)C3=O)cc1 ; CC(C)(C)c1cnc(CSc2cnc(NC(=O)C3CCN(CC(=O)O)CC3)s2)o1 ; CC(=O)NCCCC[C@H](NC(=O)OC(C)(C)C)C(=O)Nc1ccc(CN(CCOCCOCCOCCN(C(=O)OCc2ccc([N+](=O)[O-])cc2)c2cccc3c2C(=O)N(C2CCC(=O)NC2=O)C3=O)C(=O)CN2CCC(C(=O)Nc3ncc(SCc4ncc(C(C)(C)C)o4)s3)CC2)cc1 ; NCCOCCOCCOCCN(C(=O)OCc1ccc([N+](=O)[O-])cc1)c1cccc2c1C(=O)N(C1CCC(=O)NC1=O)C2=O]

20 mg (0.036 mmol) thalidomide-NH-PEG3-NH-Boc (4a), 9 mg (0.072 mmol) DIPEA and catalytic DMAP was dissolved in anhydrous dimethylformamide (500 μL) and added to a vial under inert atmosphere and the reaction mixture was stirred for 10 minutes. To this, 9 mg (0.045 mmol) 4-nitrophenyl chloroformate dissolved in anhydrous dimethylformamide (500 μL) was added dropwise to the reaction mixture at 4° C. and left to stir for 12 h at room temperature. The reaction mixture was then concentrated in vacuo, extracted with DCM and aqueous sodium bicarbonate (×2) and brine (×1). The combined extracts were dried over anhydrous Na2SO4, and the crude product purified by silica gel chromatography using hexanes and ethyl acetate as eluents to afford 4b in 87% yield. ¹H NMR (500 MHz, CDCl3) δ 8.39 (s, 1H), 7.48 (dd, J=8.5, 7.1 Hz, 2H) 7.10 (d, J=7.1 Hz, 2H), 6.91 (d, J=8.5 Hz, 2H), 6.48 (broad, 1H), 5.11 (s, 2H), 4.92-4.89 (m, 1H), 3.73-3.71 (m, 2H), 3.67 (s, 2H), 3.65-3.63 (m, 2H), 3.61-3.60 (m, 2H), 3.53-3.51 (m, 2H), 3.48-3.46 (m, 2H), 3.30 (broad, 1H), 2.89-2.83 (m, 2H), 2.77-2.73 (m, 2H), 2.11 (m, 2H), 1.43 (s, 9H), 1.25 (s, 1H). 13C NMR (100 MHz, CDCl3) δ 171.1, 169.3, 167.6, 156.0, 146.8, 136.0, 132.5, 116.8, 111.7, 110.3, 70.7, 70.6, 70.2, 69.5, 48.9, 42.4, 31.4, 28.4, 22.8. [M+Na]+ calculated for C34H41N5O13: 750.259, from MALDI-ToF spectrometry: 750.697.

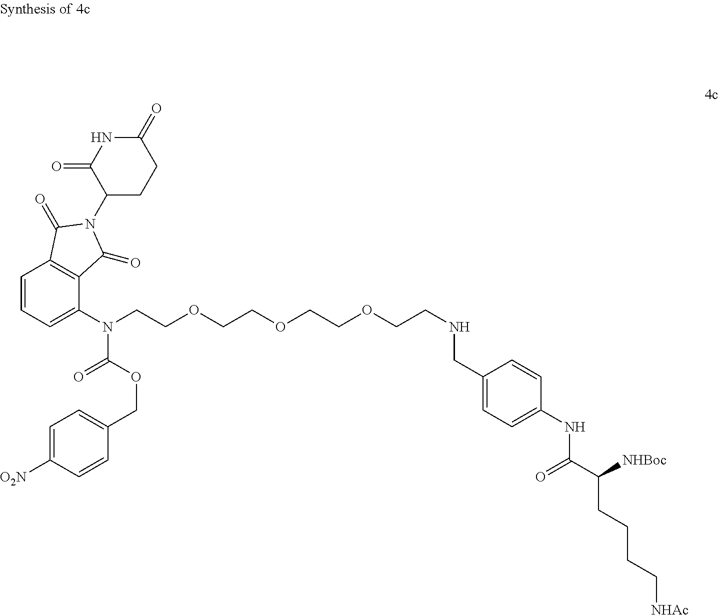 [SMILES: CC(=O)NCCCC[C@H](NC(=O)OC(C)(C)C)C(=O)Nc1ccc(CNCCOCCOCCOCCN(C(=O)OCc2ccc([N+](=O)[O-])cc2)c2cccc3c2C(=O)N(C2CCC(=O)NC2=O)C3=O)cc1]

10 mg (0.016 mmol) of 4b was dissolved in anhydrous THF (500 μL) and added to vial with excess anhydrous sodium sulfate (100 eq.) under inert atmosphere and the reaction mixture was stirred for 10 minutes. To this, 8 mg (0.019 mmol) 2c was dissolved anhydrous THF (500 μL) and added dropwise, following which the reaction mixture was stirred for 12 h. Sodium triacetoxyborohydride (0.019 mmol) was then added to the reaction mixture and stirred overnight. The reaction mixture was concentrated in vacuo, and the crude product was purified by reverse phase C-18 gel chromatography using water and methanol as eluents to afford 4c in 53% yield. 1H NMR (400 MHz, CH3OD) δ 9.90 (s, 1H), 7.90 (d, J=8.5 Hz, 2H), 7.84 (d, J=8.5 Hz, 2H), 7.60 (d, J=7.1 Hz, 1H), 7.58 (d, J=7.1 Hz, 1H), 7.37 (m, 1H), 7.31-7.29 (m, 1H), 7.29-7.26 (m, 2H), 7.23-7.21 (m, 2H), 7.13 (d, J=8.7 Hz, 1H), 7.08 (d, J=8.7 Hz, 1H), 5.10-5.06 (m, 1H), 4.61 (s, 2H), 4.18 (broad, 1H), 3.77-3.74 (m, 2H), 3.70-3.68 (m, 8H), 3.67-3.66 (m, 2H), 3.63-3.59 (m, 2H), 3.55-3.53 (m, 2H), 3.20-3.17 (m, 2H), 2.80-2.74 (m, 2H), 2.18-2.12 (m, 4H), 1.92 (s, 3H), 1.81-1.77 (m, 1H), 1.73-1.70 (m, 1H), 1.56-1.55 (m, 1H), 1.53 (d, J=6.9 Hz, 1H), 1.47 (s, 9H), 1.32-1.31 (m, 2H). 13C NMR (100 MHz, CH3OD) δ 173.2, 170.3, 169.4, 168.8, 167.8, 148.9, 146.8, 137.7, 135.8, 132.5, 128.8, 121.0, 120.1, 116.8, 110.7, 109.9, 70.2, 70.1, 70.0, 69.8, 69.2, 66.4, 59.1, 48.31, 48.1, 41.8, 41.1, 39.2, 30.7, 29.4, 22.8, 22.4. [M+Na]+ calculated for C49H62N8O15: 1025.419, from MALDI-ToF spectrometry: 1025.569.

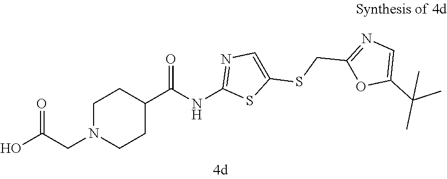 [SMILES: CC(C)(C)c1cnc(CSc2cnc(NC(=O)C3CCN(CC(=O)O)CC3)s2)o1]

To activated powdered 4 Å molecular sieves (50 eq.) in anhydrous dichloromethane (500 μL), was added cesium hydroxide monohydrate (8 mg, 0.027 mmol), and the white suspension was vigorously stirred for 10 min. Subsequently, 10 mg SNS-032 (4e) (0.027 mmol) was added and stirred for an additional 30 min, 6 mg tert-butyl bromoacetate (0.031 mmol) was added into the white suspension. The reaction was stirred for 12 h concentrated to a nominal volume by blowing air, the residue was extracted with brine (×3). The combined extracts were dried over anhydrous Na2SO4, and the crude product purified by silica gel chromatography using hexanes and ethyl acetate as eluents to afford the tert-butyl protected 4d in 96% yield. 1H NMR (400 MHz, CDCl3) δ 6.97 (s, 1H), 6.59 (s, 1H), 4.66 (s, 2H), 3.95 (s, 2H), 3.07-3.03 (m, 1H), 2.59-2.52 (m, 1H), 2.46 (m, 1H), 1.56-1.50 (m, 2H), 1.45 (s, 9H), 1.23 (s, 9H), 0.85 (m, 2H). 13C NMR (100 MHz, CDCl3) δ 169.1, 165.5, 161.8, 158.6, 132.1, 120.1, 114.7, 83.3, 77.4, 77.0, 76.7, 60.4, 52.2, 49.4, 33.7, 31.4, 29.7, 28.5, 28.1, 27.9, 27.6. [M+Na]+ calculated for C19H26N4O4S2: 439.147, from MALDI-ToF spectrometry: 439.257. The tert-butyl group was then deprotected using TFA in DCM (1:1) and stirred overnight. The resulting mixture was co-evaporated with methanol thrice and further concentrated in vacuo to yield 4d and used subsequently without any further purification.

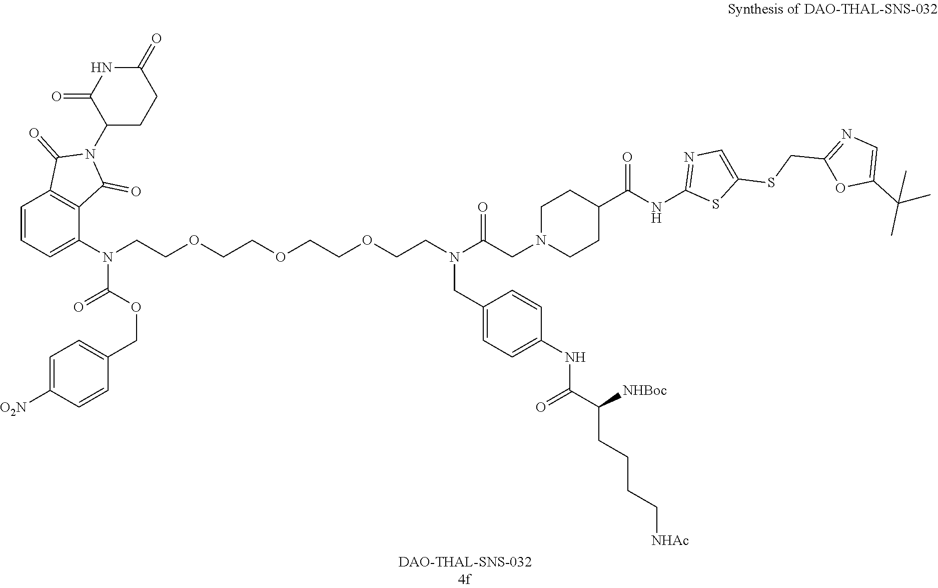 [SMILES: CC(=O)NCCCC[C@H](NC(=O)OC(C)(C)C)C(=O)Nc1ccc(CN(CCOCCOCCOCCN(C(=O)OCc2ccc([N+](=O)[O-])cc2)c2cccc3c2C(=O)N(C2CCC(=O)NC2=O)C3=O)C(=O)CN2CCC(C(=O)Nc3ncc(SCc4ncc(C(C)(C)C)o4)s3)CC2)cc1]

2 mg (0.006 mmol) 4d, 3 mg (0.008 mmol) HATU and 1 μL (0.006 mmol) DIPEA was dissolved in anhydrous dimethylformamide (250 μL) and added to a vial under inert atmosphere and the reaction mixture was stirred for 30 minutes. To this, 4 mg (0.004 mmol) 4c and 1 μL (0.006 mmol) DIPEA dissolved in anhydrous dimethylformamide (250 μL) was added dropwise to the reaction mixture and left to stir for 12 h. The reaction mixture was concentrated in vacuo, and the crude product purified by reverse phase C-18 gel chromatography using water and methanol as eluents to afford 4f in 60% yield. 1H NMR (400 MHz, (CD3)2SO) δ 10.48 (s, 1H), 9.88 (s, 1H), 7.87 (d, J=8.8 Hz, 2H), 7.83 (d, J=8.8 Hz, 2H), 7.38 (s, 1H), 7.23-7.19 (m, 2H), 7.15-7.13 (m, 2H), 7.11-7.10 (m, 1H), 7.09-7.02 (m, 1H), 6.94 (m, 1H), 6.72 (s, 1H), 4.83-4.76 (m, 1H), 4.48-4.45 (m, 1H), 4.05 (s, 2H), 3.61 (s, 2H), 3.51 (s, 2H), 2.87-2.84 (m, 1H), 2.80 (s, 1H), 2.47-2.44 (m, 2H), 1.77 (s, 3H), 1.73 (d, J=6.5 Hz, 2H), 1.65-1.58 (m, 2H), 1.38 (s, 9H), 1.28 (s, 1H), 1.28 (s, 1H), 1.24 (s, 2H), 1.18 (s, 9H), 0.96 (d, J=6.5 Hz, 1H), 0.86-0.84 (m, 1H). [M+H]+ calculated for C68H86N12O18S2: 1423.568, [M+H]+ from MALDI-ToF spectrometry: 1423.123.

### Material and Methods for Biological Evaluation

Cell culture: All cell lines used in this example were purchased from the American Type Culture Collection (ATCC). All cell lines were cultured in 75 cm² cell culture flasks with Dulbecco's Modified Eagle Medium/Nutrient Mixture F-12 (DMEM/F12, Gibco™ Catalog number: 11320033) in a humidified incubator with 5% CO₂ at 37° C. DMEM/F12 media was supplemented with 10% fetal bovine serum (FBS, regular, Corning® Catalog number: 35-010-CV), 1% Pen-Strep (10,000 U/mL, Gibco™ Catalog number: 15140122). All cell lines were examined as mycoplasma free.

Western Blotting: Cell lines were cultured in 12 well cell culture plates with 300,000 cells per well. Hypoxia was induced through treatment with CoCl₂ (200 PM, Sigma-Aldrich, CAS: 7791-13-1) for 24 h. Cathepsin L activity was blocked through treatment with Z-Phe-Tyr-CHO (100 μM, MedChem Express, CAS: 167498-29-5) for 24 h. After incubation, the culture medium was removed, and cells were treated with fresh media containing PROTAC or pro-PROTAC at different concentrations for 24 h along with or without CoCl₂ or Z-Phe-Tyr-CHO, as defined by the experiment. After treatment, cells were lysed with RIPA buffer (Thermo Scientific™, #89900) containing Protease/Phosphatase Inhibitor Cocktail (1×) (Cell Signaling, #5872). Gels were inserted into the gel apparatus and buffer was loaded till the fill line. Protein Ladder (PageRuler™ Plus Prestained, #26619) and samples mixed with blue loading buffer (Cell Signaling, #7722) were then loaded into gels that were run at 130 V for 1 h till the dye reaches the bottom of the gel. The gels were soaked in ethanol (20% v/v in DI water, 5 min) and then transferred using the gel transfer device (Invitrogen™ iBlot™ 2, or Invitrogen™ iBlot™ 3) using a method ideal for their molecular weight. The membranes were then blocked using blocking buffer, i.e., 5% (w/v) non-fat dry milk (Cell Signaling, #9999) in TBS Tween 20 Buffer (Thermo Scientific™, #28360) for 1 h before primary immunodetection using complimentary antibody (diluted to 1:2000 in blocking buffer) at 4° C. overnight with constant stirring. The membranes were then washed with TBS Tween 20 Buffer (×2) before secondary immunodetection using a secondary antibody (diluted to 1:10000 in blocking buffer). The membranes were then washed with TBS Tween 20 Buffer (×2) and incubated with an imaging reagent (1:1 Clarity Western Peroxide Reagent and Western Luminol/Enhancer Reagent, Bio-Rad, #1705061) for 10 min. The chemiluminescence images were taken using an imaging system (ChemiDoc™, Bio-Rad). Chemiluminescence intensity was quantified using Image Lab software. Antibodies in the study include α-Tubulin (DM1A) Mouse mAb (Cell Signaling, #3873), BRD4 (E2A7×) Rabbit mAb (Cell Signaling, #13440), c-Myc (D84C12) Rabbit mAb (Cell Signaling, #5605), ZFP-91 Rabbit pAb (Bethyl Laboratories, #A303-245A), Vinculin (E1E9V) XP® Rabbit mAb (Cell Signaling, #13901), Cathepsin L Mouse mAb (Thermo Scientific™, #BMSio32), HIF-1α Rabbit pAb (Thermo Scientific™, #PA1-16601), Goat Anti-Rabbit IgG H&L (HRP) (Abeam, #ab97051), Goat anti-Mouse IgG (H+L), Superclonal™ Recombinant Secondary Antibody, HRP (Thermo Scientific™, #A28177).

Cell Viability Assay: Cells were seeded on white flat-bottom 96-well tissue culture plates at a density of 5000 cells/well and rested for 24 h, then incubated for 24 h with CoCl₂ (200 μM, Sigma-Aldrich, CAS: 7791-13-1) to induce hypoxia or with Z-Phe-Tyr-CHO (100 μM, MedChem Express, CAS: 167498-29-5) to inhibit cathepsin L activity at 37° C. in 5% CO₂. (See Muñoz-Sánchez, J.; Chánez-Cárdenas, M. E. The Use of Cobalt Chloride as a Chemical Hypoxia Model. J. Appl. Toxicol. 2019, 39, 556-570; and Ueki, N.; Lee, S.; Sampson, N. S.; Hayman, M. J. Selective Cancer Targeting with Prodrugs Activated by Histone Deacetylas-es and a Tumour-Associated Protease. Nat. Commun. 2013, 4, 2735). After incubation, cells were treated with PROTAC/pro-PROTAC at different concentrations for 72 h. After treatments, the cells were treated with the reagent (CellTiter-Glo® 2.0, Promega, #G9243), covered and stirred using a plate shaker for 3 min and allowed to rest for 10 min at room temperature. The luminescent signal was measured using a SpectraMax iD5 multimode microplate reader. All studies were performed in triplicate.

Cell Apoptosis Assay: Cells were seeded on white flat-bottom 96-well tissue culture plates at a density of 5000 cells/well and rested for 24 h, then incubated for 24 h with CoCl₂ to induce hypoxia or with Z-Phe-Tyr-CHO to inhibit cathepsin L activity at 37° C. in 5% CO₂. After incubation, cells were treated with PROTAC/pro-PROTAC at different concentrations for 72 h. After treatments, the cells were treated with the reagent (Caspase-Glo® 3/7 Assay, Promega, #G8091), covered and stirred using a plate shaker for 1 min and allowed to rest for 30 min at room temperature in the dark. The luminescent signal was measured using a SpectraMax iD5 multimode microplate reader. All studies were performed in four replicates.

Annexin V and PI Staining for Apoptosis by Flow Cytometry: All cell lines were seeded in 96-well clear flat-bottom plates at a density of 10000 cells/well, rested for 24 h and then incubated for 24 h with CoCl₂ to induce hypoxia or with Z-Phe-Tyr-CHO to inhibit cathepsin L activity at 37° C. in 5% CO₂. After incubation, cells were treated with PROTAC/pro-PROTAC at different concentrations for 72 h. After incubation, the cells were washed twice with cold PBS and then resuspended in 1× Binding Buffer (50 μL/well, BD Biosciences, #556454, diluted to 1× prior to use) in V-bottom clear 96 well plate. Annexin V-FITC (2 μL/well, BD Biosciences, #556420, #556419) and Propidium Iodide (PI) (1 μL/well, BD Biosciences, #556463) were added, gently mixed and incubated for 15 minutes at room temperature in the dark. Cells were then centrifuged at 500 ref for 5 min, and the incubation buffer was replaced with fresh 1× Binding Buffer (50 μL). The samples were analyzed by flow cytometry within one hour on a BD LSRFortessa flow cytometer and the data collected was analyzed on FlowJo™ Software. All studies were performed in four replicates.

### REFERENCES FOR EXAMPLES

The references cited below are hereby incorporated by reference to disclose and describe the methods or materials in connection with which the publications are cited or to provide background for the present disclosure. Any incorporation by reference of documents below is limited such that no subject matter is incorporated by reference that is contrary to the explicit disclosure herein. In the event of inconsistent usages between this document and those documents so incorporated by reference below, the use in the incorporated references should be considered supplementary to that of this document; for irreconcilable inconsistencies, the usage in this document controls.
(1) Kastl, J. M.; Davies, G.; Godsman, E.; Holdgate, G. A. Small-Molecule Degraders beyond PROTACs—Challenges and Opportunities. SLAS Discovery 2021, 26 (4), 524-533.
(2) Nalawansha, D. A.; Crews, C. M. PROTACs: An Emerging Therapeutic Modality in Precision Medicine. Cell Chem. Biol. 2020, 27 (8), 998-1014.
(3) Sun, X.; Gao, H.; Yang, Y.; He, M.; Wu, Y.; Song, Y.; Tong, Y.; Rao, Y. Protacs: Great Opportunities for Academia and Industry. Signal Transduct. Target Ther. 2019, 4 (1), 64.
(4) Guenette, R. G.; Yang, S. W.; Min, J.; Pei, B.; Potts, P. R. Target and Tissue Selectivity of PROTAC Degraders. Chem. Soc. Rev. 2022, 51 (14), 5740-5756.
(5) Chen, C.; Yang, Y.; Wang, Z.; Li, H.; Dong, C.; Zhang, X. Recent Advances in Pro-PROTAC Development to Address On-Target Off-Tumor Toxicity. J. Med. Chem. 2023, 66 (13), 8428-8440.
(6) Pfaff, P.; Samarasinghe, K. T. G.; Crews, C. M.; Carreira, E. M. Reversible Spatiotemporal Control of Induced Protein Degradation by Bistable PhotoPROTACs. ACS Cent. Sci. 2019, 5 (10), 1682-1690.
(7) Liu, H.; Ren, C.; Sun, R.; Wang, H.; Zhan, Y.; Yang, X.; Jiang, B.; Chen, H. Reactive Oxygen Species-Responsive Pre-PROTAC for Tumor-Specific Protein Degradation. Chem. Commun. 2022, 58, 10072-10075.
(8) Yang, C.; Yang, Y.; Li, Y.; Ni, Q.; Li, J. Radiotherapy-Triggered Proteolysis Targeting Chimera Prodrug Activation in Tumors. J. Am. Chem. Soc. 2023, 145 (1), 385-391.
(9) Shi, S.; Du, Y.; Zou, Y.; Niu, J.; Cai, Z.; Wang, X.; Qiu, F.; Ding, Y.; Yang, G.; Wu, Y.; Xu, Y.; Zhu, Q. Rational Design for Nitroreductase (NTR)-Responsive Proteolysis Targeting Chimeras (PROTACs) Selectively Targeting Tumor Tissues. J. Med. Chem. 2022, 65 (6), 5057-5071.
(10) Xue, G.; Wang, K.; Zhou, D.; Zhong, H.; Pan, Z. Light-Induced Protein Degradation with Photocaged PROTACs. J. Am. Chem. Soc. 2019, 141 (46), 18370-18374.
(11) Gao, J.; Wu, P.; Fernandez, A.; Zhuang, J.; Thayumanavan, S.; Cellular AND Gates: Synergistic Recognition to Boost Selective Uptake of Polymeric Nanoassemblies. Angew. Chem. Int. Ed. 2020, 59 (26), 10456-10460.
(12) Godet, I.; Doctorman, S.; Wu, F.; Gilkes, D. M. Detection of Hypoxia in Cancer Models: Significance, Challenges, and Advances. Cells 2022, 11 (4), 686.
(13) Ueki, N.; Lee, S.; Sampson, N. S.; Hayman, M. J. Selective Cancer Targeting with Prodrugs Activated by Histone Deacetylases and a Tumour-Associated Protease. Nat. Commun. 2013, 4, 2735.
(14) Linders, D. G. J.; Bijlstra, O. D.; Fallert, L. C.; Hilling, D. E.; Walker, E.; Straight, B.; March, T. L.; Valentijn, A. R. P. M.; Pool, M.; Burggraaf, J.; Basilion, J. P.; Vahrmeijer, A. L.; Kuppen, P. J. K. Cysteine Cathepsins in Breast Cancer: Promising Targets for Fluorescence-Guided Surgery. Mol. Imaging Biol. 2023, 25, 58-73.
(15) Winter, G. E.; Buckley, D. L.; Paulk, J.; Roberts, J. M.; Souza, A.; Dhe-Paganon, S.; Bradner, J. E. Phthalimide Conjugation as a Strategy for in vivo Target Protein Degradation. Science 2015, 348, 1376-1381.
(16) Zagidullin, A.; Milyukov, V.; Rizvanov, A.; Bulatov, E. Novel approaches for the rational design of PROTAC linkers. Explor. Targeted Antitumor Ther. 2020, 1, 381-90.
(17) O'Connor, L. J.; Cazares-Körner, C.; Saha, J.; Evans, C. N. G.; Stratford, M. R. L.; Hammond, E. M.; Conway, S. J. Design, Synthesis and Evaluation of Molecularly Targeted Hypoxia-Activated Prodrugs. Nat. Protoc. 2016, 11 (4), 781-794.
(18) Beasley, S.; Vandewalle, A.; Singha, M.; Nguyen, K.; England, W.; Tarapore, E.; Dai, N.; Corrêa, I. R.; Atwood, S. X.; Spitale, R. C. Exploiting Endogenous Enzymes for Cancer-Cell Selective Metabolic Labeling of RNA in Vivo. J. Am. Chem. Soc. 2022, 144 (16), 7085-7088.
(19) Muñoz-Sánchez, J.; Chánez-Cárdenas, M. E. The Use of Cobalt Chloride as a Chemical Hypoxia Model. J. Appl. Toxicol. 2019, 39, 556-570.
(20) Nguyen, T. M.; Sreekanth, V.; Deb, A.; Kokkonda, P.; Tiwari, P. K.; Donovan, K. A.; Shoba, V.; Chaudhary, S. K.; Mercer, J. A. M.; Lai, S.; Sadagopan, A.; Jan, M.; Fischer, E. S.; Liu, D. R.; Ebert, B. L.; Choudhary, A. Proteolysis-Targeting Chimeras with Reduced off-Targets. Nat. Chem. 2024, 16, 218-228.
(21) Da Motta, L. L.; Ledaki, I.; Purshouse, K.; Haider, S.; De Bastiani, M. A.; Baban, D.; Morotti, M.; Steers, G.; Wigfield, S.; Bridges, E.; Li, J. L.; Knapp, S.; Ebner, D.; Klamt, F.; Harris, A. L.; McIntyre, A. The BET Inhibitor JQ1 Selectively Impairs Tumour Response to Hypoxia and Downregulates CA9 and Angiogenesis in Triple Negative Breast Cancer. Oncogene 2017, 36 (1), 122-132
(22) Olson, C. M.; Jiang, B.; Erb, M. A.; Liang, Y.; Doctor, Z. M.; Zhang, Z.; Zhang, T.; Kwiatkowski, N.; Boukhali, M.; Green, J. L.; Haas, W.; Nomanbhoy, T.; Fischer, E. S.; Young, R. A.; Bradner, J. E.; Winter, G. E.; Gray, N. S. Pharmacological Perturbation of CDK9 Using Selective CDK9 Inhibition or Degradation. Nat. Chem. Biol. 2018, 14 (2), 163-170.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

What is claimed is:

1. A proteolysis targeting chimera (PROTAC) of Formula I: 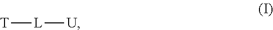
or a pharmaceutically acceptable salt or derivative thereof;
wherein:
T comprises a target binding moiety capable of binding to a target protein;
L comprises a linker moiety;
U comprises a ubiquitin ligase-recruiting moiety capable of binding a ubiquitin ligase;
wherein any of T, L, or U further comprises a first stimulus-reactive moiety Si, wherein S1 is reactive to a first stimulus; and
wherein any of T, L, or U further comprises a second stimulus-reactive moiety S2, wherein S2 is reactive to a second stimulus, wherein the second stimulus is different than the first stimulus.

2. The PROTAC of claim 1, wherein upon exposure to only the first stimulus, only the second stimulus, or to neither of the first stimulus or second stimulus, the PROTAC of Formula I does not substantially recruit the target protein for ubiquitination and degradation by a proteasome.

3. The PROTAC of claim 1, wherein upon exposure to both the first stimulus and the second stimulus, the PROTAC of Formula I forms an active compound capable of recruiting the target protein for ubiquitination and degradation by a proteasome.

4. The PROTAC of claim 1, wherein the first stimulus, the second stimulus, or both independently comprise one or more stimuli present within a cell.

5. The PROTAC of claim 1, wherein the first stimulus, the second stimulus, or both independently comprise one or more stimuli delivered exogenously to a cell.

6. The PROTAC of claim 1, wherein the first stimulus, the second stimulus, or both independently comprise one or more stimuli associated with a disease.

7. The PROTAC of claim 1, wherein the first stimulus, the second stimulus, or both independently comprise hypoxia, reactive oxygen species, glutathione, one or more enzymes, light, or radiation.

8. The PROTAC of claim 1, wherein L comprises the first stimulus-reactive moiety S1, the second stimulus-reactive moiety S2, or both.

9. The PROTAC of claim 1, wherein S¹ or S² is

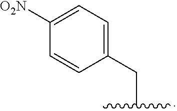 [SMILES: *Cc1ccc([N+](=O)[O-])cc1]

10. The PROTAC of claim 1, wherein S¹ or S²

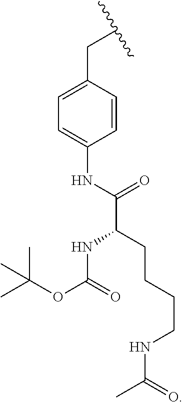 [SMILES: *Cc1ccc(NC(=O)[C@H](CCCCNC(C)=O)NC(=O)OC(C)(C)C)cc1]

11. The PROTAC of claim 1, wherein the linker moiety is selected from L1 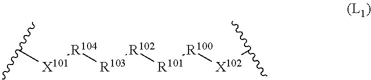
wherein:
X¹⁰¹ and X¹⁰² are independently at each occurrence selected from a bond, 6- to 10-membered monocyclic or bicyclic aryl, 5- to 10-membered monocyclic or bicyclic heteroaryl, C₃-C₇ cycloalkyl, 3- to 8-membered monocyclic or bicyclic heterocycle, C₁-C₂₀ alkyl, C₂-C₂₀ alkenyl, C₂-C₂₀ alkynyl, NR¹⁰⁵—, O, C(O), and S;
R¹⁰⁰, R¹⁰¹, R¹⁰², R¹⁰³, and R¹⁰⁴ are independently at each occurrence selected from the group consisting of a bond, —C(O)—, —C(O)O—, —OC(O)—, —SO₂—, —S(O)—, C(S)—, —C(O)NR¹⁰⁵—, —NR¹⁰⁵C(O)—, —O—, —S—, —NR¹⁰⁵—, —P(O)(OR¹⁰⁵))—, C₁-C₂₀ alkyl, C₂-C₂₀ alkenyl, C₂-C₂₀ alkynyl, 6- to 10-membered monocyclic or bicyclic aryl, 3- to 8-membered monocyclic or bicyclic heterocycle, C₃-C₇ cycloalkyl, 5- to 10-membered monocyclic or bicyclic heteroaryl, lactic acid, or glycolic acid, each of which may be optionally substituted with one or more (for example, 1, 2, 3, or 4) substituents independently selected from R¹⁴⁰;
R¹⁰⁵ independently selected at each occurrence from R¹²⁰, R¹¹⁰C(O)—, R¹¹⁰C(N)—, R¹¹⁰S(O)—, and R¹¹⁰S(O)₂—, each of which may be optionally substituted with one or more R¹⁴⁰ groups as allowed by valency;
R¹¹⁰ is independently selected at each occurrence from hydrogen, halo, C₁-C₆alkyl, C₁-C₆haloalkyl, C₂-C₆alkenyl, C₂-C₆alkynyl, (C₃-C₇ cycloalkyl)-(C₀-C₆ alkyl)-, (4- to 6-membered heterocycle)-(C₀-C₆ alkyl)-, (5- to 10-membered monocyclic or bicyclic aryl)-(C₀-C₆ alkyl)-, (5- to 10-membered monocyclic or bicyclic heteroaryl)-(C₀-C₆ alkyl)-, —OR¹²⁰, —SR¹²⁰, and —NR¹²⁰R¹³⁰, each of which may be optionally substituted with one or more R¹⁴⁰ groups as allowed by valency;
R¹²⁰ and R¹³⁰ are independently selected at each occurrence from hydrogen, C₁-C₆alkyl, C₁-C₆haloalkyl, C₂-C₆alkenyl, C₂-C₆alkynyl, (C₃-C₇ cycloalkyl)-(C₀-C₆ alkyl)-, (4- to 6-membered heterocycle)-(C₀-C₆ alkyl)-, (5- to 10-membered monocyclic or bicyclic aryl)-(C₀-C₆ alkyl)-, (5- to 10-membered monocyclic or bicyclic heteroaryl)-(C₀-C₆ alkyl)-, each of which may be optionally substituted by one or more R¹⁴⁰ groups as allowed by valency; and
R¹⁴⁰ is independently at each occurrence selected from the group consisting of hydrogen, halo, nitro, cyano, azido, oxo, C₁-C₆ alkyl, C₁-C₆ haloalkyl, C₂-C₆ alkenyl, C₂-C₆ alkynyl, (C₃-C₇ cycloalkyl)(C₀-C₆ alkyl)-, (3- to 8-membered monocyclic or bicyclic heterocycle)-(C₀-C₆ alkyl)-, (6- to 10-membered monocyclic or bicyclic aryl)-(C₀-C₆ alkyl)-, (5- to 10-membered monocyclic or bicyclic heteroaryl)-(C₀-C₆ alkyl)-, R¹⁴¹O—, R¹⁴¹S—, R¹⁴¹R¹⁴²N—, R¹⁴³C(O)—, R¹⁴³C(O)—O—, R¹⁴²C(O)—NR¹⁴²—, R¹⁴³S(O)₂—, R¹⁴³S(O)₂—O—, and R¹⁴³S(O)₂—NR¹⁴²—, wherein R¹⁴³ is independently selected at each occurrence from R¹⁴¹, halo, R¹⁴¹O—, and R¹⁴¹R¹⁴²N—, and wherein R¹⁴¹ and R¹⁴² are independently selected at each occurrence from hydrogen, C₁-C₆ alkyl, C₁-C₆ haloalkyl, C₂-C₆ alkenyl, C₂-C₆ alkynyl, (C₃-C₇ cycloalkyl)(C₀-C₆ alkyl)-, (3- to 8-membered monocyclic or bicyclic heterocycle)-(C₀-C₆ alkyl)-, (6- to 10-membered monocyclic or bicyclic aryl)-(C₀-C₆ alkyl)-, and (5- to 10-membered monocyclic or bicyclic heteroaryl)-(C₀-C₆ alkyl)-;
wherein each of X¹⁰¹, X¹⁰², R¹⁰⁰, R¹⁰¹, R¹⁰², R¹⁰³, and R¹⁰⁴ may be optionally substituted by S¹ or S¹.

12. The PROTAC of claim 1, wherein L is selected from

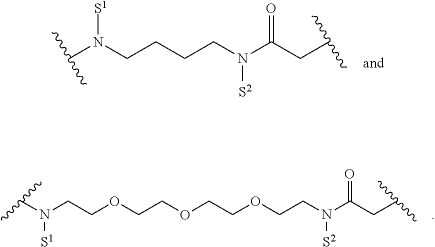

13. The PROTAC of claim 1, wherein the target binding moiety comprises or is derived from a ligand of the target protein.

14. The PROTAC of claim 1, wherein the target binding moiety comprises or is derived from a ligand for BRD4 or CDK9.

15. The PROTAC of claim 1, wherein the target binding moiety is selected from

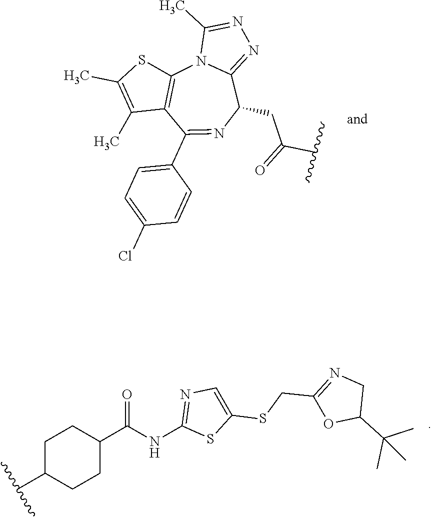 [SMILES: *C(=O)C[C@@H]1N=C(c2ccc(Cl)cc2)c2c(sc(C)c2C)-n2c(C)nnc21 ; *C1CCC(C(=O)Nc2ncc(SCC3=NCC(C(C)(C)C)O3)s2)CC1]

16. The PROTAC of claim 1, wherein the ubiquitin ligase-recruiting moiety is capable of binding to cereblon.

17. The PROTAC of claim 1, wherein the ubiquitin ligase-recruiting moiety is selected from

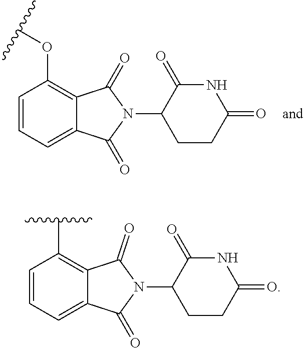 [SMILES: *Oc1cccc2c1C(=O)N(C1CCC(=O)NC1=O)C2=O ; *c1cccc2c1C(=O)N(C1CCC(=O)NC1=O)C2=O]

18. A pharmaceutical composition comprising a PROTAC of claim 1, or a pharmaceutically acceptable salt or derivative thereof, and a pharmaceutically acceptable carrier or excipient.

19. A method of treating a medical disorder that is mediated by a target protein in a subject in need thereof, the method comprising administering a therapeutically effective amount of a PROTAC of claim 1, or a pharmaceutically acceptable salt or derivative thereof, wherein the target protein is degraded or downregulated by the PROTAC.

20. The method of claim 17, wherein the medical disorder is a cancer, a neurodegenerative disorder, or an autoimmune disorder.
